SEQUENCE LISTING (20 sequences)

SEQ ID NO: 1 - DNA, 18 bp, Saccharomyces cerevisiae
ggtggtaggcacggatcc

SEQ ID NO: 2 - DNA, 18 bp, Saccharomyces cerevisiae
gaaggcgggtcgaccggt

SEQ ID NO: 3 - DNA, 27 bp, Saccharomyces cerevisiae
gaaggcgggtcgaccatgtcggggggg

SEQ ID NO: 4 - DNA, 15 bp, Saccharomyces cerevisiae
attgggtcgaccggt

SEQ ID NO: 5 - DNA, 15 bp, Saccharomyces cerevisiae
atagggtcgaccggt

SEQ ID NO: 6 - DNA, 39 bp, Saccharomyces cerevisiae
atagggtcgaccttaatgcagagatctggcatcatggtt

SEQ ID NO: 7 - DNA, 42 bp, Saccharomyces cerevisiae
aacggcgggtcgaccttaatgcagagatctggcatcatggtt

SEQ ID NO: 8 - DNA, 15 bp, Saccharomyces cerevisiae
gacgggtcgaccggt

SEQ ID NO: 9 - DNA, 14 bp, Saccharomyces cerevisiae
ggatccctggcgtc

SEQ ID NO: 10 - DNA, 20 bp, Saccharomyces cerevisiae
ggatccctgggtctgggatg

SEQ ID NO: 11 - DNA, 17 bp, Saccharomyces cerevisiae
ggatccctggggatatg

SEQ ID NO: 12 - DNA, 17 bp, Saccharomyces cerevisiae
ggatccctggggattcc

SEQ ID NO: 13 - DNA, 57 bp, Artificial Sequence
  primer
ccccctctttcattgtggtcaccgtttttgaacacaaccagctgaagcttcgtacgc

SEQ ID NO: 14 - DNA, 60 bp, Artificial Sequence
  primer
cacaaaaatccagtccataactaacacaatcattactagcataggccactaggtgatctg

SEQ ID NO: 15 - DNA, 29 bp, Artificial Sequence
  primer
gccaagcttatgcagattttcgtcaagac

SEQ ID NO: 16 - DNA, 27 bp, Artificial Sequence
  primer
gccagatctccagcgtaatctggaaca

SEQ ID NO: 17 - DNA, 28 bp, Artificial Sequence
  primer
gccagatctgccagcgtaatctggaaca

SEQ ID NO: 18 - DNA, 29 bp, Artificial Sequence
  primer
gccagatctggccagcgtaatctggaaca

SEQ ID NO: 19 - DNA, 30 bp, Artificial Sequence
  primer
gccaggcctcatgagtaaaggagaagaact

SEQ ID NO: 20 - DNA, 31 bp, Artificial Sequence
  primer
gccgggccctatttgtatagttcatccatgc

(19) United States
(12) Patent Application Publication
(10) Pub. No.: US 2004/0170970 A1
(43) Pub. Date: Sep. 2, 2004

(54) Split- ubiquitin based reporter systems and methods of their use

(76) Inventors: Alexander Varshavsky, Flintridge, CA (US); Sandra Wittke, Koeln (DE); Nils Johnsson, Koeln (DE); Norbert Lehming, Koeln (DE)

Correspondence Address: ROPES & GRAY LLP, ONE INTERNATIONAL PLACE, BOSTON, MA, 02110-2624, US

(21) Appl. No.: 09/923,917

(22) Filed: Aug. 6, 2001

Related U.S. Application Data
(60) Provisional application No. 60/223,411, filed on Aug. 4, 2000.

(51) Int. Cl. C12Q 1/68; C07H 21/04; C12P 21/04; C12N 9/64

(52) U.S. Cl. 435/6; 435/69.7; 435/226; 435/320.1; 435/325; 536/23.2

(57) ABSTRACT

Methods and reagents for the detection and selection of two interacting-polypeptides, especially integral membrane proteins and transcription factors, by monitoring the reassembly of ubiquitin amino-terminal and carboxy-terminal chimeric polypeptide fragments are disclosed. Negative selection against an N-end rule-labilized marker released following ubiquitin reassembly allows direct selection of the interacting polypeptide pair. Methods to identify agonists and antagonists for certain protein-protein interactions; methods and reagents/kits for identifying proteins that binds a target protein are also provided. The dynamic and adaptable nature of the assay allows adaptation to a number of applications—such as probing the molecular environment of cellular membrane proteins in vivo.

### REFERENCE TO RELATED APPLICATIONS

This application claims priority to Provisional application 60/223,41 1, filed on Aug. 4, 2000, the specification of which is incorporated by reference herein.

### 1. BACKGROUND OF THE INVENTION

Protein interactions facilitate most biological processes including signal transduction and homeostasis. The elucidation of particular interacting protein partners facilitating these biological processes has been advanced by the development of in vivo “two-hybrid” or “interaction trap” methods for detecting and selecting interacting protein partners (see Fields & Song (1989) Nature 340: 245-6; Gyuris et al. (1993) Cell 75: 791-803). These methods rely upon the reconstitution of a nuclear transcriptional activator via the interaction of two binding partner polypeptides—i.e. a first polypeptide fused to a DNA binding domain and a second polypeptide fused to a transcriptional activation domain. When the first and the second polypeptides interact, the interaction can be detected by the activation of a reporter gene containing binding sites for the DNA binding domain. For this method to work, both proteins need to be soluble and to be localized to the nucleus. Accordingly, the interaction of polypeptides which are normally localized to other compartments may not be detected because of the absence of other non-nuclear polypeptide components which facilitate the interaction or particular non-nuclear post-translational modifications which fail to occur in the nucleus or because the interacting proteins fail to fold properly when localized to the nuclear compartment. In particular, the nuclear two-hybrid assay is ill-suited to the detection of protein interactions occurring within or at the surface of cellular membranes. Membrane proteins, especially integral membrane proteins tend to be insoluble and form aggregates if not in their native membrane environment, partly due to the strong hydrophobicity of their membrane-associated domains/regions, such as the transmembrane region. Another category of protein that traditional yeast two-hybrid assay is ill-suited to study is transcription factors (both transcriptional activators and repressors) since these proteins, when serving as so-called “baits,” may interfere with the read-out of the assay—transcriptional activation of certain reporter genes.

The Split Ubiquitin Protein Sensor is described in U.S. Pat. Nos. 5,585,245 and 5,503,977. In brief, the “split ubiquitin” method (split-Ub) is a way of detecting protein-protein interactions that relies in part upon the fact that isolated amino-terminal- and carboxy-terminal-fragments of ubiquitin (e.g. comprising amino acids 1 to 37 and 38 to 76 respectively) are able to spontaneously associate to reconstitute a bimolecular ubiquitin polypeptide complex that is recognized by ubiquitin-specific proteases (UBPs), present in the cytosol and nucleus of all eukaryotic cells. UBPs recognize the reconstituted ubiquitin, but not its halves, and actively cleave off the polypeptide bond between amino acid residue 76 of the carboxyl fragment of ubiquitin and any linked polypeptide. If this linked polypeptide is a reporter which becomes activated upon release from the carboxy-terminal ubiquitin protein fragment, then the association of amino-terminal and carboxy-terminal ubiquitin fragments can be monitored by the activation of the reporter activity. This “re-association” of ubiquitin amino-terminal and carboxy-terminal fragments can be made dependent upon the association of two heterologous polypeptides by generating mutations in the ubiquitin fragments (e.g. by a conservative amino acid substitution of a neutral amino acid residue) so that they fail to “reassociate” without the aid of linked heterologous binding partners. The two heterologous polypeptides (i.e. a first polypeptide and a second polypeptide) are provided as fusions to the amino-terminal and the carboxy-terminal ubiquitin fragments. In addition, the carboxy-terminal ubiquitin fragment is fused at its C-terminus to a reporter gene. In certain cases, the resulting two fusions have the structures 1^{st} polypeptide-N-Ub*_{(1-Y)} and 2^{nd} polypeptide-C-Ub*_{(Z-76)}-reporter (wherein Y equals approximately 34-37, and Z equals approximately 35-38). In the absence of the interaction of the first and second polypeptides, the altered ubiquitin amino-terminal and carboxy-terminal fragments fail to associate. In contrast, association of the first and second polypeptides results in reassembly of the amino-terminal Ub* and carboxy-terminal Ub* fragments and cleavage of the carboxy-terminal Ub*-reporter bond, thereby releasing free reporter. If the reporter is active upon its release, but inactive while fused to the carboxy-terminal fragment of ubiquitin, its activity can be monitored in a screen for polypeptide binding partners (see U.S. Pat. Nos. 5,585,245 and 5,503,977).

The assay has been shown to detect interactions between cytosolic proteins, membrane proteins, and transient interactions that occur between transporter and substrate during protein translocation across the membrane of the endoplasmic reticulum in vivo. In addition, split-Ub can also be used to demonstrate interactions between transcription factors because, contrary to the two-hybrid system, it is not based on a transcriptional readout.

In a general review of the split-ub assay, potential use of the N-end rule was mentioned (Johnsson and Varshavsky, Chapter 19 in Adv. in Mol. Biol., Ed. Bartel, P. L. and Fields, S., Oxford University Press, 1997, Oxford). Also in that review is a suggestion of the assay in vitro and for detecting membrane protein interactions.

### 2. SUMMARY OF THE INVENTION

In general, the invention provides methods and reagents for the detection, selection or monitoring of interacting polypeptides, especially integral membrane proteins and transcription factors. In certain embodiments, the invention is used in cell-based assays for protein interaction. The assays include selection systems which allow selective growth of a eukaryotic cell, such as a yeast or a mammalian cell, when two test polypeptides interact with one another. These assays further provide methods for identifying compounds which act as agonist or antagonists of a particular polypeptide interaction. In addition, these assays provide methods and kits for identification of proteins that bind a target protein.

In one aspect, the invention provides a pair of fusion proteins consisting of a first fusion protein comprising segments P1, Cub-X, and RM, in an order wherein Cub-X is closer to the N-terminus of the first fusion protein than RM, and a second fusion protein comprising segments Nux and P2, wherein: P1 or P2 or both is a membrane-associated protein, and P2 may be the same or different from P1; Nux is the amino-terminal subdomain of a wild-type ubiquitin or a reduced-associating mutant ubiquitin amino-terminal subdomain; Cub is the carboxy-terminal subdomain of a wild-type ubiquitin; X is an amino acid other than methionine; RM is an reporter moiety, and, wherein the binding that occurs between P1 and P2 results in reassociation of Nux and Cub, thereby permitting ubiquitin-specific protease cleavage between Cub and X.

In a related aspect, the invention provides a pair of fusion proteins consisting of a first fusion protein comprising segments P1, Cub-X, and RM, in an order wherein Cub-X is closer to the N-terminus of the first fusion protein than RM, and a second fusion protein comprising segments Nux and P2, wherein: P1 or P2 or both is a transcription factor; Nux is the amino-terminal subdomain of a wild-type ubiquitin or a reduced-associating mutant ubiquitin amino-terminal subdomain; Cub is the carboxy-terminal subdomain of a wild-type ubiquitin; X is an amino acid other than methionine; RM is an reporter moiety, and, wherein the binding that occurs between P1 and P2 results in reassociation of Nux and Cub, thereby permitting ubiquitin-specific protease cleavage between Cub and X.

In a related aspect, the invention provides a pair of fusion proteins consisting of a first fusion protein comprising segments P1, Cub-X, and RM, in an order wherein Cub-X is closer to the N-terminus of the first fusion protein than RM, and a second fusion protein comprising segments Nux and P2, wherein: P1 or P2 or both is a membrane-associated protein, and P2 may be the same or different from P1; Nux is the amino-terminal subdomain of a wild-type ubiquitin or a reduced-associating mutant ubiquitin amino-terminal subdomain; Cub is the carboxy-terminal subdomain of a wild-type ubiquitin; X is an amino acid; RM is an enzymatically active reporter moiety, and, wherein the binding that occurs between P1 and P2 results in reassociation of Nux and Cub, thereby permitting ubiquitin-specific protease cleavage between Cub and X.

In a related aspect, the invention provides a pair of fusion proteins consisting of a first fusion protein comprising segments P1, Cub-X, and RM, in an order wherein Cub-X is closer to the N-terminus of the first fusion protein than RM, and a second fusion protein comprising segments Nux and P2, wherein: P1 or P2 or both is transcription factor; Nux is the amino-terminal subdomain of a wild-type ubiquitin or a reduced-associating mutant ubiquitin amino-terminal subdomain; Cub is the carboxy-terminal subdomain of a wild-type ubiquitin; X is an amino acid; RM is an enzymatically active reporter moiety, and, wherein the binding that occurs between P1 and P2 results in reassociation of Nux and Cub, thereby permitting ubiquitin-specific protease cleavage between Cub and X.

In one embodiment, X is Arginine. In a related embodyment, X is selected from the group consisting of Lysine, Histidine, Phenylalanine, Tryptophan, Tyrosine, Leucine, Aspartate, Glutamate, Cysteine, Asparagine, Glutamine and Isoleucine. In yet another related embodyment, X is Methionine, Glycine or Valine.

In one embodiment, the reporter moiety is a selectable marker. In a preferred embodiment, the selectable marker is selected from the group consisting of: URA3, HIS3, LYS2, HygTk, Tkneo, TkBSD, PACTk, HygCoda, Codaneo, CodaBSD, PACCoda, Tk, codA, HPRT, and GPT2. In a related preferred embodiment, the selectable marker is selected from the group consisting of: TRP1, CYH2, and CAN1.

In another embodiment, the reporter moiety is selected from the group consisting of: a transcription factor and a fluorescent marker.

In one embodiment, Nux contains at least one point mutation at amino acid 3 or amino acid 13 of a ubiquitin.

In another aspect, the invention provides one or more nucleic acids that encodes or that together encode a first fusion protein comprising segments P1, Cub-X, and RM, in an order wherein Cub-X is closer to the N-terminus of the first fusion protein than RM, and a second fusion protein comprising segments Nux and P2, wherein: P1 or P2 or both is a membrane-associated protein, and P2 may be the same or different from P1; Nux is the amino-terminal subdomain of a wild-type ubiquitin or a reduced-associating mutant ubiquitin amino-terminal subdomain; Cub is the carboxy-terminal subdomain of a wild-type ubiquitin; X is an amino acid other than methionine; RM is a reporter moiety, and, wherein the binding that occurs between P1 and P2 results in reassociation of Nux and Cub, thereby permitting ubiquitin-specific protease cleavage between Cub and X.

In a related aspect, the invention provides one or more nucleic acids that encodes or that together encode a first fusion protein comprising segments P1, Cub-X, and RM, in an order wherein Cub-X is closer to the N-terminus of the first fusion protein than RM, and a second fusion protein comprising segments Nux and P2, wherein: P1 or P2 or both is a membrane-associated protein, and P2 may be the same or different from P1; Nux is the amino-terminal subdomain of a wild-type ubiquitin or a reduced-associating mutant ubiquitin amino-terminal subdomain; Cub is the carboxy-terminal subdomain of a wild-type ubiquitin; X is an amino acid; RM is an enzymatically active reporter moiety, and, wherein the binding that occurs between P1 and P2 results in reassociation of Nux and Cub, thereby permitting ubiquitin-specific protease cleavage between Cub and X.

In a related aspect, the invention provides one or more nucleic acids that encodes or that together encode a first fusion protein comprising segments P1, Cub-X, and RM, in an order wherein Cub-X is closer to the N-terminus of the first fusion protein than RM, and a second fusion protein comprising segments Nux and P2, wherein: P1 or P2 or both is a transcription factor; Nux is the amino-terminal subdomain of a wild-type ubiquitin or a reduced-associating mutant ubiquitin amino-terminal subdomain; Cub is the carboxy-terminal subdomain of a wild-type ubiquitin; X is an amino acid other than methionine; RM is a reporter moiety, and, wherein the binding that occurs between P1 and P2 results in reassociation of Nux and Cub, thereby permitting ubiquitin-specific protease cleavage between Cub and X.

In a related aspect, the invention provides one or more nucleic acids that encodes or that together encode a first fusion protein comprising segments P1, Cub-X, and RM, in an order wherein Cub-X is closer to the N-terminus of the first fusion protein than RM, and a second fusion protein comprising segments Nux and P2, wherein: P1 or P2 or both is a transcription factor; Nux is the amino-terminal subdomain of a wild-type ubiquitin or a reduced-associating mutant ubiquitin amino-terminal subdomain; Cub is the carboxy-terminal subdomain of a wild-type ubiquitin; X is an amino acid; RM is an enzymatically active reporter moiety, and, wherein the binding that occurs between P1 and P2 results in reassociation of Nux and Cub, thereby permitting ubiquitin-specific protease cleavage between Cub and X.

In another aspect, the invention provides a method of determining whether two proteins, at least one of which is a membrane-associated protein, bind to each other comprising the steps of: translationally providing a first fusion protein comprising segments P1, Cub-X, and RM, in an order wherein Cub-X is closer to the N-terminus of the first fusion protein than RM, and a second fusion protein comprising segments Nux and P2, wherein P1 and P2 are proteins, at least one of which is membrane-associated, which proteins may be the same or different, Nux is the amino-terminal subdomain of a wild-type ubiquitin or a reduced-associating mutant ubiquitin amino-terminal subdomain, Cub is the carboxy-terminal subdomain of a wild-type ubiquitin, X is an amino acid other than methionine and RM is an active reporter moiety; and detecting the degree of cleavage by a ubiquitin-specific protease of the first fusion protein between Cub and X by detecting the degree of the activity of RM, wherein an increase of cleavage is indicative of P1/P2 binding.

In a related aspect, the invention provides a method of determining whether two proteins, at least one of which is a transcription factor, bind to each other comprising the steps of: translationally providing a first fusion protein comprising segments P1, Cub-X, and RM, in an order wherein Cub-X is closer to the N-terminus of the first fusion protein than RM, and a second fusion protein comprising segments Nux and P2, wherein P1 and P2 are proteins, at least one of which is a transcription factor, Nux is the amino-terminal subdomain of a wild-type ubiquitin or a reduced-associating mutant ubiquitin amino-terminal subdomain, Cub is the carboxy-terminal subdomain of a wild-type ubiquitin, X is an amino acid other than methionine and RM is an active reporter moiety; and detecting the degree of cleavage by a ubiquitin-specific protease of the first fusion protein between Cub and X by detecting the degree of the activity of RM, wherein an increase of cleavage is indicative of P1/P2 binding.

In a related aspect, the invention provides a method of determining whether two proteins bind to each other, at least one of which is a membrane-associated protein, comprising the steps of: translationally providing a first fusion protein comprising segments P1, Cub-X, and RM, in an order wherein Cub-X is closer to the N-terminus of the first fusion protein than RM, and a second fusion protein comprising segments Nux and P2, wherein P1 and P2 are proteins, at least one of which is membrane-associated, which proteins may be the same or different, Nux is the amino-terminal subdomain of a wild-type ubiquitin or a reduced-associating mutant ubiquitin amino-terminal subdomain, Cub is the carboxy-terminal subdomain of a wild-type ubiquitin, X is an amino acid and RM is an enzymatically active reporter moiety; and detecting the degree of cleavage by a ubiquitin-specific protease of the first fusion protein between Cub and X by detecting the degree of the enzymatic activity of RM, wherein an increase of cleavage is indicative of P1/P2 binding.

In a related aspect, the invention provides a method of determining whether two proteins bind to each other, at least one of which is a transcription factor, comprising the steps of: translationally providing a first fusion protein comprising segments P1, Cub-X, and RM, in an order wherein Cub-X is closer to the N-terminus of the first fusion protein than RM, and a second fusion protein comprising segments Nux and P2, wherein P1 and P2 are proteins, at least one of which is a transcription factor, Nux is the amino-terminal subdomain of a wild-type ubiquitin or a reduced-associating mutant ubiquitin amino-terminal subdomain, Cub is the carboxy-terminal subdomain of a wild-type ubiquitin, X is an amino acid and RM is an enzymatically active reporter moiety; and, detecting the degree of cleavage by a ubiquitin-specific protease of the first fusion protein between Cub and X by detecting the degree of the enzymatic activity of RM, wherein an increase of cleavage is indicative of P1/P2 binding.

In one embodiment, X is selected from the group consisting of Arginine, Lysine, Histidine, Phenylalanine, Tryptophan, Tyrosine, Leucine, Aspartate, Glutamate, Cysteine, Asparagine, Glutamine and Isoleucine. In a related embodiment, X is Methionine, Glycine or Valine.

In one embodiment, the reporter moiety is selected from the group consisting of: a transcription factor and a fluorescent marker.

In one embodiment, the translationally providing step is performed by a cell that expresses the ubiquitin-specific protease.

In one embodiment, the translationally providing step and the step wherein cleavage between Cub and X may occur is performed by a cell that expresses the ubiquitin-specific protease.

The cell can be a eukaryotic cell, or a mammalian cell, or a fungal cell, or a plant cell, or an insect cell. In certain embodiments, the cell is selected from the group consisting of: a human cell, a mouse cell, a rat cell, a hamster cell, a zebrafish cell, a Drosophila cell, a nematode cell, an S. pombe cell and an S. cerevisiae cell. In another embodiment, the cell is selected from the group consisting of: an A. thaliana cell and an N. tabacum cell.

In one embodiment, the reporter moiety is a negative selectable marker, and the degree of activity of the reporter moiety is determined by incubating the cell under conditions that select against the negative selectable marker so that continued viability of the cell under negative selection conditions indicates that P1 binds P2. In a preferred embodiment, the negative selectable marker is selected from the group consisting of: URA3, Tk, codA, HygTk, Tkneo, TkBSD, PACTk, HygCoda, Codaneo, CodaBSD, PACCoda, HPRT and GPT2. In another preferred embodiment, the negative selectable marker is selected from the group consisting of: TRP1, CYH2, and CAN1.

In one embodiment, the reporter moiety is a positive selectable marker, and the presence or absence of the reporter moiety is determined by comparing the viability of the cell under conditions that select for the positive selectable marker to the viability of the cell under nonselective conditions, so that decreased viability of the cell grown under the positive selection conditions as compared to the viability of the cell grown under the nonselective conditions indicates that P1 binds P2. In a preferred embodiment, the positive selectable marker is selected from the group consisting of: URA3, Tk, codA, HygTk, Tkneo, TkBSD, PACTk, HygCoda, Codaneo, CodaBSD, PACCoda, and GPT2. In another preferred embodiment, the positive selectable marker is selected from the group consisting of: HIS3, LYS2, LEU2, TRP2, ADE2.

In one embodiment, Nux contains at least one point mutation at amino acid 3 or amino acid 13 of a ubiquitin.

In another aspect, the invention provides a method of determining whether a test compound agonizes or antagonizes the binding of two proteins to each other comprising the steps of: translationally providing a first fusion protein comprising segments P1, Cub-X, and RM, in an order wherein Cub-X is closer to the N-terminus of the first fusion protein than RM, and a second fusion protein comprising segments Nux and P2, wherein P1 and P2 are proteins, which proteins may be the same or different, Nux is the amino-terminal subdomain of a wild-type ubiquitin or a reduced-associating mutant ubiquitin amino-terminal subdomain, Cub is the carboxy-terminal subdomain of a wild-type ubiquitin, X is an amino acid other than methionine and RM is an active reporter moiety; and, comparing the amount of cleavage by a ubiquitin-specific protease between Cub and X by detecting the degree of the activity of RM in the presence of the compound with the amount of such cleavage that is expected in the absence of the test compound or in the presence of a standard compound, wherein increased cleavage indicates the test compound is an agonist and decreased cleavage indicates the test compound is an antagonist of P1/P2 binding.

In a related aspect, the invention provides a method of determining whether a test compound agonizes or antagonizes the binding of two proteins to each other comprising the steps of: translationally providing a first fusion protein comprising segments P1, Cub-X, and RM, in an order wherein Cub-X is closer to the N-terminus of the first fusion protein than RM, and a second fusion protein comprising segments Nux and P2, wherein P1 and P2 are proteins, which proteins may be the same or different, Nux is the amino-terminal subdomain of a wild-type ubiquitin or a reduced-associating mutant ubiquitin amino-terminal subdomain, Cub is the carboxy-terminal subdomain of a wild-type ubiquitin, X is an amino acid and RM is an enzymatically active reporter moiety; and, comparing the amount of cleavage by a ubiquitin-specific protease between Cub and X by detecting the degree of the enzymatic activity of RM in the presence of the compound with the amount of such cleavage that is expected in the absence of the test compound or in the presence of a standard compound, wherein increased cleavage indicates the test compound is an agonist and decreased cleavage indicates the test compound is an antagonist of P1/P2 binding.

In another aspect, the invention provides a method for selecting an agonist or antagonist of P1/P2 binding from a library of test compounds, a multiplicity of said library compounds having no known agonist or antagonist activity for P1/P2 binding, comprising: 1) determining the agonist or antagonist activity of each test compound of the library according to the method of claim 40 or 41; and, 2) selecting from the multiplicity at least one test compound that shows agonistic or antagonistic activity.

In one embodiment, the invention provides a method further comprising: selecting a candidate compound from a library of candidates which comprise 2 to 10, 10 to 500, 500 to 10,000 or greater than 10,000 compounds, wherein multiple members of said library are not known to bind P1 or P2. In a preferred embodiment, said library of candidate compounds is selected from the group: synthetic chemical library and natural chemical library.

In one embodiment, the candidate compound is a polypeptide. In a preferred embodiment, said polypeptide is supplied by a polypeptide library. In a preferred embodiment, the candidate compound is a small molecule compound.

In one embodiment, X is selected from the group consisting of: Arginine, Lysine, Histidine, Phenylalanine, Tryptophan, Tyrosine, Leucine, Aspartate, Glutamate, Cysteine, Asparagine, Glutamine and Isoleucine. In another embodiment, X is Methionine, Glycine or Valine.

In one embodiment, the reporter moiety is a selectable marker.

In one embodiment, the selectable marker is selected from the group consisting of: URA3, HIS3, LYS2, HygTk, Tkneo, TkBSD, PACTk, HygCoda, Codaneo, CodaBSD, PACCoda, Tk, codA, HPRT, and GPT2. In another embodiment, the selectable marker is selected from the group consisting of: TRP1, CYH2, and CAN1.

In one embodiment, the reporter moiety is selected from the group consisting of: a transcription factor and a fluorescent marker.

In one embodiment, the translationally providing step is performed by a cell that expresses the ubiquitin-specific protease. In a preferred embodiment, the translationally providing step and the step wherein cleavage between Cub and X may occur is performed by a cell that expresses the ubiquitin-specific protease.

In one embodiment, the cell is a eukaryotic cell, or a mammalian cell, or a fungal cell, or a plant cell, or an insect cell. In another embodiment, the cell is selected from the group consisting of: a human cell, a mouse cell, a rat cell, a hamster cell, a zebrafish cell, a Drosophila cell, a nematode cell, an S. pombe cell and an S. cerevisiae cell. In another embodiment, the cell is selected from the group consisting of: an A. thaliana cell and an N. tabacum cell.

In one embodiment, Nux contains at least one point mutation at amino acid 3 or amino acid 13 of a ubiquitin.

In another aspect, the invention provides a method of characterizing the sequence of a protein that binds a target protein comprising the steps of: expressing a first and a second nucleic acid in a ubiquitin-specific protease expressing cell, which first nucleic acid encodes a target fusion protein comprising segments P1, Cub-X, and RM, in an order wherein Cub-X is closer to the N-terminus of the target fusion protein than RM, wherein P1 is the target protein, Cub is the carboxy-terminal subdomain of a wild-type ubiquitin, X is an amino acid selected from the group consisting of arg, lys, phe, leu, trp, his, asp, asn, tyr, ile, glu, cys and gln, and RM is an enzymatically active reporter moiety, which second nucleic acid encodes a candidate fusion protein comprising segments P2 and Nux, wherein the second nucleic acid is a member of a library containing multiple different nucleic acids differing in the P2 segments they encode, P2 is a candidate segment and Nux is the amino-terminal subdomain of a wild-type ubiquitin or a reduced-associating mutant ubiquitin amino-terminal subdomain; recovering a clone of the cell expressing the first and second nucleic acid under conditions wherein a cell is selectable only in the absence of the enzymatic activity of RM; and, characterizing the second nucleic acid encoding P2.

In one embodiment, the enzymatically active reporter moiety is a negative selectable marker selected from the group consisting of: URA3, Tk, codA, HygTk, Tkneo, TkBSD, PACTk, HygCoda, Codaneo, CodaBSD, PACCoda, HPRT, and GPT2. In another embodiment, the enzymatically active reporter moiety is a negative selectable marker selected from the group consisting of: TRP1, CAN1, and CYH2.

In another aspect, the invention provides a method of characterizing the sequence of a protein that binds a target protein comprising the steps of: expressing a first and a second nucleic acid in a ubiquitin-specific protease expressing cell, which first nucleic acid encodes a target fusion protein comprising segments P1, Cub-X, and RM, in an order wherein Cub-X is closer to the N-terminus of the target fusion protein than RM, wherein P1 is the target protein, Cub is the carboxy-terminal subdomain of a wild-type ubiquitin, X is an amino acid selected from the group consisting of arg, lys, phe, leu, trp, his, asp, asn, tyr, ile, glu, cys and gln, and RM is an active reporter moiety, which second nucleic acid encodes a candidate fusion protein comprising segments P2 and Nux, wherein the second nucleic acid is a member of a library containing multiple different nucleic acids differing in the P2 segments they encode, P2 is a candidate segment and Nux is the amino-terminal subdomain of a wild-type ubiquitin or a reduced-associating mutant ubiquitin amino-terminal subdomain; recovering a clone of the cell expressing the first and second nucleic acid under conditions wherein a cell is selectable only in the absence of an activity of RM; and, characterizing the second nucleic acid encoding P2.

In one embodiment, the active reporter moiety is selected from the group consisting of: a transcription factor and a fluorescent marker.

In one embodiment, the cell is a eukaryotic cell, or a mammalian cell, or a fungal cell, or a plant cell, or an insect cell. In another embodiment, the cell is selected from the group consisting of: a human cell, a mouse cell, a rat cell, a hamster cell, a zebrafish cell, a Drosophila cell, a nematode cell, an S. pombe cell and an S. cerevisiae cell. In another embodiment, the cell is selected from the group consisting of: an A. thaliana cell and an N. tabacum cell.

In one embodiment, the library of nucleic acids comprises 2 to 10, 10 to 500, 500 to 10,000 or greater than 10,000 members, wherein fusions proteins encoded by multiple members of said library are not known to bind P1.

In one embodiment, Nux contains at least one point mutation at amino acid 3 or amino acid 13 of a ubiquitin.

In another aspect, the invention provides a kit for characterizing the sequence of a polypeptide that binds a target protein, which comprises: a first nucleic acid encoding a target fusion protein comprising a cloning site suitable for the insertion of a nucleic acid encoding a target protein sequence, segments Cub-X, and RM, in an order wherein Cub-X is closer to the N-terminus of the target fusion protein than RM, wherein Cub is the carboxy-terminal subdomain of a wild-type ubiquitin, X is an amino acid selected from the group consisting of arg, lys, phe, leu, trp, his, asp, asn, tyr, ile, glu, cys and gln, and RM is an active reporter moiety, which activity allows for selection, whereby a fusion protein comprising the target protein sequence, Cub-X and RM can be expressed; a second nucleic acid comprising an Nux segment encoding the amino-terminal subdomain of a wild-type ubiquitin or a reduced-associating mutant ubiquitin amino-terminal subdomain and a cloning site suitable for the insertion of a nucleic acid encoding a polypeptide sequence whereby a fusion protein comprising Nux and the polypeptide sequence can be expressed; and, instructions indicating that a nucleic acid encoding a defined target protein sequence is to be inserted into the first nucleic acid and members of a library of nucleic acids encoding candidate polypeptides are to be inserted into the second nucleic acid, in order to characterize a polypeptide that binds to the target protein.

In one embodiment, the active reporter moiety is a negative selectable marker selected from the group consisting of: URA3, Tk, codA, HygTk, Tkneo, TkBSD, PACTk, HygCoda, Codaneo, CodaBSD, PACCoda, HPRT, and GPT2. In another embodiment, the active reporter moiety is a negative selectable marker selected from the group consisting of: TRP1, CAN1, and CYH2. In another embodiment, the active reporter moiety is selected from the group consisting of: a transcription factor and a fluorescent marker.

In one embodiment, Nux contains at least one point mutation at amino acid 3 or amino acid 13 of a ubiquitin.

In one embodiment, the expression of first and second nucleic acids are carried out in a cell. The cell can be a eukaryotic cell, or a mammalian cell, or a fungal cell, or a plant cell, or an insect cell. In another embodiment, the cell is selected from the group consisting of: a human cell, a mouse cell, a rat cell, a hamster cell, a zebrafish cell, a Drosophila cell, a nematode cell, an S. pombe cell and an S. cerevisiae cell. In another embodiment, the cell is selected from the group consisting of: an A. thaliana cell and an N. tabacum cell.

In one embodiment, said instructions indicate that the library may comprise 2 to 10, 10 to 500, 500 to 10,000 or greater than 10,000 members, wherein candidate polypeptides encoded by multiple members of said library are not known to bind said defined target protein.

In another aspect, the invention provides a kit for characterizing the sequence of a polypeptide that binds a target protein, which comprises: a first nucleic acid encoding a target fusion protein comprising a cloning site suitable for the insertion of a nucleic acid encoding a target protein sequence, segments Cub-X, and RM, in an order wherein Cub-X is closer to the N-terminus of the target fusion protein than RM, wherein Cub is the carboxy-terminal subdomain of a wild-type ubiquitin, X is an amino acid selected from the group consisting of arg, lys, phe, leu, trp, his, asp, asn, tyr, ile, glu, cys and gln, and RM is an active reporter moiety, which activity allows for selection, whereby a fusion protein comprising the target protein sequence, Cub-X and RM can be expressed; a library of second nucleic acids each comprising an Nux segment encoding the amino-terminal subdomain of a wild-type ubiquitin or a reduced-associating mutant ubiquitin amino-terminal subdomain and a nucleic acid encoding a polypeptide sequence, whereby a library of fusion proteins comprising Nux and the polypeptide sequences can be expressed.

In one embodiment, the invention provides a kit further comprising instructions indicating that a nucleic acid encoding a defined target protein sequence is to be inserted into the first nucleic acid, in order to characterize a polypeptide that binds to the target protein.

In one embodiment, the active reporter moiety is a negative selectable marker selected from the group consisting of: URA3, Tk, codA, HygTk, Tkneo, TkBSD, PACTk, HygCoda, Codaneo, CodaBSD, PACCoda, HPRT, and GPT2. In another embodiment, the active reporter moiety is a negative selectable marker selected from the group consisting of: TRP1, CAN1, and CYH2. In another embodiment, the active reporter moiety is selected from the group consisting of: a transcription factor and a fluorescent marker.

In one embodiment, Nux contains at least one point mutation at amino acid 3 or amino acid 13 of a ubiquitin.

In one embodiment, the expression of first and second nucleic acids are carried out in a cell. The cell can be a eukaryotic cell, or a mammalian cell, or a fungal cell, or a plant cell, or an insect cell. In another embodiment, the cell is selected from the group consisting of: a human cell, a mouse cell, a rat cell, a hamster cell, a zebrafish cell, a Drosophila cell, a nematode cell, an S. pombe cell and an S. cerevisiae cell. In another embodiment, the cell is selected from the group consisting of: an A. thaliana cell and an N. tabacum cell.

In one embodiment, said library comprises 2 to 10, 10 to 500, 500 to 10,000 or greater than 10,000 members, wherein candidate polypeptides encoded by multiple members of said library are not known to bind said defined target protein.

### 3. BRIEF DESCRIPTION OF THE FIGURES

FIG. 1. The split-Ubiquitin technique and its application to the analysis of membrane proteins using a metabolic marker. The carboxy-terminal part of ubiquitin (C_{ub}), fused to the amino-terminus of Ura3p displaying an arginine(R) as its first amino acid (C_{ub}-RUra3p) was linked to the C terminus of Sec63p, and the amino-terminal part of ubiquitin (N_{ub}) was linked to the N terminus of the membrane protein P1. Pathway 1: N_{ub} is coupled to a protein that binds to Sec63p. The complex brings N_{ub} and C_{ub} into close proximity. N_{ub} and C_{ub} reconstitute the quasi-native Ub that is cleaved by the Ub-specific proteases to release RUra3p from C_{ub}. The cleaved RUra3p is targeted for rapid destruction by the enzymes of the N-end rule (3) to yield cells that are uracil auxotrophs and 5-FOA resistant. Pathway 2: N_{ub} is linked to a protein that does not bind to Sec63p. The two fusion proteins do not improve the reconstitution of N_{ub} and C_{ub} into the quasi-native Ub. Thus, RUra3p stays linked to Sec63-C_{ub}, and the cells are uracil prototrophs and 5-FOA sensitive.

FIG. 2. N_{ub} and C_{ub} fusions. (A) N_{ub} (residues 1-36 of Ub) was fused to the N terminus of either a transmembrane protein (constructs 1-11) or a cytosolic protein (constructs 12-13). The N termini of all proteins are located in the cytosol. The orientation and the numbers of the membrane-spanning domains were obtained from published studies. The orientation of the N and the C terminus of Ste14p and its subcellular localization was a subject of this study. The N_{ub}-attached proteins of constructs 1-5 are localized in the ER (Deshaies and Schekman, 1990; Shim et al., 1991; Finke et al., 1996; Wilkinson et al., 1996; Ballensiefen et al., 1998). The localization of the N_{ub}-attached protein of construct 6 was a subject of this study. The N_{ub}-attached protein of construct 7 resides in the early Go1gi and of construct 8 in the late Golgi/plasma membrane (Protopopov et al., 1993; Banfield et al., 1994). The N_{ub}-attached protein of construct 9 was shown to be in the plasma membrane (Aalto et al., 1993). The N_{ub}-attached protein of construct 10 was found in the vacuole, and the N_{ub}-attached protein of construct 11 was found in the outer membrane of the mitochondrion (Kiebler et al., 1993; Darsow et al., 1997; Wada et al., 1997; Srivastava and Jones, 1998). (B) C_{ub} (residues 35-76 of Ub) was linked to the C terminus of a transmembrane protein and extended at its own C terminus by a reporter protein. The C termini of all proteins are localized in the cytosol. The information on the orientation of the N- and C-termini, the numbers of the membrane-spanning domains, and the localization of the unmodified proteins were obtained from published studies except for construct 15, where the number of membrane-spanning domains is still tentative. The C_{ub}-attached protein of construct 14 is localized in the ER, that of construct 16 is found in the plasma membrane, and that of construct 17 is localized in the outer membrane of the mitochondrion (Jund et al., 1988; Feldheim et al., 1992; Moczko et al., 1997). The reporter (R) is RUra3p for the constructs 15-17 and RUra3p or DHFRha (Dha) for construct 14.

FIG. 3. Split-Ub monitors the interaction between Sec63p and Sec62p in vivo. (A) Immunoblot analysis of cells expressing Sec63-C_{ub}-Dha together with an empty plasmid (lane a) or together with N_{ub}-, Nᵤₐ-, or N_{ug}-Sec62p (lanes b, c, and d, respectively) or N_{ub}-, Nᵤₐ-, or N_{ug}-Bos1p (lanes e, f, and g, respectively). The nitrocellulose membrane was probed with the anti-ha antibody that recognizes the uncleaved C_{ub} fusion and the cleaved Dha. (B) Growth assay of the interaction between Sec63p and Sec62p based on split-Ub and a short-lived Ura3p (RUra3p) as a reporter. Sec63CRUp-containing cells bearing either the UBR1 gene or a UBR1 deletion were transformed with an empty plasmid or N_{ub}-, Nᵤₐ-, or N_{ug}-Sec⁶²p. Cells -were pregrown in selective media containing uracil. Cells (10³ or 10²) were spotted on selective plates lacking uracil and also lacking leucine and tryptophan to select for the presence of the C_{ub}- and N_{ub}-constructs.

FIG. 4. The measured proximity between Sec62p and Sec63p is due to both proteins being in one complex. (A) Cells bearing Sec63CRUp and N_{ug}-Sec6²p were transformed with a plasmid containing either Sec62p, Sec62Dha, Ste14Dha, Tpi1ha, or an empty plasmid, all under the control of the P_{GAL1}-promoter (lanes a-e). Approximately 10⁵, 10⁴, 10³, and 10² cells were spotted on selective media lacking uracil and containing either glucose to repress or galactose to induce the P_{GAL1} promoter. (B) S. cerevisiae cells (10⁴) were plated as described in panel A on selective media containing galactose and lacking uracil, and colonies were counted after 4 d. The average of seven independent experiments is shown. Approximately 800 colonies were recovered upon overexpression of Sec62p. This number was arbitrarily set as 100. (C) Overexpression of the ha epitope-bearing proteins was confirmed by immunoblot analysis of extracts of S. cerevisiae cells coexpressing Sec63CRUp, N_{ug}-Sec62p, and the following constructs: Tpi1ha (lanes a and f), Ste14Dha (lanes b and g), Sec62Dha (lanes c and h), Sec62p (lanes d and i), and empty vector (lanes e and j). Cells were grown in glucose (lanes a-e) to repress and grown in galactose (lanes f-j) to induce the expression of the proteins.

FIG. 5. Split Ub measures the proximity between Sec63p and membrane-associated proteins in vivo. Sec63CRUp containing cells expressing N_{ub}, Nᵤₐ, and N_{ug} constructs of Sec62p (A), Sec61p (B), Ssh1p (C), Bos1p (D), Ste14p (E), Sed5p (F), Sso1p (G), Snc1p (H), Tom22p(I), Vam3p (J), Tpi1p (K), and Guk1p (L) were spotted (10⁵ and 10³ cells) on selective media lacking uracil (A-M) and leucine and histidine (A and D) or leucine and tryptophan (B, C, and E-M) to select for the presence of the C_{ub} and N_{ub} constructs. (M) Sec63CRUp-containing cells bearing either the empty plasmid, N_{ub}-, Nᵤₐ-, -N_{ug}-Sec²²p or N_{ub}-, Nᵤₐ-, N_{ug}-Sec⁶¹p were spotted (10⁵, 10⁴, 10³ cells) on plates lacking uracil. Cells were grown for 4 d.

FIG. 6. (A) N_{ub} and C_{ub} constructs of Ste14p are functional. N_{ub}-Ste41p and Ste14CRUp were expressed in cells containing a STE14 deletion and mated with an appropriate tester strain of the opposite mating type. The mated cells were patched on media selecting for the formation of diploids. (B) Ste14p is located between Bos1p and Sed5p. Sec63CRUp containing cells expressing Nᵥ₁-Sec62p (a),-Ssh1p (b),-Bos1p (c),-Ste14p (d),-Sed5p (e),-Sso1p (f), and -Snc1p (g) were spotted (10⁵, 10⁴, 10³, and 10² cells) on SD-ura plates that also lacked leucine and tryptophan to select for the presence of the C_{ub} and Nᵥᵢ constructs. Cells were grown for 3 d. (C) Sec62p, Ssh1p, and Sec61p are equidistant to Ste14p. Ste14CRUp-containing cells expressing N_{ub}, Nᵤₐ, and N_{ug} constructs of Sec62p (a), Ssh1p (b), Sec61p (c), Ste14p (d), Sed5p (e), and Sso1p (f) were spotted (10⁵, 10³, and 10² cells) on selective media lacking uracil, leucine, and tryptophan and containing 500 μM methionine to reduce the expression of Ste14CRUp. Cells were grown for 3 d.

FIG. 7. Tom22p is close to Tom20p; Sso1p and Snc1p are close to Fur4p. (A) Tom2OCRUp-containing S. cerevisiae cells expressing the N_{ub} and Nᵤₐ constructs of Tom22p (a), Sec62p (b), Sso1p (c), and Vam3p (d) were spotted (10³ and 10² cells) on selective media lacking uracil. Cells were grown for 3 d. (B) Fur4CRUp containing S. cerevisiae cells expressing the N_{ub} and Nᵤₐ constructs of Sso1p (a), Snc1p (b), Sec62p (c), and Sed5p (d) were spotted (10⁵ and 10³ cells) on selective media lacking uracil. Cells were grown for 3 d. (C) Tom20CRUp-containing cells bearing the UBR1 gene or a UBR1 deletion were transformed with a plasmid harboring N_{ub}-Tom22p or the empty vector pRS314. Cells (10³ and 10²) were spotted on selective media lacking uracil. Plates were incubated for 3 d.

FIG. 8. A system to select for protein interactions in vivo. (A) The split-ubiquitin system. Ubiquitin, fused to the N terminus of Ura3p displaying an arginine as its first amino acid (RUra3p) is recognized by the UBPs (line 1). The cleaved RUra3p is rapidly degraded by the N-end rule pathway of protein degradation (line 4). No cleavage of RUra3p takes place if only the C_{ub} is fused between Gal4p and RUra3p (line 2). A protein P1 is attached to the N-terminal half of ubiquitin. If P1 interacts with Gal4p, the two coupled Ub peptides are forced into close proximity, a ubiquitin-like molecule is reconstituted, and cleavage by the UBPs is observed (line 3). The freed RUra3p reporter is now rapidly degraded by the enzymes of the N-end rule, resulting in uracil auxotrophy and FOA resistance (line 4). (B) Gal4p interacts with Gal80p in vivo. Shown are serial dilutions of cells coexpressing N_{ub} or a N_{ub}-Gal80p fusion together with Gal4(1-147+768-881)-C_{ub}-RUra3p on plates lacking tryptophan and leucine (Top), additionally lacking uracil (Middle), or containing FOA (Bottom). All proteins were expressed from single-copy vectors. (C) Tup1p interacts with Ssn6p in vivo. Shown are serial dilutions of cells coexpressing the depicted N_{ub} and C_{ub} fusions on plates lacking tryptophan and leucine (Upper) or on plates additionally lacking uracil (Lower). All proteins were expressed from single-copy vectors.

FIG. 9. Nhp6B was isolated in two independent split-ubiquitin screens using Gal4p or Tup1p as C_{ub}-RUra3 baits. (A) Gal4p interacts with Nhp6B in vivo. Serial dilutions of cells coexpressing N_{ub} or an N_{ub}-Nhp⁶B fusion together with a fusion of the DNA-binding and activation domains of Gal4(1-147+768-881)p to C_{ub}-RUra3p were grown on plates lacking tryptophan and leucine (Top), on plates additionally lacking uracil (Middle), or on plates containing FOA (Bottom). N_{ub} and N_{ub} fused to full-length Nhp6B were expressed from multicopy vectors. (B) The activation domain of Gal4p is sufficient for the interaction with Nhp6B. Serial dilutions of cells coexpressing N_{ub}, N_{ub} fused to the activation domain of Gal4p (amino acids 768-881; N_{ub}-Gal4p), or N_{ub} attached to the large subunit of TFIIA (N_{ub}-Toa1p) together with Nhp6B-C_{ub}-RUra3p were grown on plates lacking tryptophan and leucine (Top), on plates additionally lacking uracil (Middle), or on plates containing FOA (Bottom). N_{ub}, N_{ub}-Gal4p, and N_{ub}-Toa1p were expressed from multicopy vectors. (C) Tup1p interacts with Nhp6B in vivo. Serial dilutions of cells coexpressing the depicted N_{ub} and C_{ub} fusions were grown on plates lacking tryptophan and leucine (Top), on plates additionally lacking uracil (Middle), or on plates containing FOA (Bottom). N_{ub} and the clone isolated from the library expressing N_{ub}-Nhp6B that lacked the first 22 amino acids of Nhp6B were on multicopy vectors. (D) Tup1-C_{ub}-RGFP is located in the nucleus and interacts with N_{ub}-Ssn6p and N_{ub}-Nhp6B. Cells expressing the depicted fusions from single-copy vectors were analyzed under a Leitz fluorescence microscope with phase contrast (Left) and fluorescence (Right).

FIG. 10. Nhp6B interacts with Gal4p and Tup1p in vitro. (A) Gal4p coprecipitates together with Nhp6B from S. cerevisiae extracts. Extracts from S. cerevisiae cells expressing N_{ub} or N_{ub}-Gal4p (amino acids 768-881) from multicopy vectors were incubated with GSTp or GST-Nhp6B purified from E. coli on glutathione beads. Coprecipitated proteins were separated on an SDS gel and visualized on a Western blot with an anti-HA antibody with the help of an HA tag present in the N_{ub} moiety. (B) In vitro translated Gal4p interacts with Nhp6B. The activation domain of Gal4p (amino acids 768-881) was radiolabeled by in vitro translation and incubated with a bacterially purified GSTp or a GST-Nhp6B fusion bound to glutathione beads. Coprecipitated proteins were visualized by autoradiography. A truncated form of the activation domain of Gal4p, migrating faster in the SDS gel, showed no interaction with GST-Nhp6B. (C) Purified Tup1p interacts with purified Nhp6B. A H₆HA-Tup1p fusion was purified on an Ni column and incubated with purified GSTp or GST-Nhp6B on glutathione beads. Coprecipitated H₆HA-Tup1p was visualized on a Western blot with an anti-HA antibody.

FIG. 11. The interaction between Nhp6B and Tup1p is biologically relevant. (A) Nhp6 is necessary for glucose repression of the GAL1 promoter. RNA was prepared from the depicted strains carrying a GAL1-LacZ fusion integrated at the GAL1 locus. JD53 was used as wild-type parental strain (lanes 1 and 4). The ΔNHP6 strain was derived from JD53 that lacks NHP6A and NHP6B (lanes 2 and 5). In the strain ΔNHP6+NHP6 (lanes 3 and 6), NHP6A and NHP6B had been reintegrated into the original loci. Equal amounts of total RNA were loaded as confirmed by ethidium bromide staining (not shown) and background hybridization to the 28 S rRNA (Right). The Northern blot was probed with a LacZ probe (lanes 1-3) and with an ACT1 probe (lanes 4-6). We consistently saw a slight increase in the level of ACT1 mRNA in the ΔNHP6 strain. (B) Nhp6 is not necessary for α2p repression. RNA was prepared from the depicted strains, and the Northern blot was probed with an MFA1 probe (Upper) or with an ACT1 probe (Lower). In lane 1, RNA was isolated from JD52, a MATa strain. In lane 2, RNA was isolated from JD53, which was used as wild-type parental MATα strain. Lane 3 contained RNA from JD53 lacking NHP6A and NHP6B (ΔNHP6). For lane 4, NHP6A and NHP6B had been reintegrated into the original loci (ΔNHP6+NHP6). Lane 5 contained RNA from JD53 lacking TUP1 (ΔTUP1). (C) NHP6 and REG1 deletions are synthetically lethal. Shown are serial dilutions of the depicted S. cerevisiae strains carrying a URA3-marked Nhp6B expression plasmid (YCplac33-NHP6B) on medium lacking or containing FOA.

FIG. 12. A truncated form of Gal4p, which displays an impaired interaction with Nhp6B, results in elevated levels of transcription upon deletion of NHP6. (A) Deleting NHP6 results in increased levels of transcription of a GAL1-LacZ reporter by a truncated form of Gal4p. Strains of the indicated genotype carrying a GAL1-LacZ reporter were transformed with the depicted expression plasmids. Arbitrary units of β-galactosidase activity are shown for the parental NLY2 strain, which lacks GAL4 and GAL80 in lanes 1, 3, and 5. The β-galactosidase activities of NLY2 cells additionally lacking NHP6A and NHP6B are shown in lanes 2, 4, and 6. Cells were grown in liquid glucose medium, and β-galactosidase activity was determined as described (33). Numbers were measured in triplicate, and standard deviations were less than 20%. All Gal4p derivatives were expressed from single-copy vectors. (B) Truncating the minimal activation domain of Gal4p results in decreased interaction with Nhp6B. Serial dilutions of cells coexpressing the depicted N_{ub} and C_{ub} fusions were grown on plates lacking tryptophan and leucine (Top), on plates additionally lacking uracil (Middle), or on plates containing FOA (Bottom). All proteins were expressed from single-copy vectors.

### 4. DETAILED DESCRIPTION OF THE INVENTION

4.1. General

In general the invention provides methods and reagents for the selection/characterization of a protein binding partner of a selected protein. Once detected, the invention further provides methods for monitoring the protein/protein binding partner interaction that can be used to detect agonists and antagonists of the interaction.

In part, the invention is based upon the finding that even transient interactions of cellular proteins can be detected using a novel split-ubiquitin based polypeptide association selection/characterization method. This method has been used to demonstrate, for example, the association of Sec63p with various other yeast membrane proteins which traffic through the endoplasmic reticulum (ER) and the Golgi apparatus or are targeted to the plasma membrane.

The invention is understood to encompass modifications and extensions of the above described examples as follows.

The invention further provides certain fusion proteins including that comprising a P1-Cub-X-RM polypeptide, where P1 is a first polypeptide, Cub is a C-terminal sub-domain of ubiquitin, X is a non-methionine amino acid residue and RM is a reporter moiety wherein the fusion protein is cleavable by a UBP in the presence of an interacting fusion protein comprising segments Nux and P2, such as P2-Nux wherein P2 is a second polypeptide that interacts with P1 and Nux is a wild-type or mutant form of Nub sub-domain of ubiquitin, and said cleavage results in the release of the reporter moiety having the non-methionine amino-terminal amino acid residue X and wherein the activity of said reporter moiety can be detected before and/or after said release. The reporter moiety of these fusion proteins may be a negative selectable marker, a positive selectable marker, a metabolic marker, or a transcription factor. In preferred applications, the reporter is a selectable marker which is capable of both positive and negative selection. For example, the reporter moiety may be chosen from the list of URA3, HIS3, LYS2, HygTk, Tkneo, Tk3SD, PACTk, HygCoda, Codaneo, CodaBSD, PACCoda, Tk, codA, and GPT2. The reporter moiety may also be TRP1, CYH2, CAN1, HPRT, beta-galactosidase or a luciferase. Furthermore, the reporter moiety may also be a fluorescent marker, e.g. gfp, yfp or rfp, a transcription factor, e.g. hTBP1 (human TATA binding protein 1(, or DHFR.

The invention further provides peptide libraries expressed as fusion proteins. Such peptide libraries may be synthetic, natural, random, biased-random, constrained, non-constrained and combinatorial peptide libraries. In certain instances, the peptide libraries are provided by expression of nucleic acid construct(s) encoding the polypeptides. The DNA libraries may be cDNA, random, biased-random, synthetic, genomic or oligonucleotide nucleic acid construct(s) encoding the second polypeptides of the invention.

The invention further provides applications utilizing unique polypeptide fusions such as a fusion protein comprising segments P2 and Nux, wherein Nux is a wild-type or mutant form of the amino-terminal sub-domain of ubiquitin.

The invention further provides methods of detecting the binding of a second protein to a first protein, for example comprising: providing the first protein as a first polypeptide fusion comprising the structure P1-Cub-X-RM polypeptide, where P1 is a first polypeptide, Cub is a C-terminal sub-domain of ubiquitin, X is a non-methionine amino acid residue and RM is a reporter moiety; providing a second fusion protein as a second polypeptide fusion comprising the structure P2-Nux where P2 is a second polypeptide and Nux is a wild-type or mutant form of an amino-terminal sub-domain of ubiquitin; allowing the first polypeptide fusion to come into close proximity with the second polypeptide fusion under conditions wherein if the first protein interacts with the second protein, cleavage of the first fusion protein results in release of the reporter moiety having the non-methionine amino-terminal amino acid residue X; providing conditions that allow the detection of activity of the reporter moiety wherein the presence or absence of a detectable signal from the reporter moiety indicates that the second protein binds the first protein. Other aspects of the present invention utilize fusion polypeptides P1-Cub-X-RM wherein RM is a reporter moiety possessing enzymatic activity, and X is an amino acid.

Certain methods of the invention may be performed in an in vitro or an in vivo format. The in vivo formats may utilize a host cell such as a eukaryotic cell. Suitable eukaryotic cells include a mammalian cell including a human, a mouse, a rat, or a hamster cell; a vertebrate cell including a zebrafish cell; an invertebrate cell, particularly an insect cell such as a Drosophila cell, or a nematode cell; a plant cell (e.g. an A. thaliana cell or an N. tabacum cell), and a fungal cell including an S. pombe or an S. cerevisiae cell. In preferred in vivo embodiments of the method of the invention, the reporter moiety is a negative selectable marker. The reporter may also be a positive selectable marker. The marker may be a metabolic marker, a transcription factor, both a positive and negative selectable marker, a fluorescent marker, or DHFR. The method provides for the use of various non-methionine amino acid residues to be engineered to the presumptive amino terminus of the reporter or selectable marker protein. Preferably, this amino acid is Arginine, however it may also be an other non-methionine amino acid—e.g. Lysine, Histidine, Phenylalanine, Tryptophan, Tyrosine, Leucine, Aspartate, Glutamate, Cysteine, Asparagine, Glutamine or Isoleucine.

The method of the invention provide second polypeptides P2, which may be supplied as synthetic, natural, random, biased-random, constrained, non-constrained and combinatorial peptide libraries. These libraries may be provided by expression of nucleic acid construct(s) encoding said second polypeptides. Preffered embodiments of a method of the invention provides a fusion protein comprising P2 and Nux, wherein the Nux is fused to the N-terminus of the second polypeptide P2 or to the C-terminus of the second polypeptide P2. In certain embodiments, Nux may be inserted into a loop of P2, or P2 inserted inta a loop of Nux.

In further preferred embodiments, the invention provides methods of screening for an agonist or antagonist of the binding of a second protein to a first protein comprising: providing the first protein as a first polypeptide fusion comprising the structure P1-Cub-X-RM polypeptide, where P1 is a first polypeptide, Cub is a C-terminal sub-domain of ubiquitin, X is a non/methionine amino acid residue and RM is a reporter moiety; providing a second fusion protein as a second polypeptide fusion; comprising the structure P2-Nux where P2 is a second polypeptide and Nux is a wild-type or mutant form of an amino-terminal sub-domain of ubiquitin; providing at least one candidate agonist or antagonist; allowing the first polypeptide fusion to come into close proximity with the second polypeptide fusion in the presence of said candidate agonist or antagonist under conditions wherein if the first protein interacts with the second protein, cleavage of the first fusion protein results in release of the reporter moiety having the non-methionine amino-terminal amino acid residue X; providing conditions that allow the detection of activity of the reporter moiety wherein the degree of cleavage of the P1-Cub-X-RM polypeptide as evidenced by a change in the activity of the reporter moiety indicates that the candidate agonist or antagonist affects binding of the second protein with the first protein. The agonist and antagonist screening methods may be performed in any of the abovementioned in vitro or in vivo formats. The candidate agonist or antagonist compound may be a small molecule, a peptide, a polypeptide or a protein. The candidate agonist or antagonist peptide, polypeptide or protein provided by expression of a nucleic acid may be provided by a nucleic acid encoding said peptide, polypeptide or protein. The candidate agonist or antagonist may be provided as synthetic, natural, random, biased-random, constrained, non-constrained and combinatorial peptide libraries. In this aspect of the method of the invention, the candidate agonist or antagonist may be provided by expression of nucleic acid construct encoding said first and/or second polypeptides. The candidate agonist or antagonist may be provided by expression of cDNA, random, biased-random, synthetic, genomic or oligonucleotide nucleic acid construct(s) encoding said first and/or second polypeptides. The Nux may be fused to the N-terminus of the second polypeptide P2, or the Nux may be fused to the C-terminus of the second polypeptide P2. In certain embodiments, Nux may be inserted into a loop of P2, or P2 inserted inta a loop of Nux.

In certain preferred embodiments, the method of the invention allows for screening of various agonist or antagonist compounds, preferably the candidate comprises a library comprising 2 to 10, 10 to 500, 500 to 10000 or greater than 10000 agonists or antagonists.

In another aspect, methods of the invention provide a means of selecting/characterizing a second polypeptide that binds to a first polypeptide, for example, comprising: providing the first polypeptide as a first polypeptide fusion comprising the structure P1-Cub-X-RM polypeptide, where P1 is a first polypeptide fusion, Cub is a C-terminal sub-domain of ubiquitin, X is a non-methionine amino acid residue and RM is a reporter moiety; providing a library of candidate second fusion proteins as second polypeptide fusions comprising the structure P2-Nux where P2 is a second polypeptide and Nux is a wild-type or mutant form of an amino-terminal sub-domain of ubiquitin; allowing the first polypeptide fusion to come into close proximity with the library of candidate second polypeptide fusions under conditions wherein if the first protein interacts with a second protein from the library, cleavage of the first fusion protein results in release of the reporter moiety having the non-methionine amino-terminal amino acid residue X; providing conditions that allow the detection of activity of the reporter moiety wherein the degree of activity of the reporter moiety indicates that the second protein binds the first protein, and characterizing at least one second polypeptide P2 that leads to the presence or absence of said detectable signal.

The libraries of the invention include fusion polypeptides comprises 2 to 10, 10 to 500, 500 to 10000 or greater than 10000. The library may be selected from the group synthetic, natural, random, biased-random, constrained, non-constrained and combinatorial peptide libraries. The method of the invention provides for the use of a library of second polypeptide P2, which is provided by expression of nucleic acid construct(s) encoding said second polypeptide. These libraries may be cDNA, random, biased-random, synthetic, genomic or oligonucleotide nucleic acid construct(s) encoding the second polypeptide. The libraries of the invention include arrays of in-frame second fusion proteins encoded by nucleic acid constructs that would encode for the Nux fused to the N- or C-terminus of the second polypeptide P2.

Also included in the invention are certain therapeutic formulations. For example, small molecule or peptide/polypeptide agonist or antagonist compounds of the invention or derived by the methods of the invention, may be incorporated into a formulation for the treatment of a disease or condition.

4.2. Definitions

The term “agonist”, as used herein, is meant to refer to an agent that mimics or upregulates (e.g. potentiates or supplements) bioactivity of a protein of interest, or an agent that facilitates or promotes (e.g. potentiates or supplements) an interaction among polypeptides or between a polypeptide and another molecule (e.g. a steroid, hormone, nucleic acids, small molecule etc.). An agonist can be a wild-type protein or derivative thereof having at least one bioactivity of the wild-type protein. An agonist can also be a small molecule that upregulates expression of a gene or which increases at least one bioactivity of a protein. An agonist can also be a protein or small molecule which increases the interaction of a polypeptide of interest with another molecule, e.g., a target peptide or nucleic acid.

“Antagonist” as used herein is meant to refer to an agent that downregulates (e.g. suppresses or inhibits) bioactivity of the protein of interest, or an agent that inhibits/suppresses or reduces (e.g. destabilizes or decreases) interaction among polypeptides or other molecules (e.g. steroids, hormones, nucleic acids, etc.). An antagonist can be a compound which inhibits or decreases the interaction between a protein and another molecule, e.g., a target peptide, such as interaction between ubiquitin and its substrate. An antagonist can also be a compound that downregulates expression of a gene of interest or which reduces the amount of the wild type protein present. An agonist can also be a protein or small molecule which decreasaes or inhibits the interaction of a polypeptide of interest with another molecule, e.g., a target peptide or nucleic acid.

The term “allele”, which is used interchangeably herein with “allelic variant” refers to alternative forms of a gene or portions thereof. Alleles occupy the same locus or position on homologous chromosomes. When a subject has two identical alleles of a gene, the subject is said to be homozygous for that gene or allele. When a subject has two different alleles of a gene, the subject is said to be heterozygous for the gene. Alleles of a specific gene can differ from each other in a single nucleotide, or several nucleotides, and can include substitutions, deletions, and/or insertions of nucleotides. An allele of a gene can also be a form of a gene containing mutations.

The term “cell death” or “necrosis”, is a phenomenon when cells die as a result of being killed by a toxic material, or other extrinsically imposed loss of function of a particular essential gene function.

“Biological activity” or “bioactivity” or “activity” or “biological function”, which are used interchangeably, for the purposes herein means a catalytic, effector, antigenic, molecular tagging or molecular interaction function that is directly or indirectly performed by the polypeptides of this invention (whether in its native or denatured conformation), or by any subsequence thereof.

“Cells,” “host cells” or “recombinant host cells” are terms used interchangeably herein. It is understood that such terms refer not only to a particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

“Characterize” as used herein means a detailed study of a polypeptide or a nucleic acid (polynucleotide) encoding a polypeptide to reveal relevant chemical and biological information. This information generally includes one or more, but is not limited to, the following: sequence information for protein and nucleic acid, secondary, tertiary, and quarternary structure information, molecular weight, enzymatic or other activity, isoelectric focusing point, binding affinity to other molecules, binding partners, stability, expression pattern, tissue distribution, subcellular localization, expression regulation, developmental roles, phenotypes of transgenic animals overexpressing or devoid of the polypeptide or nucleic acid, size of nucleic acid, and hybridization property of nucleic acid. A variety of standard cell and molecular biology protocols and methodologies can be used, such as gel electrophoresis, capillary electrophoresis, cloning, restriction enzyme digestion, expression profiling by hybridization, affinity chromatography, HPLC, isoelectric focusing, mass spectrometry, automated sequencing, and the generation of transgenic animals, the details of which can be found in many standard molecular biology laboratory manuals (see below). Techniques employing the hybridization of nucleic acids may, for example, utilize arrayed libraries of nucleic acids, such as oligonucleotides, cDNA or others (See, for example, U.S. Pat. No. 5,837,832)

A “chimeric polypeptide” or “fusion polypeptide” is a fusion of a first amino acid sequence encoding a first polypeptide with a second amino acid sequence defining a domain (e.g. polypeptide portion) foreign to and not substantially homologous with any domain of the first polypeptide. Such second amino acid sequence may present a domain which is found (albeit in a different polypeptide) in an organism which also expresses the first polypeptide, or it may be an “interspecies”, “intergenic”, etc. fusion of polypeptide structures expressed by different kinds of organisms. At least one of the first and the second polypeptides may also be partially or completely synthetic or random, i.e. not previously identified in any organism.

“To clone” as used herein, as will be apparent to skilled artisan, may be meant as obtaining exact copies of a given polynucleotide molecule using recombinant DNA technology. Details of molecular cloning can be found in a number of commonly used laboratory protocol books such as Molecular Cloning: A Laboratory Manual, 2nd Ed., ed. by Sambrook, Fritsch and Maniatis (Cold Spring Harbor Laboratory Press: 1989).

“To clone” as used herein, as will be apparent to skilled artisan, may be also meant as obtaining identical or nearly identical population of cells possecessing a common given property, such as the presence or absence of a fluorescent marker, or a positive or negative selectable marker. The population of identical or nearly identical cells obtained by cloning is also called a “clone.” Cell cloning methods are well known in the art as described in many commonly available laboratory manuls (see Current Protocols in Cell Biology, CD-ROM Edition, ed. by Juan S. Bonifacino, Jennifer Lippincott-Schwartz, Joe B. Harford, and Kenneth M. Yamada, John Wiley & Sons, 1999).

“Complementation screen” as used herein means genetic screening for genes or source DNA that can conferred certain specified phenotype which will not exist without the presence of said genes or source DNA. It is usually done in vivo, by introducing into cells lacking certain phenotype a library of source DNA to be screened for, and identifying cells that have obtained a source DNA and now exhibit the specified phenotype. Alternatively, it could be done in vivo by randomly inactivating genes in the genome of the cell lacking certain phenotype and identify cells that have lost the function of certain genes and exhibit the specificed phenotype. However, complementation screen can also be done in vitro in cell-free systems, either by testing each candidate individually or as pools of individuals.

“Recovering a clone of the cell . . . under conditions wherein a cell is selectable” as used herein is meant as selecting from a population of cells, a subpopulation or a single cell possessing a common given property such as the presence or absence of fluorescent markers, or the presence or absence of positive or negative selectable markers, and obtaining a clone of each selected cell. The cells can be selected under conditions that will completely or nearly completely eliminate any cell that does not have the desired property of the cells to be selected. For example, by growing cells in selective media, only cells possessing a certain desired property will survive. The surviving cells can be cloned using standard cell and molecular biology protocols (see Current Protocols in Cell Biology, CD-ROM Edition, ed. by Juan S. Bonifacino, Jennifer Lippincott-Schwartz, Joe B. Harford, and Kenneth M. Yamada, John Wiley & Sons, 1999). Alternatively, cells possessing a desired property can be selected from a population based on the observation of a certain discernable phenotype, such as the presence or absence of fluoresent markers. The selected cells can then be cloned using standard cell and molecular biology protocols (see Current Protocols in Cell Biology, CD-ROM Edition, ed. by Juan S. Bonifacino, Jennifer Lippincott-Schwartz, Joe B. Harford, and Kenneth M. Yamada, John Wiley & Sons, 1999).

The term “equivalent” is understood to include polypeptides or nucleotide sequences that are functionally equivalent or possess an equivalent activity as compared to a given polypeptide or nucleotide sequence. Equivalent nucleotide sequences will include sequences that differ by one or more nucleotide substitutions, additions or deletions, such as allelic variants; and will, therefore, include sequences that differ from the nucleotide sequence of a particular gene, due to the degeneracy of the genetic code. Equivalent polypeptides will include polypeptides that differ by one or more amino acid substitutions, additions or deletions, which amino acid substitutions, additions or deletions leave the function and/or activity of the polypeptide substantially unaltered. A polypeptide equivalent to a given polypeptide could e.g. be the polypeptide that performs the same function in another species. For example, murine ubiquitin herein is considered an equivalent of human ubiquitin.

As used herein, the terms “gene”, “recombinant gene” and “gene construct” refer to a nucleic acid comprising an open reading frame encoding a polypeptide, including both exon and (optionally) intron sequences. The term “intron” refers to a DNA sequence present in a given gene which is not translated into protein and is generally found between exons.

“Homology” or “identity” or “similarity” refers to sequence similarity between two peptides or between two nucleic acid molecules, with identity being a more strict comparison. Homology and identity can each be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences. An “unrelated” or “non-homologous” sequence shares less than 40% identity, though preferably less than 25% identity with another sequence.

The term “interact” as used herein is meant to include detectable interactions (e.g. biochemical interactions) between molecules, such as interaction between protein-protein, protein-nucleic acid, nucleic acid-nucleic acid, and protein-small molecule or nucleic acid-small molecule in nature.

The term “isolated” as used herein with respect to nucleic acids, such as DNA or RNA, refers to molecules separated from other DNAs, or RNAs, respectively, that are present in the natural source of the macromolecule. For example, an isolated nucleic acid encoding one of the subject polypeptides preferably includes no more than 10 kilobases (kb) of nucleic acid sequence which naturally immediately flanks the gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

“Kit” as used herein means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

“Instruction(s)” as used herein means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can contain one or multiple documents or future updates.

“Library” as used herein generally means a multiplicity of member components constituting the library which member components individually differ with respect to at least one property, for example, a chemical compound library. Particularly, as will be apparent to skilled artisan, “library” means a plurality of nucleic acids/polynucleotides, preferrably in the form of vectors comprising functional elements (promoter, transcription factor binding sites, enhancer, etc.) necessary for expression of polypeptides, either in vitro or in vivo, which are functionally linked to coding sequences for polypeptides. The vector can be a plasmid or a viral-based vector suitable for expression in prokaryotes or eukaryotes or both, preferably for expression in mammalian cells. There should also be at least one, preferably multiple pairs of cloning sites for insertion of coding sequences into the library, and for subsequent recovery or cloning of those coding sequences. The cloning sites can be restriction endonuclease recognition sequences, or other recombination based recognition sequences such as loxP sequences for Cre recombinase, or the Gateway system (Life Technologies, Inc.) as described in U.S. Pat. No. 5,888,732, the contents of which is incorporated by reference herein. Coding sequences for polypeptides can be cDNA, genomic DNA fragments, or random/semi-random polynucleotides. The methods for cDNA or genomic DNA library construction are well-known in the art, which can be found in a number of commonly used laboratory molecular biology manuls (see below).

The term “modulation” as used herein refers to both upregulation (i.e., activation or stimulation, e.g., by agonizing or potentiating) and downregulation (i.e. inhibition or suppression e.g., by antagonizing, decreasing or inhibiting) of an activity.

The term “mutation” or “mutated” as it refers to a gene or nucleic acid means an allelic or modified form of a gene or nucleic acid, which exhibits a different nucleotide sequence and/or an altered physical or chemical property as compared to the wild-type gene or nucleic acid. Generally, the mutation could alter the regulatory sequence of a gene without affecting the polypeptide sequence encoded by the wild-type gene. But more commonly, a mutated gene or nucleic acid will either completely lose the ability to encode a polypeptide (null mutation) or encode a polypeptide with an altered property, including a polypeptide with reduced or enhanced biological activity, a polypeptide with novel biological activity, or a polypeptide that interferes with the function of the corresponding wild-type polypeptide. Alternatively, a mutation may take advantage of the degeneracy of the genetic code, by replacing a triplett codon by a different triplett codon that nevertheless encodes the same amino acid as the wild-type triplett codon. Such replacement may, for example, lead to increased stability of the gene or nucleic acid under certain conditions. Furthermore, a mutation may comprise a nucleotide change in a single position of the gene or nucleic acid, or in several positions, or deletions or additions of nucleotides in one or several positions.

The term “reduced-associating mutant” as used herein means a mutant polypeptide that exhibits reduced affinity for its normal binding partner. For example, a reduced-associating mutant of the ubiquitin N-terminus (Nux) is a polypeptide that exhibits reduced affinity for its normal binding partner—the C-terminal half of ubiquitin (Cub), to the point that it will show reduced association or not associate with a wild-type Cub and form a “quasi-wild-type ubiquitin” without the supplemented binding affinity between two polypeptides fused to Nux and Cub, respectively. In a preferred embodiment of the invention, such mutations in Nux are certain missense mutations introduced to either the 3^{rd} or the 13^{th} amino acid residue of the wild-type ubiquitin. Different missense mutations at these positions may differentially affect the affinity/association between Nux and Cub, thereby providing different sensitivity of the assay as disclosed by the instant invention. These missense point mutations can be routinely introduced into cloned genes using standard molecular biology protocols, such as site-directed mutagenesis using PCR.

As used herein, the term “nucleic acid,” in its broadest sense, refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

Specifically, “nucleic acid(s)” may refer to polynucleotides that contain information required for transcription and/or translation of polypeptides encoded by the polynucleotides. These include, but are not limited to, plasmids comprising transcription signals (e.g. transcription factor binding sites, promoters and/or enhancers) functionally linked to downstream coding sequences for polypeptides, genomic DNA fragments comprising transcription signals (e.g. transcription factor binding sites, promoters and/or enhancers) functionally linked to downstream coding sequences for polypeptides, cDNA fragments (linear or circular) comprising transcription signals (e.g. transcription factor binding sites, promoters and/or enhancers) functionally linked to downstream coding sequences for polypeptides, or RNA molecules comprising functional elements for translation either in vitro or in vivo or both, which are functionally linked to sequences encoding polypeptides. These polynecleotides should also be understood to include, as equivalents, analogs of either RNA or DNA made from nucleotide analogs, and, as applicable to the embodiment being described, single (sense or antisense) and double-stranded polynucleotides. These polynucleotides can be in an isolated form, e.g. an isolated vector, or included into the episome or the genome of a cell.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

Databases with individual sequences are described in Methods in Enzymology, ed. Doolittle, supra. Databases include Genbank, EMBL, and DNA Database of Japan (DDBJ). In comparing a new nucleic acid with known sequences, several alignment tools are available. Examples include PileUp, which creates a multiple sequence alignment, and is described in Feng et al., J. Mol. Evol. (1987) 25:351-360. Another method, GAP, uses the alignment method of Needleman et al., J. Mol. Biol. (1970) 48:443-453. GAP is best suited for global alignment of sequences. A third method, BestFit, functions by inserting gaps to maximize the number of matches using the local homology algorithm of Smith and Waterman, Adv. Appl. Math. (1981) 2:482-489.

As used herein, the term “promoter” means a DNA sequence that regulates expression of a selected DNA sequence operably linked to the promoter, and which effects expression of the selected DNA sequence in cells. The term encompasses “tissue specific” promoters, i.e. promoters, which effect expression of the selected DNA sequence only in specific cells (e.g. cells of a specific tissue). The term also covers so-called “leaky” promoters, which regulate expression of a selected DNA primarily in one tissue, but cause expression in other tissues as well. The term also encompasses non-tissue specific promoters and promoters that constitutively express or that are inducible (i.e. expression levels can be controlled).

The terms “protein”, “polypeptide” and “peptide” are used interchangeably herein when referring to a natural or recombinant gene product or fragment thereof which is not a nucleic acid .

The term “recombinant protein” refers to a polypeptide which is produced by recombinant DNA techniques, wherein generally, DNA encoding a polypeptide is inserted into a suitable expression vector which is in turn used to transform a host cell to produce the polypeptide encoded by said DNA. This polypeptide may be one that is naturally expressed by the host cell, or it may be heterologous to the host cell, or the host cell may have been engineered to have lost the capability to express the polypeptide which is otherwise expressed in wild type forms of the host cell. The polypeptide may also be a fusion polypeptide. Moreover, the phrase “derived from”, with respect to a recombinant gene, is meant to include within the meaning of “recombinant protein” those proteins having an amino acid sequence of a native polypeptide, or an amino acid sequence similar thereto which is generated by mutations, including substitutions, deletions and truncation, of a naturally occurring form of the polypeptide.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the methods of the invention.

“Transcription” is a generic term used throughout the specification to refer to a process of synthesizing RNA molecules according to their corresponding DNA template sequences, which may include initiation signals, enhancers, and promoters that induce or control transcription of protein coding sequences with which they are operably linked. “Transcriptional repressor,” as used herein, refers to any of various polypeptides of prokaryotic or eukaryotic origin, or which are synthetic artificial chimeric constructs, capable of repression either alone or in conjunction with other polypeptides and which repress transcription in either an active or a passive manner. It will also be understood that the transcription of a recombinant gene can be under the control of transcriptional regulatory sequences which are the same or which are different from those sequences which control transcription of the naturally-occurring forms of the recombinant gene, or its components.

“Translation” as used herein is a generic term used to describe the synthesis of protein or polypeptide on a template, such as messenger RNA (mRNA). It is the making of a protein/polypeptide sequence by translating the genetic code of an mRNA molecule associated with a ribosome. The whole process can be performed in vivo inside a cell using protein translation machinery of the cell, or be performed in vitro using cell-free systems, such as reticulocyte lysates or any other equivalents. The RNA template for translation may be separately provided either directly as RNA or indirectly as the product of transcription from a provided DNA template, such as a plasmid.

“Translationally providing” means providing a polypeptide/protein by way of translation. As defined above, translation is a process that can be done in vivo inside a cell using protein translation machinery of the cell, or be performed in vitro using cell-free systems, such as reticulocyte lysates or any other equivalents. The RNA template for translation may be separately provided either directly as RNA or indirectly as the product of transcription from a provided DNA template, such as a plasmid. The template DNA can be introduced into a host/target cell by a variety of standard molecular biology procedures, such as transformation, transfection, mating (e.g. add Brent reference WO ???) or cell fusion, or can be provided to an in vitro translation reaction directly.

As used herein, the term “transfection” means the introduction of a nucleic acid, e.g., via an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of a polypeptide or, in the case of anti-sense expression from the transferred gene, the expression of a naturally-occurring form of the polypeptide is disrupted.

As used herein, the term “transgene” means a nucleic acid sequence (encoding, e.g., a polypeptide, or an antisense transcript thereto) which has been introduced into a cell. A transgene could be partly or entirely heterologous, i.e., foreign, to the transgenic animal or cell into which it is introduced, or, homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of the polypeptide, e.g. either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques.

The term “treating” as used herein is intended to encompass curing as well as ameliorating at least one symptom of the condition or disease.

The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The ubiquitins are a class of proteins found in all eukaryotic cells. The ubiquitin polypeptide is characterized by a carboxy-terminal glycine residue that is activated by ATP to a high-energy thiol-ester intermediate in a reaction catalyzed by a ubiquitin-activating enzyme (E1). The activated ubiquitin is transferred to a substrate polypeptide via an isopeptide bond between the activated carboxy-terminus of ubiquitin and the epsilon-amino group of a lysine residue(s) in the protein substrate. This transfer requires the action of ubiquitin conjugating enzymes such as E2 and, in some instances, E3 activities. The ubiquitin modified substrate is thereby altered in biological function, and, in some instances, becomes a substrate for components of the ubiquitin-dependent proteolytic machinery which includes both UBP enzymes as well as proteolytic proteins which are subunits of the proteasome. As used herein, the term “ubiquitin” includes within its scope all known as well as unidentified eukaryotic ubiquitin homologs of vertebrate or invertebrate origin which can be classified as equivalents of human ubiquitin. Examples of ubiquitin polypeptides as referred to herein include the human ubiquitin polypeptide which is encoded by the human ubiquitin encoding nucleic acid sequence (GenBank Accession Numbers: U49869, X04803). Equivalent ubiquitin polypeptide encoding nucleotide sequences are understood to include those sequences that differ by one or more nucleotide substitutions, additions or deletions, such as allelic variants; as well as sequences which differ from the nucleotide sequence encoding the human ubiquitin coding sequence due to the degeneracy of the genetic code. Another example of a ubiquitin polypeptide as referred to herein is murine ubiquitin which is encoded by the murine ubiquitin encoding nucleic acid sequence (GenBank Accession Number: X51730). It will be readily apparent to the person skilled in the art how to modify the methods and reagents provided by the present inevntion to the use of ubiquitin polypeptides other than human ubiquitin.

The term “ubiquitin-like protein” as used herein refers to a group of naturally occurring proteins, not otherwise describable as ubiquitin equivalents, but which nonetheless show strong amino acid homology to human ubiquitin. As used herein this term includes the polypeptides NEDD8, UBL1, NPVAC, and NPVOC. These “ubiquitin-like proteins” are at least over 40% identical in sequence to the human ubiquitin polypeptide and contain a pair of carboxy-terminal glycine residues which function in the activation and transfer of ubiquitin to target substrates as described supra.

As used herein, the term “ubiquitin-related protein” as used herein refers to a group of naturally occurring proteins, not otherwise describable as ubiquitin equivalents, but which nonetheless show some relatively low degree (<40% identity) of amino acid homology to human ubiquitin. These “ubiquitin-related” proteins include human Ubiquitin Cross-Reactive Protein (UCRP, 36% identical to huUb, Accession No. P05161), FUBI (36% identical to huUb, GenBank Accession No. AA449261), and Sentrin/Sumo/Pic1 (20% identical to huUb, GenBank Accession No. U83117). The term “ubiquitin-related protein” as used herein further pertains to polypeptides possessing a carboxy-terminal pair of glycine residues and which function as protein tags through activation of the carboxy-terminal glycine residue and subsequent transfer to a protein substrate.

The term “ubiquitin-homologous protein” as used herein refers to a group of naturally occurring proteins, not otherwise describable as ubiquitin equivalents or ubiquitin-like or ubiquitin-related proteins, which appear functionally distinct from ubiquitin in their ability to act as protein tags, but which nonetheless show some degree of homology to human ubiquitin (34-41% identity). These “ubiquitin-homologous proteins” include RAD23A (36% identical to huUb, SWISS-PROT. Accession No. P54725), RAD23B (34% identical to huUb, SWISS-PROT. Accession No. P54727), DSK2 (41% identical to huUb, GenBank Accession No. L40587), and GDX (41% identical to huUb, GenBank Accession No. J03589). The term “ubiquitin-homologous protein” as used herein is further meant to signify a class of ubiquitin homologous polypeptides whose similarity to ubiquitin does not include glycine residues in the carboxy-terminal and penultimate residue positions. Said proteins appear functionally distinct from ubiquitin, as well as ubiquitin-like and ubiquitin-related polypeptides, in that, consistent with their lack of a conserved carboxy-terminal glycine for use in an activation reaction, they have not been demonstrated to serve as tags to other proteins by covalent linkage.

The term “ubiquitin conjugation machinery” as used herein refers to a group of proteins which function in the ATP-dependent activation and transfer of ubiquitin to substrate proteins. The term thus encompasses: E1 enzymes, which transform the carboxy-terminal glycine of ubiquitin into a high energy thiol intermediate by an ATP-dependent reaction; E2 enzymes (the UBC genes), which transform the E1-S˜Ubiquitin activated conjugate into an E2-S˜Ubiquitin intermediate which acts as a ubiquitin donor to a substrate, another ubiquitin moiety (in a poly-ubiquitination reaction), or an E3; and the E3 enzymes (or ubiquitin ligases) which facilitate the transfer of an activated ubiquitin molecule from an E2 to a substrate molecule or to another ubiquitin moiety as part of a polyubiquitin chain. The term “ubiquitin conjugation machinery”, as used herein, is further meant to include all known members of these groups as well as those members which have yet to be discovered or characterized but which are sufficiently related by homology to known ubiquitin conjugation enzymes so as to allow an individual skilled in the art to readily identify it as a member of this group. The term as used herein is meant to include novel ubiquitin activating enzymes which have yet to be discovered as well as those which function in the activation and conjugation of ubiquitin-like or ubiquitin-related polypeptides to their substrates and to poly-ubiquitin-like or poly-ubiquitin-related protein chains.

The term “ubiquitin-dependent proteolytic machinery” as used herein refers to proteolytic enzymes which function in the biochemical pathways of ubiquitin, ubiquitin-like, and ubiquitin-related proteins. Such proteolytic enzymes include the ubiquitin C-terminal hydrolases, which hydrolyze the linkage between the carboxy-terminal glycine residue of ubiquitin and various adducts; UBPs, which hydrolyze the glycine76-lysine48 linkage between cross-linked ubiquitin moieties in poly-ubiquitin conjugates; as well as other enzymes which function in the removal of ubiquitin conjugates from ubiquitinated substrates (generally termed “deubiquitinating enzymes”). The aforementioned protease activities function in the removal of ubiquitin units from a ubiquitinated substrate following or during uibiquitin-dependent degradation as well as in certain proofreading functions in which free ubiquitin polypeptides are removed from incorrectly ubiquitinated proteins. The term “ubiquitin-dependent proteolytic machinery” as used herein is also meant to encompass the proteolytic subunits of the proteasome (including human proteasome subunits C2, C3, C5, C8, and C9). The term “ubiquitin-dependent proteolytic machinery” as used herein thus encompasses two classes of proteases: the deubiquitinating enzymes and the proteasome subunits. The protease functions of the proteasome subunits are not known to occur outside the context of the assembled proteasome, however independent functioning of these polypeptides has not been excluded.

The term “ubiquitin system” as referred to herein is meant to describe all of the aforementioned components of the ubiquitin biochemical pathways including ubiquitin, ubiquitin-like proteins, ubiquitin-related proteins, ubiquitin-homologous proteins, ubiquitin conjugation machinery, ubiquitin-dependent proteolytic machinery, or any of the substrates which these ubiquitin system components act upon.

4.3. Selectable Reporters for Yeast and Mammalian Cells

The invention provides negative selectable marker genes or “negative selectable reporter moieties” which can be used in a eukaryotic host cell, preferably a yeast or a mammalian cell, and which can be selected against under appropriate conditions. In preferred embodiments, the selectable reporter is provided as a fusion polypeptide with a carboxy- or C-terminal subdomain of ubiquitin (or Cub) and is in some embodiments of the present invention altered so as to encode a non-methionine amino acid residue at the junction with the Cub. The non-methionine amino acid residue is preferably an amino acid which is recognized by the N-end rule ubiquitin protease system (e.g. an arginine, lysine histidine, phenylalanine, tryptophan, tyrosine, leucine or isoleucine residue) and which, when present at the amino-terminal end of the negative selectable marker, targets the negative selectable marker for rapid proteolytic degradation. It will be readily apparent to the person skilled in the art that the choice of amino acid residue recognized by the N-end rule ubiquitin protease system that is optimal for a given host cell depends on the type of host cell used, as, for example, the ubiquitin-dependent proteolytic machinery in yeast cells recognizes a slightly different set of amino acid residues than the ubiquitin-dependent proteolytic machinery in mammalian cells (Varshavsky (1992) Cell 69: 725-35).

A preferred example of a negative selectable marker gene for use in yeast is the URA3 gene which can be both selected for (positive selection) by growing ura3 auxotrophic yeast strains in the absence of uracil, and selected against (negatively selection) by growing cells on media containing 5-fluoroorotic acid (5-FOA) (see Boeke, et al. (1987) Methods Enzymol 154: 164-75). The concentration of 5-FOA can be optimized by titration so as to maximally select for cells in which the URA3 reporter is inactivated by proteolytic degradation to some preferred extent. For example, relatively high concentrations of 5-FOA can be used which allow only cells expressing very low steady-state levels of URA3 reporter to survive. Such cells will correspond to those in which the first and second ubiquitin subdomain fusion proteins have a relatively high affinity for one another, resulting in efficient reassembly of the Nub and Cub fragments and a correspondingly efficient release of the X-URA3 labilized marker. In contrast, lower concentrations of 5-FOA can be used to select for protein binding partners with relatively weak affinities for one another. In addition, proline can be used in the media as a nitrogen source to make the cells hypersensitive to the toxic affects of the 5-FOA (McCusker & Davis (1991) Yeast 7: 607-8). Accordingly, proline concentrations, as well as 5-FOA concentrations can be titrated so as to obtain an optimal selection for URA3 reporter deficient cells. Therefore the use of URA3 as a negative selectable marker allows a broad range of selective stringencies which can be adapted to minimize false positive background noise and/or to optimize selection for high affinity binding interactions. Other negative selectable markers which operate in yeast and which can be adapted to the method of the invention are included within the scope of the invention.

Another example of a negative selectable marker gene for use in yeast is the TRP1 gene which can be both selected for (positive selection) by growing trp1 auxotrophic yeast strains in the absence of tryptophan, and selected against (negatively selection) by growing cells on media containing 5-fluoroanthranilic acid (5-FAA) (Toyn et al. (2000) Yeast 16: 553-560).

Two other negative selectable marker genes for the use in yeast are CYH2 and CAN1 both of which can be selected against (negative selection) by growing cells on media containing cycloheximide or canavanine (The yeast two-hybrid system, ed. by Bartel and Fields, Oxford University Press: 1997).

Numerous selectable markers which operate in mammalian cells are known in the art and can be adapted to the method of the invention so as to allow direct negative selection of interacting proteins in mammalian cells. Examples of mammalian negative selectable markers include Thymidine kinase (Tk) (Wigler et al. (1977) Cell 11: 223-32; Borrelli et al. (1988) Proc. Natl. Acad. Sci. USA 85: 7572-76) of the Herpes Simplex virus, the human gene for hypoxanthine phosphoriboxyl transferase (HPRT) (Lester et al. (1980) Somatic Cell Genet. 6: 241-59; Albertini et al. (1985) Nature 316: 369-71) and Cytidine deaminase (codA) from E. coli (Mullen et al. (1992) Proc. Natl. Acad. Sci. USA 89: 33-37; Wei and Huber (1996) J. Biol. Chem. 271: 3812-16). For example: the Tk gene can be selected against using Gancyclovir (GANC) (e.g. using a 1 uM concentration) and codA gene can be selected against using 5-Fluor Cytidin (5-FIC) (e.g. using a 0.1-1.0 mg/ml concentration). In addition, certain chimeric selectable markers have been reported (Karreman (1998) Gene 218: 57-61) in which a functional mammalian negative selectable marker is fused to a functional mammalian positive selectable marker such as Hygromycinresistance (Hyg^{R}, neomycin resistance (neo^{R}), puromycin resistance (PAC^{R}) or Blasticidin S resistance (BlaS^{R}). These produce various Tk-based positive/negative selectable markers for mammalian cells such as HygTk, Tkneo, TkBSD, and PACTk, as well as various codA-based positive/negative selectable markers for mammalian cells such as HygCoda, Codaneo, CodaBSD, and PACCoda. Tk-neo reporters which incorporate luciferase, green fluorescent protein and/or beta-galactosidase have also been recently reported (Strathdee et al. (2000) BioTechniques 28: 210-14). These vectors have the advantage of allowing ready screening of the “positive” marker/reporter by fluorescent and/or immunofluorescent microscopy. The use of such positive/negative selectable markers affords the advantages mentioned above for URA3 as a reporter in yeast, inasmuch as they allow mammalian cells to be assessed by both positive and negative selection methods for the expression and relative steady-state level of the reporter fusion. For example, Rojo-Niersbach et al reported the use of GPT2 (Guanine Phosphoryl Transferase 2) in mammalian cells as a basis for the selection of protein interactions (Biochem. J. 348: 585-590, 2000).

In certain embodiments, the invention further provides positive selectable marker genes or “positive selectable reporter moieties” which can be used in a eukaryotic host cell, preferably a yeast or a mammalian cell, and which can be selected for under appropriate conditions. In preferred embodiments, the selectable reporter is provided as a fusion polypeptide with a carboxy- or C-terminal subdomain of ubiquitin (or Cub) and is in some embodiments of the present invention altered so as to encode a non-methionine amino acid residue at the junction with the Cub as further described supra. In principle, any non-redundant gene in a synthetic pathway that is essential to the survival of the cell can be used for the construction of an auxotrophic positive selectable marker, but frequently used such makers include, without limitation, HIS3, LYS2, LEU2, TRP2, ADE2. Usually, a cell line is constructed that is deficient in the marker gene, and that can only grow on media supplemented with the corresponding metabolic product, i.e. histidine, lysine, leucine, tryptophane or adenine. When used for selection, a desirable phenotype, i.e. expression of a desired recombinant gene, is linked to the expression of the gene the cell is deficient in by transforming cells with gene constructs comprising both the desired recombinant gene and a recombinant form of the marker gene. Other positive selectable markers include antibiotic resistance markers, e.g. Hygromycinresistance (Hyg^{R}), neomycin resistance (neo^{R}), puromycin resistance (PAC^{R}) or Blasticidin S resistance (BlaS^{R}), as mentioned supra, or any other antibiotic resistance marker. Here, expression of a desired recombinant gene is linked to the expression of the antibiotic resistance marker by transforming cells with gene constructs comprising both the desired recombinant gene and a recombinant form of the antibiotic resistance marker gene. Selection is then carried out on media containing the antibiotic, e.g. Hygromycin, neomycin, puromycin or Blasticidin S. Furthermore, the above mentioned combinations of positive and negative markers can also be employed.

Other advantages of these reporter and selectable marker constructs will be apparent to the skilled artisan.

4.4. Components of N-end Rule Proteolytic Pathway

“N-end rule” system for proteolytic degradation is a particular branch of the ubiquitin-mediated proteolytic pathway present in eukaryotic cells (Bachmair et al. (1986) Science 234: 179-86). This system operates to degrade a cellular polypeptide at a rate dependent upon the amino-terminal amino acid residue of that polypeptide. Protein translation ordinarily initiates with an ATG methionine codon and so most polypeptides have an amino-terminal methionine residue and are typically relatively stable in vivo. For example, in the yeast S. cerevisiae, a beta-galactosidase polypeptide with a methionine amino terminus has a half-life of >20 hours (Varshavsky (1992) Cell 725-35). Under certain circumstances, however, polypeptides possessing a non-methionine amino-terminal residue can be created. For example, when an endoprotease hydrolyzes and thus cleaves a unique polypeptide bond (Y-X) internal to a polypeptide, it results in the release of two separate polypeptides—one of which possesses an amino-terminal amino acid, X, which may not be methionine. For example, the endoprotease UBP, which is a preferred component of the present invention, will cleave a polypeptide bond carboxy-terminal to the final glycine residue (codon 76), regardless of what the next codon is. In the normal function of the cell, this isopeptidase serves to cleave a polyubiquitin precursor into individual ubiquitin units. However it can also be used to generate a target polypeptide with virtually any amino-terminal residue by merely fusing the target polypeptide in-frame to a codon corresponding to the desired amino-terminal amino acid (X), which codon, in turn, is fused downstream of ubiquitin (typically contiguous with ubiquitin Gly codon 76). The resulting target gene chimera construct, has the general structure Ubiquitin-X-Target. Preferred target constructs further comprise an epitope tag (Ep) so that the resulting target gene chimera construct has the general structure Ubiquitin-X-Ep-target, which results in the eventual production of a polypeptide of the general structure X-Ep-Target. Constitutively active UBP activities present in eucaryotic cells will result in the endoproteolytic processing of the Ubiquitin-X-Target polypeptide into Ubiquitin and X-Target entities. The X-Target polypeptide is further acted upon by the components of the N-end rule system as described below. If the Target polypeptide is a negative selection marker (NSM) and if X is an amino acid residue (such as arg) which potentiates rapid degradation by the N-end rule system, then cells expressing intact Ubiquitin-X-NSM can be selected against while cells in which the fusion is clipped into a relatively labile X-NSM polypeptide can be selected for.

It has been determined, with reasonable reliability, the relative effect of a given amino-terminal residue, X, upon target polypeptide stability. For example, when all 20 possible amino-terminal amino acid residues were tested to determine their effect on the stability of beta-galactosidase (utilizing a ubiquitin-X-beta-galactosidase chimeric fusion) in Saccharomyces cerevisiae, drastic differences were discovered (see Varshavsky (1992) Cell 69: 725-35). For example when X was met, cys, ala, ser, thr, gly, val, or pro, the resulting polypeptide was very stable (half-life of >20 hours). When X was tyr, ile, glu, or gln, the resulting polypeptide possessed moderate protein stability (half-life of 10-30 minutes). In contrast, the residues arg, lys phe, leu, trp, his, asp, and asn, all conferred low stability on the beta-galactosidase polypeptide (half-life of <3 minutes). The residue arginine (arg), when located at the amino terminus of a polypeptide, appears to generally confer the lowest stability. Thus, chimeric constructs and corresponding chimeric polypeptides employing an arg residue at the position X, described above, are generally preferred embodiments of the present invention. This is because one goal of the invention is to significantly reduce or eliminate the function of the reporter moiety in the cell.

The above described experiments establishing the relative half-lives conferred by each of the 20 possible amino terminal residues form the basis of the N-end rule. The N-end rule system components are those gene products which act to bring about the rapid proteolysis of polypeptides possessing amino-terminal residues which confer instability. The N-end rule system for proteolysis in eukaryotes appears to be a part of the general ubiquitin-dependent proteolytic system pathways possessed by apparently all eucaryotic cells. Briefly, this system involves the covalent tagging of a target polypeptide on one or more lysine residues by a ubiquitin polypeptide marker (to form a target(lys)-epsilon amino-gly(76)Ubiquitin covalent bond). Additional ubiquitin moieties may be subsequently conjugated to the target polypeptide and the resulting “ubiquitinated” target polypeptide is then subject to complete proteolytic destruction by a large (26S) multiprotein complex known as the proteasome. The enzymes which conjugate the ubiquitin moieties to the targeted protein include E2 and E3 (or ubiquitin ligase) functions. The E2 and E3 enzymes are thought to possess most of the specificity for ubiquitin dependent proteolytic processes.

A key component of the N-end rule proteolytic pathway in yeast is UBR1 (Bartel, et al. (1990) EMBO J. 9: 3179-89), a gene which encodes an E3 like function which appears to recognize polypeptides possessing susceptible amino terminal residues and thereby facilitates ubiquitination of such polypeptides (Dohmen et al. (1991) Proc. Natl. Acad. Sci. USA 88: 7351-55). Accordingly UBR1 can be used as a regulatable N-end rule component which is the effector of proteolytic degradation of the target gene polypeptide. The UBR1 gene has now been cloned from a mammalian organism (Kwon et al. (1998) Proc. Natl. Acad. Sci. USA 95: 7893-903) as well as from yeast. Thus the construction of a UBR1 mouse cell line knockout is imminent and so control of the instability of X-Reporter fusions can be further manipulated by controlling the level of UBR1 expressed.

The UBR1 gene is particularly central to the invention because it can be selectively used in conjunction with any of the above described non-methionine “X” amino-terminal destabilizing residues including: the most destabilizing—arg; strongly destabilizing residues—such as lys phe, leu, trp, his, asp, and asn; and moderately destabilizing residues—such as tyr, ile, glu, or gln. Indeed, it is an object of the present invention to provide a means, where desired, to not completely shut-off a negative selectable marker's function, but merely to attenuate it to some set degree. This can be achieved using the method of the present invention in any of a number of ways. For example, a moderately destabilizing amino-terminal residue (X=tyr, ile, glu, or gln) can be deployed on the target polypeptide reporter—resulting in a less rapid removal of the target polypeptide pool.

Other N-end rule components for use in the present invention include S. cerevisiae UBC2 (RAD6), which encodes an E2 ubiquitin conjugating function which cooperates with the UBR1-encoded N-end rule E3 to promote multiubiquitination and subsequent degradation of N-end rule substrates (Dohmen et al. (1991) Proc. Natl. Acad. Sci. USA 88: 7351-55). Thus N-end rule directed proteolysis will not occur in the absence of either UBR1 or UBC2. This allows either gene to be used as the inducible “effector of targeted proteolysis” by the method of the present invention. Indeed, a target gene polypeptide possessing an N-end rule destabilizing amino-terminal amino acid (such as arg) will be stable until expression of either the UBR1 (E3) or the UBC2 (E2) is induced from the cognate inducible promoter construct.

Both UBR1 and UBC2 can be used in conjunction with any of the above described “X” amino-terminal destabilizing residues including: the most destabilizing—arg; strongly destabilizing residues—such as lys phe, leu, trp, his, asp, and asn; and moderately destabilizing residues—such as tyr, ile, glu, or gln. Still other alternative embodiments of the N-end rule component of the present invention are components of the N-end rule system which affect only a subset of the destabilizing residues. For example, the NTA1 deamidase (Baker and Varshavsky (1995) J Biol Chem 270: 12065-74) functions to deaminate amino-terminal asn or gln residues (to form polypeptides with asp or glu amino-terminal residues respectively). Yeast strains harboring nta1 null alleles are unable to degrade N-end rule substrates that bear amino-terminal asn or gln residues. Thus, the NTA1 gene is an alternative embodiment of the N-end rule component of the present invention, but is used preferably in conjunction with a target gene polypeptide (X-target), in which X is either asn or gln. Similarly the ATE1 transferase (Balzi et al. (1990) J. Biol Chem 265: 7464-71) is an enzyme which acts to transfer the arg moiety from a tRNA˜Arg activated tRNA to amino-terminal glu or asp bearing polypeptides. The resulting arg-glu-polypeptide and arg-asp-polypeptide products are then susceptible to the E2/E3—mediated N-end rule dependent proteolytic processes described above. Thus, the ATE1 transferase is an alternative embodiment of the N-end rule component of the present invention, but its use is preferably tied to target gene polypeptides (X-target), in which X is asp, glu, asn or gln. Polypeptides bearing the latter two amino-terminal residues are first converted to polypeptides bearing one of the former two amino-terminal residues by NTA1 deamidase function described above.

From the description above, it is apparent to a skilled artisan that different cell types might possess different N-end rule components. Therefore, it might be necessary and important to genetically engineer a given cell line so that a complementation screen based on the instant invention can be successfully carried out in that given cell line. For example, many libraries or constructs generated for use in mammalian systems might be easily adapted for use in a different cell type if that cell type has the same or very similar N-end rule components and operates essentially the same as mammalian cells. However, if that cell type has dramatically different N-end rule components, it might be worthwhile to genetically modify the cell type so that available reagents can be readily used, rather than regenerate reagents for use in that particular cell line. For example, the N-end rule components may be provided as a clone so that it they can be put under the control of an inducible promoter (using standard subcloning methods well known in the art). It is also possible that other genetic engineering steps can be performed in a given cell type to make it suitable for expression of source DNA in libraries using mammalian expression vectors.

The techniques used for such genetic engineering involve stable expression of genes, which genes may potentially be heterologous to the cell type employed, and/or “knocking-out” genes, techniques which are well known in the art and can be readily appreciated by a skilled artisan.

4.5. Ubiquitin Polypeptide Sequences

A complete and detailed description of the Cub and Nub constructs which can be used in the method of the present invention have been described in U.S. Pat. Nos. 5,503,977 and 5,585,245. A background to the molecular biology of the ubiquitin proteolytic system in general, and the N-end rule system and ubiquitin sensor association assay is presumed of the skilled artisan seeking to practice the present invention. Briefly, ubiquitin (Ub) is a 76-residue, single-domain protein whose covalent coupling to other proteins yields branched Ub-protein conjugates and plays a role in a number of cellular processes, primarily through routes that involve protein degradation. Unlike the branched Ub conjugates, which are formed posttranslationally, linear Ub adducts are the translational products of natural or engineered Ub fusions. It has been shown that, in eukaryotes, newly formed Ub fusions are rapidly cleaved at the Ub-polypeptide junction by Ub-specific proteases (UBPs). In the yeast Saccharomyces cerevisiae, there are at least five species of UBP. Recent work has shown that the cleavage of a Ub fusion by UBPs requires the folded conformation of Ub, because little or no cleavage is observed with fusions whose Ub moiety was conformationally destabilized by single-residue replacements or a deletion distant from the site of cleavage by UBPs.

The present invention relies in part upon the previously described split ubiquitin protein sensor system (see U.S. Pat. Nos. 5,503,977 & 5,585,245). Briefly, it has been demonstrated that an N-terminal ubiquitin subdomain and a C-terminal ubiquitin subdomain, the latter bearing a reporter extension at its C-terminus, when coexpressed in the same cell by recombinant DNA techniques as distinct entities, have the ability to associate, reconstituting a ubiquitin molecule which is recognized, and cleaved, by ubiquitin-specific processing proteases which are present in all eukaryotic cells. This reconstituted ubiquitin molecule, which is recognized by ubiquitin-specific proteases, is referred to herein as a quasi-native ubiquitin moiety. As disclosed herein, ubiquitin-specific proteases recognize the folded conformation of ubiquitin. Remarkably, ubiquitin-specific proteases retained their cleavage activity and specificity of recognition of the ubiquitin moiety that had been reconstituted from two unlinked ubiquitin subdomains.

Ubiquitin is a 76-residue, single-domain protein comprising two subdomains which are relevant to the present invention, the N-terminal subdomain and the C-terminal subdomain. The ubiquitin protein has been studied extensively and the DNA sequence encoding ubiquitin has been published (Ozkaynak et al., EMBO J. 6: 1429 (1987)). The N-terminal subdomain (Nub), as referred to herein, is that portion of the native ubiquitin molecule which folds into the only alpha—helix of ubiquitin interacting with two beta -strands. Generally speaking, this subdomain comprises amino acid residues from about residue number 1 to about residue number 34-37.

The C-terminal subdomain of ubiquitin (Cub), as referred to herein, is that portion of the ubiquitin which is not a portion of the N-terminal subdomain defined in the preceding paragraph. Generally speaking, this subdomain comprises amino acid residues from about 35-38 to about 76. It should be recognized that by using only routine experimentation it would be possible to define with precision the minimum requirements at both ends of the N-terminal subdomain and the C-terminal subdomain which are necessary to be useful in connection with the present invention.

It is important to note that the term Nux refers, in preferred embodiments of the invention, to ubiquitin subdomain units which have been mutated so as to decrease their binding affinity, thereby making the Cub/Nub association dependent upon the binding of a second protein pair fused to the Cub and Nub subunits. Suitable forms of Nux are described below and still others are readily available to the skilled artisan by routine mutation and screening methods.

In order to study the interaction between members of a specific-binding pair, or of two polypeptides that may form such specific-binding pair, one member of the pair is fused to the N-terminal subdomain of ubiquitin and the other member of the specific-binding pair is fused to the C-terminal subdomain of ubiquitin. Since the members of the specific-binding pair (linked to subdomains of ubiquitin) have an affinity for one another, this affinity increases the “effective” (local) concentration of the N-terminal and C-terminal subdomains of ubiquitin, thereby promoting the reconstitution of a quasi-native ubiquitin moiety. For convenience, the term “quasi-native ubiquitin moiety” will be used herein to denote a moiety recognizable as a substrate by ubiquitin-specific proteases. In light of the fact that the N-terminal and C-terminal subdomains of ubiquitin associate to form a quasi-native ubiquitin moiety even in the absence of fusion of the two subdomains to individual members of a specific-binding pair, a preferred embodiment of the present invention exists in order to increase the resolving capacity of the method for studying such interactions. In this preferred embodiment, the N-terminal subdomain of ubiquitin is mutationally altered to reduce its ability to produce, through association with the C-termianl domain, a quasi-native ubiquitin moiety. It will be recognized by one of skill in the art that the binding interaction studies described herein are carried out under conditions appropriate for protein/protein interaction. Such conditions are provided in vivo (i.e., under physiological conditions inside living cells) or in vitro, when parameters such as temperature, pH and salt concentration are controlled in a manner intended to mimic physiological conditions. The present invention preferably uses the disclosed in vivo screening methods which have the advantage of being subject to a powerful negative selection method.

The mutational alteration of the N-terminal ubiquitin subdomain for use with the instant invention is preferably a point mutation. In light of the fact that it is essential that the reconstituted ubiquitin moiety must “look and feel” like native ubiquitin to a ubiquitin-specific protease, mutational alterations which would be expected to grossly affect the structure of the subdomain bearing the mutation are to be avoided. A number of ubiquitin-specific proteases have been reported, and the nucleic acid sequences encoding such proteases are also known (see e.g., Tobias et al., J. Biol. Chem. 266: 12021 (1991); Baker et al., J. Biol. Chem. 267: 23364 (1992)). It should be added that all of the at least five ubiquitin-specific proteases in the yeast S. cerevisiae require a folded conformation of ubiquitin for its recognition as a substrate. Extensive deletions within the N-terminal subdomains of ubiquitin are an example of the type of mutational alteration which would be expected to grossly affect subdomain structure and, therefore, are examples of types of mutational alterations which should be avoided.

In light of this consideration, the preferred mutational alteration within the Nub subunit is a mutation in which an amino acid substitution is effected. For example, the substitution of an amino acid having chemical properties similar to the substituted amino acid (e.g., a conservative substitution) is preferred. Specifically, the desired mild perturbation of ubiquitin subdomain interaction is achieved by substituting a chemically similar amino acid residue which differs primarily in the size of its side chain. Such a steric perturbation is expected to introduce a desired (mild) conformational destabilization of a ubiquitin subdomain. One goal is to reduce the affinity of the N-terminal and C-terminal subdomains for one another, not necessarily to eliminate this affinity.

For example, the mutational alteration may be introduced into the N-terminal subdomain of ubiquitin. More specifically, a first neutral amino acid residue may be replaced with a second neutral amino acid having a side chain which differs in size from the first neutral amino acid residue side chain to achieve the desired decrease in affinity. For example, the first neutral amino acid residue isoleucine (either residue 3 or 13 of wild-type ubiquitin) may be replaced with a neutral amino acids which has a side chain which differs in size from isoleucine such as glycine, alanine or valine.

A wide variety of fusion construct combinations can be used in the methods of this invention. One strict requirement which applies to all N- and C-terminal fusion construct combinations is that the C-terminal subdomain must bear an amino acid (e.g., peptide, polypeptide or protein) extension. This requirement is based on the fact that the detection of interaction between two proteins of interest linked to two subdomains of ubiquitin is achieved through cleavage after the C-terminal residue of the quasi-native ubiquitin moiety, with the formation of a free reporter protein (or peptide) that had previously been linked to a C-terminal subdomain of ubiquitin. Ubiquitin-specific proteases cleave a linear ubiquitin fusion between the C-terminal residue of ubiquitin and the N-terminal residue of the ubiquitin fusion partner, but they do not cleave an otherwise identical fusion whose ubiquitin moiety is conformationally perturbed. In particular, they do not recognize as a substrate a C-terminal subdomain of ubiquitin linked to a “downstream” reporter sequence, unless this C-terminal subdomain associates with an N-terminal subdomain of ubiquitin to yield a quasi-native ubiquitin moiety.

Furthermore, the characteristics of the C-terminal amino acid extension of the C-terminal ubiquitin subdomain must be such that the products of the cleaved fusion protein are distinguishable from the uncleaved fusion protein. In practice, this is generally accomplished by monitoring a physical property or activity of the C-terminal extension which is cleaved free from the C-terminal ubiquitin moiety. It is generally a property of the free C-terminal extension that is monitored as an indication that a quasi-native ubiquitin has formed, because monitoring of the quasi-native ubiquitin moiety directly is difficult in eukaryotic cells due to the presence of native ubiquitin. While unnecessary for the practice of the present invention, it would of course be appropriate to monitor directly the presence of the quasi-native ubiquitin as well, provided that this monitoring could be carried out in the absence of interference from native ubiquitin (for example, in prokaryotic cells, which naturally lack ubiquitin).

The size of the C-terminal extension which is released following cleavage of the quasi-native ubiquitin moiety within a reporter fusion by a ubiquitin-specific protease is a particularly convenient characteristic in light of the fact that it is relatively easy to monitor changes in size using, for example, electrophoretic methods. For instance, if the C-terminal reporter extension has a molecular weight of about 20 kD, the cleavage products will be distinguishable from the non-cleaved quasi-native ubiquitin moiety by virtue of the appearance of a previously absent reporter-specific 20 kD band following cleavage of the reporter fusion.

In light of the fact that the cleavage can take place, for example, in crude cell extracts or in vivo, it is generally not possible to monitor such changes in molecular weight of cleavage products by simply staining an electrophoretogram with a dye that stains proteins nonspecifically, because there are too many proteins in the mixture to analyze in this manner. One preferred method of analysis is immunoblotting. This is a conventional analytical method wherein the cleavage products are separated electrophoretically, generally in a polyacrylamide gel matrix, and subsequently transferred to a charged solid support (e.g., nitrocellulose or a charged nylon membrane). An antibody which binds to the reporter of the ubiquitin-specific protease cleavage products is then employed to detect the transferred cleavage products using routine methods for detection of the bound antibody.

Another useful method is immunoprecipitation of either a reporter-containing fusion to C-terminal subdomains of ubiquitin or the free reporter (liberated through the cleavage by ubiquitin-specific proteases upon reconstitution of a quasi-native ubiquitin moiety) with an antibody to the reporter. The proteins to be immunoprecipitated are first labeled in vivo with a radioactive amino acid such as S³⁵-methionine, using methods routine in the art. A cell extract is then prepared, and reporter-containing proteins are precipitated from the extract using an anti-reporter antibody. The immunoprecipitated proteins are fractionated by electrophoresis in a polyacrylamide gel, followed by detection of radioactive protein species by autoradiography or fluorography.

A preferred experimental design is to extend the C-terminal subdomain of ubiquitin with a peptide containing an epitope foreign to the system in which the assay is being carried out. It is also preferable to design the experiment so that the C-terminal reporter extension of the C-terminal subdomain of ubiquitin is sufficiently large, i.e., easily detectable by the electrophoretic system employed. In this preferred embodiment, the C-terminal reporter extension of the C-terminal subdomain should be viewed as a molecular weight marker. In this embodiment, the characteristics of the extension other than its molecular weight and immunological reactivity are not of particular significance. It will be recognized, therefore, that this C-terminal extension can represent an amalgam comprising virtually any amino acid sequence combination fused to an epitope for which a specifically binding antibody is available. For example, the C-terminal extension of the C-terminal ubiquitin subdomain may be a combination of the “ha” epitope fused to mouse DHFR (an antibody to the “ha” epitope is readily available).

Aside from the molecular weight of the C-terminal amino acid extension of the C-terminal ubiquitin subdomain, other characteristics can also be monitored in order to detect cleavage of a quasi-native ubiquitin moiety. For example, the enzymatic activity of some proteins can be abolished by extending their N-termini. Such a “reporter” enzyme, which, in its native form, exhibits an enzymatic activity that is abolished when the enzyme is N-terminally extended, can also serve as the C-terminal reporter linked to the C-terminal ubiquitin subdomain.

In this detection scheme, when the reporter is present as a fusion to the C-terminal ubiquitin subdomain, the reporter protein is inactive. However, if the C-terminal ubiquitin subdomain and the N-terminal ubiquitin subdomain associate to reconstitute a quasi-native ubiquitin moiety in the presence of a ubiquitin-specific protease, the reporter protein will be released, with the concomitant restoration of its enzymatic activity.

In preferred embodiments, the reporter protein is a eukaryotic negative selectable marker (NSM) which has been engineered to be processed and released as an N-end rule-labile X-NSM fusion following UBP proteolytic cleavage. The negative selectable markers (NSMs) for use in the invention are described elsewhere herein. The advantage of using an X-NSM fusion is that interaction of the specific binding pair can be directly selected for (as opposed to screened for) by virtue of the fact that only cells in which X-NSM has been released will survive negative selection.

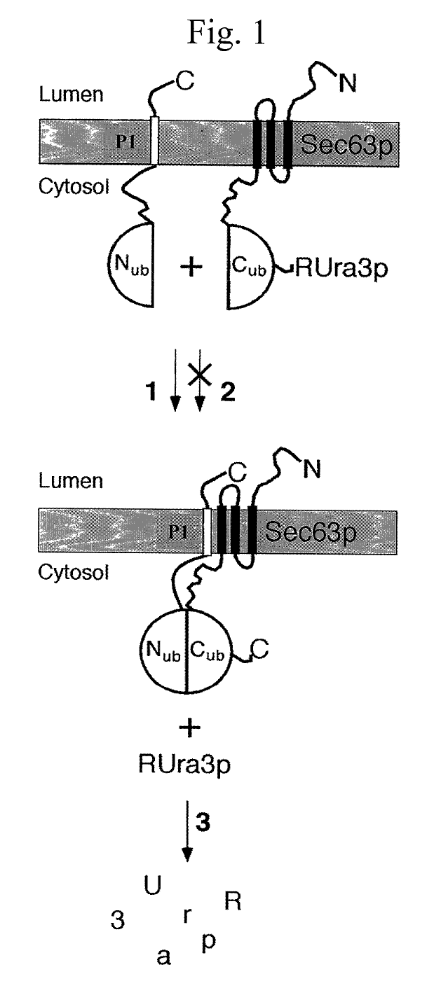

As shown in FIG. 1, the target gene reporter (negative selectable marker) may be fused downstream of a codon which encodes an N-end rule susceptible residue (X, as described above) and this residue, in term, must be fused in-frame to the carboxy-terminus of a ubiquitin coding sequence (generally the carboxy-terminus of a C-terminal ubiquitin subdomain (Cub) which corresponds to gly76 of intact ubiquitin). The reason for constructing this extensive chimeric gene construct is to take advantage of the ability of constitutive ubiquitin proteases to cleave any peptide bond which is carboxy-terminal to gly76 of an intact ubiquitin unit.

The summary description in the preceding paragraph does not discuss certain important experimental considerations. For example, for two interacting proteins, P1 (fused to Nub) and P2 (fused to Cub), the following additional considerations are included within the scope of the invention. In light of its role as an affinity component, it will be recognized that P1 can be fused to the N-terminus or the C-terminus of the N-terminal ubiquitin subdomain. Similarly, P2 can be fused to the N-terminus or the C-terminus of the C-terminal ubiquitin subdomain. If P2 is fused to the C-terminus of the C-terminal ubiquitin subdomain, it will be removed by cleavage by the ubiquitin-specific protease, providing that the ubiquitin subdomains associate to form a quasi-native ubiquitin moiety. Consistent with the summary description in the preceding paragraph, if the P2 moiety is fused to the C-terminus of the C-terminal ubiquitin subdomain, it may also be used as a reporter for detecting reconstitution of a quasi-native ubiquitin moiety. Furthermore, the position of P2 within the C-terminal reporter-containing region of the fusion is not a critical consideration.

4.6. Libraries and Screening methods for the Screening of Novel Interaction Partners for a Given Polypeptide

The present invention provides methods to determine whether two proteins bind to each other. When trying to use such methods for the identification of a previously unknown binding partner for a given polypeptide, one preferably will use a library of polypeptides and screen for members of such library that are capable of interacting with the given polypeptide. This is, for example, carried out by constructing a cDNA or genomic library, cloning this library into a vector comprising the Nux-construct, and expressing the library of vectors so created in a host cell expressing a fusion protein comprising the given polypeptide and the Cub-X-RM polypeptide. This section shall outline methods to generate libaries for use in such methods, and how these libraries may be employed to characterize a novel polypeptide interacting with the given polypeptide.

Library Construction

At least two important aspects of library construction need to be considered. One is the source of DNA, the other is the choice of vector suitable for the library.

Many different types of source DNA can be used for library construction. One of the most commonly used source is complementary DNA (cDNA), which is normally obtained by reverse transcription of mRNA isolated from cell lines or tissues, followed by second strand synthesis to complete the synthesis of double-stranded cDNA. The synthesis of cDNA is common knowledge and there are numerous commercially available kits and laboratory manuals covering this subject, and therefore it will not be discussed further.

Genomic DNA (gDNA) is another major source of DNA, although it is less common for construction of an expression library, largely due to the presence of introns and other non-coding regions. The isolation of genomic DNA and size fractionation into suitable pieces for library construction is also well-known in the art.

Other DNA sources can also be used. For example, random or semi-random polynucleotide sequences can be used as source DNA for library construction. This is a particularly powerful method when small stretches of these random fragments are incorporated into a known coding sequence to screen for optimal sequences for certain activity, i.e. binding between two proteins or enzymatic activity.

Many vectors are suitable for library construction. Generally, the chosen vector shall have at least one cloning site for insertion of source DNA. The most commonly used cloning sites are restriction enzyme sites, preferably those restriction enzymes that rarely cut inside coding sequences, such as NotI, SalI. However, other sites can also be used. For example, loxP sites can be used instead of or in addition to restriction enzyme sites. Such sites flanking the cloned source DNA can be recognized by Cre recombinase and readily excised in a controlled manner since Cre recombinase can be conditionally provided by induced expression. Many other similar recombination-based systems are also commercially available, such as the Gateway system (Life Technology, Inc.) that is described in U.S. Pat. No. 5,888,732, the content of which is incorporated by reference herein.

The vector shall also be suitable for expression of the cloned source DNA, either in vitro or in vivo. At the minimum, it shall have a promoter for transcription of the DNA in its intended host. The host can be a mammalian cell, an insect cell, or a plant cell, or any other cell as specified in other sections of this specification. The vector shall also have the ability to maintain itself in the host cell, at least during the pendency of the experiment. That can be achieved by self replication or integration into the host genome. Some vector may also contain selectable markers to facilitate easy identification of cells that have accepted/maintained the vector, and thus the source DNA.

Numerous vectors fit into the definition as outlined above. For example, but without limitation, U.S. Pat. Nos. 5,521,093, 5,538,863, 5,637,504, 5,866,404, and 6,221,588 provide ample examples of yeast vectors suitable for expression of heterologous genes, the contents of which are all incorporated herein in their entirety.

Furthermore, a large number of vectors developed for expression in mammalian cells fulfill the requirements as outlined above. U.S. Pat. No. 6,255,071 has detailed description of a variety of viral vectors suitable for mammalian expression screen, which is incorporated herein by reference in its entirety. Specifically, U.S. Pat. No. 6,255,071 relates to methods and compositions for improved mammalian complementation screening, functional inactivation of specific essential or non-essential mammalian genes, and identification of mammalian genes which are modulated in response to specific stimuli. In particular, it discloses replication-deficient retroviral vectors, libraries comprising such vectors, retroviral particles produced by such vectors in conjunction with retroviral packaging cell lines, integrated provirus sequences derived from the retroviral particles and circularized provirus sequences which have been excised from the integrated provirus sequences. It further discloses novel retroviral packaging cell lines for use for those viral vectors. Exemplary vectors disclosed by the patent are:

1) A retroviral vector containing a polycistronic message cassette, a proviral excision element for excising retroviral provirus from the genome of a recipient cell and a proviral recovery element for recovering excised provirus from a complex mixture of nucleic acid, a 5′ retroviral long terminal repeat (5′ LTR), a 3′ retroviral long terminal repeat (3′ LTR), a packaging signal, a bacterial origin of replication, and a selectable marker. The retroviral vector may also contain a polycistronic message cassette which makes possible a selection scheme that directly links expression of a selectable marker to transcription of a cDNA or genomic DNA (gDNA) sequence. Such a polycistronic message cassette can comprise, in one embodiment, from 5′ to 3′, the following elements: a nucleotide polylinker, an internal ribosome entry site and a mammalian selectable marker. The polycistronic cassette is situated within the retroviral vector between the 5′ LTR and the 3′ LTR at a position such that transcription from the 5′ LTR promoter transcribes the polycistronic message cassette. The transcription of the polycistronic message cassette may also be driven by an internal cytomegalovirus (CMV) promoter or an inducible promoter, which may be preferable depending on the screenings. The polycistronic message cassette can further comprise a cDNA or genomic DNA (gDNA) sequence operatively associated within the polylinker.

Internal ribosome entry site sequences are well known to those of skill in the art and can comprise, for example, internal ribosome entry sites derived from foot and mouth disease virus (FDV), encephalomyocarditis virus, poliovirus and RDV (Scheper, 1994, Biochemic 76: 801-809; Meyer, 1995, J. Virol. 69: 2819-2824; Jang, 1988, J. Virol. 62: 2636-2643; Haller, 1992, J. Virol. 66: 5075-5086).

Any mammalian selectable marker can be utilized as the polycistronic message cassette mammalian selectable marker. Such mammalian selectable markers are well known to those of skill in the art and can include, but are not limited to, kanamycin/G418, hygromycinB or mycophenolic acid resistance markers. Other examples are provided elsewhere herein.

The retroviral vectors' proviral excision element allows for excision of retroviral provirus (see below) from the genome of a recipient cell. The element comprises a nucleotide sequence which is specifically recognized by a recombinase enzyme. The recombinase enzyme cleaves nucleic acid at its site of recognition in such a manner that excision via recombinase action leads to circularization of the excised nucleic acid molecules.

In a preferred embodiment, the recombinase recognition site is located within the 3′ LTR at a position which is duplicated upon integration of the provirus. This results in a provirus that is flanked by recombinase sites.

In another preferred embodiment, the proviral excision element comprises a loxP recombination site, which is cleavable by a Cre recombinase enzyme. Contacting Cre recombinase to an integrated provirus derived from the retroviral vector results in excision of the provirus nucleic acid. In the alternative, a mutant lox P recombination site may be used (e.g., lox P511 (Hoess et al., 1986, Nucleic Acids Research 14:2287-2300)) that can only recombine with an identical mutant site.

In yet another preferred embodiment, an FRT recombination site, which is cleavable by a FLP recombinase enzyme, is utilized in conjunction with FLP recombinase enzyme, as described above for the loxP/Cre embodiment. In yet an alternative embodiment, a rare-cutting restriction enzyme (e.g., Not I) may be used in place of the recombinase site. The recovered DNA would be digested with Not I and then recircularized with ligase. In this embodiment, the Not I site is included in the vector next to loxP. In still another embodiment, an r recombinase site and r recombinase from Zygosaccharomyces rouxii can be utilized, as described above, for the loxP/Cre embodiment.

In the complementation screening system of the invention, described below, such excision systems can also serve to discriminate revertants from virus-dependent rescue events.

The retroviral vectors' proviral recovery element allows for recovery of excised provirus from a complex mixture of nucleic acid, thus allowing for the selective recovery and excision of provirus from a recipient cell genome. The proviral recovery element comprises a nucleic acid sequence which corresponds to the nucleic acid portion of a high affinity binding nucleic acid/protein pair.

The nucleic acid can include, but is not limited to, a nucleic acid which binds with high affinity to a lac repressor, tet repressor or lambda repressor protein. For example, in one embodiment, the proviral recovery element comprises a lac operator nucleic acid sequence, which binds to a lac repressor peptide sequence. Such a proviral recovery element can be affinity-purified using lac repressor bound to a matrix (e.g., magnetic beads or sepharose). An excised provirus derived from the retroviral vectors of the invention also contains the retroviral recovery element and can be affinity purified.

The 5′ LTR comprises a promoter, including but not limited to an LTR promoter, an R region, a U5 region and a primer binding site, in that order. Nucleotide sequences of these LTR elements are well known to those of skill in the art.

The 3′ LTR comprises a U3 region which comprises the proviral excision element, a promoter, an R region and a polyadenylation signal. Nucleotide sequences of such elements are well known to those of skill in the art.

The bacterial origin of replication (Ori) utilized is preferably one which does not adversely affect viral production or gene expression in infected cells. As such, it is preferable that the bacterial Ori is a non-pUC bacterial Ori relative (e.g., pUC, colEI, pSC101, p15A and the like). Further, it is preferable that the bacterial Ori exhibit less than 90% overall nucleotide similarity to the pUC bacterial Ori. In a preferred embodiment, the bacterial origin of replication is a RK2 OriV or f1 phage Ori.

Any bacterial selectable marker can be utilized. Bacterial selectable markers are well known to those of skill in the art and can include, but are not limited to, kanamycin/G418, zeocin, actinomycin, ampicillin, gentamycin, tetracycline, chloramphenicol or penicillin resistance markers.

The retroviral vectors can further comprise a lethal stuffer fragment which can be utilized to select for vectors containing cDNA or gDNA inserts during, for example, construction of libraries comprising the retroviral vectors of the invention. Lethal stuffer fragments are well known to those of skill in the art (see, e.g., Bernord et al., 1994, Gene 148:71-74, which is incorporated herein by reference in its entirety). A lethal stuffer fragment contains a gene sequence whose expression conditionally inhibits cellular growth.

In one embodiment, the stuffer fragment is present in the retroviral vectors of the invention within the polycistronic message cassette polylinker such that insertion of a cDNA or gDNA sequence into the polylinker replaces the stuffer fragment. Alternatively, the polycistronic message cassette polylinker is located within the lethal stuffer fragment coding sequence such that, upon insertion of a cDNA or gDNA sequence into the polylinker, the lethal stuffer fragment coding region is disrupted. Each of these embodiments can be utilized to counter select retroviral vectors not containing polylinker insertions.

The retroviral vectors can further comprise a single-stranded replication origin, preferably an f1 single-stranded replication origin. The single-stranded replication origin allows for the production of normalized single-stranded retroviral libraries derived from the retroviral vectors of the invention. A normalized library is one constructed in a manner that increases the relative frequency of occurrence of rare clones while decreasing simultaneously the relative frequency of the occurrence of abundant clones. For teaching regarding the production of normalized libraries, see, e.g., Soares et al. (Soares, M. B. et al., 1994, Proc. Natl. Acad. Sci. USA 91:9228-9232, which is incorporated herein by reference in its entirety). Alternative normalization procedures based upon biotinylated nucleotides may also be utilized.

2) A mammalian episomal vector, termed pEHRE vector, which makes possible, stable, efficient, high-level episomal expression within a wide spectrum of mammalian cells. Such vectors can also, for example, be utilized as part of the complementation screening methods of the invention.

Such pEHRE expression vectors comprise a replication cassette, an expression cassette and minimal cis-acting elements necessary for replication and stable episomal maintenance.

The pEHRE vectors can further contain at least one bacterial origin of replication and/or recombination sites. The recombination sites preferably flank the replication cassette, and can include, but are not limited to, any of the recombination sites described above.

Any bacterial origin of replication (Ori) which does not adversely affect the expression of pEHRE sequences can be utilized. For example, the bacterial Ori can be a pUC bacterial Ori relative (e.g., pUC, colEI, pSC101, p15A and the like). The bacterial origin of replication can also, for example, be a RK2 OriV or f1 phage Ori. The pEHRE vectors can further comprise a single stranded replication origin, preferably an f1 single-stranded replication origin. The single-stranded replication origin allows for the production of normalized single-stranded libraries derived from the pEHRE vectors of the invention.

In instances wherein an f1 origin of replication is utilized, the pEHRE vectors can additionally comprise a nucleic acid sequence which corresponds to the nucleic acid portion of a high affinity binding nucleic acid/protein pair. Such nucleic acid/protein pairs can be those as described above, the nucleic acid portion of which can include, but is not limited to, a lacO site. The nucleic acid can include, but is not limited to, a nucleic acid which binds with high affinity to a lac repressor, tet repressor or lambda repressor protein. For example, in one embodiment, the proviral recovery element comprises a lac operator nucleic acid sequence, which binds to a lac repressor peptide sequence. Such a proviral recovery element can be affinity-purified using lac repressor bound to a matrix (e.g., magnetic beads or sepharose). An excised provirus derived from the retroviral vectors of the invention also contains the retroviral recovery element and can be affinity purified.

A pEHRE vector replication cassette comprises nucleic acid sequences which encode papillomaviruses (PV) E1 and E2 proteins, wherein such nucleic acid sequences are operatively attached to and transcribed by, a constitutive transcriptional regulatory sequence. Representative E1 and E2 amino acid sequences are well known to those of skill in the art. See, e.g., sequences publicly available in databases such as Genbank. The E1 and E2 coding sequences can, first, include any nucleotide sequences which encode endogenous PV, including but not limited to bovine papillomavirus (BPV), such as BPV-1 E1 or E2 gene products.

As used herein, the term “E1” also refers to any protein which is capable of functioning in PV in the same manner as the endogenous E1 protein, i.e., is capable of complementing an E1 mutation. Taking BPV as an example, an E1 protein, as described herein, is one capable of complementing a BPV E1 mutation. Likewise, the term “E2”, as used herein, refers to any protein which is capable of functioning in PV in the same manner as the endogenous E2 protein, i.e., is capable of complementing a E2 mutation. Taking BPV as an example, an E2 protein, as described herein, is one capable of complementing a BPV E2 mutation.

The replication cassette constitutive transcriptional regulatory sequence can include, but is not limited to, any polII promoter, such as an SV40, CMV or PGK promoter, nucleotide sequences of which are well known to those of skill in the art.

E1 and E2 coding sequences can be operatively attached to, and transcribed by, separate transcriptional regulatory sequences. In one embodiment, at least one of the E1 or E2 coding sequences can be transcribed along with a selectable marker as a polycistronic message. Such a polycistronic message construction makes possible a selection scheme which directly links expression of a selectable marker, preferably a mammalian selectable marker, to transcription of a sequence necessary for episomal maintenance and replication. For example, the portion of a replication cassette encoding such a polycistronic message could comprise, from 5′ to 3′: a constitutive transcriptional regulatory sequence, an E2 (or E1) coding sequence, an internal ribosome entry site (IRES), and a selectable marker.

In another embodiment, both E1 and E2 coding sequences can be transcribed as a polycistronic message. That is, both E1 and E2 coding sequences, separated by an internal ribosome entry site, can be transcribed by a single transcriptional regulatory sequence.

In yet another embodiment, E1, E2 and selectable marker sequences can be transcribed as a polycistronic message. For example, the replication cassette could comprise, from 5′ to 3′: a constitutive transcriptional regulatory sequence, an E2 (or E1) coding sequence, an IRES, an E1 (or E2) coding sequence, an IRES and a selectable marker.

In instances wherein the E1 and E2 coding sequences are transcribed as part of a polycistronic message, it is preferred that the order, from 5′ to 3′, be E2 then E1. This is to ensure against possible rare, undesirable RNA splicing events.

The pEHRE vector expression cassette is designed to yield high level expression of a cDNA or genomic DNA (gDNA) sequence. Such a pEHRE vector expression cassette comprises, from 5′ to 3′, a transcriptional regulatory sequence, a nucleotide polylinker, an internal ribosome entry site, a mammalian selectable marker and, preferably, either a poly-A site or a transcriptional termination sequence, depending upon the transcriptional regulatory sequence utilized (see below). A cDNA or gDNA sequence can be expressed via operative association within the polylinker. A pEHRE expression vector can contain a single or multiple expression cassettes, such that greater than one cDNA or gDNA sequence can be expressed from the same pEHRE expression vector.

The pEHRE vector expression cassette transcriptional regulatory sequence can be either constitutive or inducible, and can be derived from cellular or viral sources. For example, such transcriptional regulatory sequences can include, but are not limited to, a retroviral long terminal repeat (LTR), cytomegalovirus (CMV), Va-1 RNA or U6 snRNA promoter sequence, nucleotide sequences of which are well known to those of skill in the art. Depending upon the transcriptional regulatory sequence chosen, the expression cassette can contain either a poly-A site (pA) or a transcriptional termination sequence. One of skill in the art will readily be able to choose, without undue experimentation, the appropriate sequence to be used with any given transcriptional regulatory sequence. In general, for example, polII-type transcriptional regulatory sequences can be coupled with pA sites, and polII-type transcriptional regulatory sequences can be coupled with transcriptional termination sequences.

Expression from the transcriptional regulatory sequence yields a polycistronic message comprising the cDNA or gDNA sequence of interest, IRES and mammalian selectable marker. Such a polycistronic message approach allows a selection scheme which ensure that the cDNA or GDNA of interest has been expressed.

The pEHRE vectors further comprise cis-acting elements which function in replication and stable episomal maintenance. Such sequences include: a PV minimal origin of replication (MO) and a PV minichromosomal maintenance element (MME). Representative MO and MME sequences are well known to those of skill in the art. See, e.g., Piirson, M. et al., 1996, EMBO J. 15:1-11, which is incorporated herein by reference in its entirety.

As used herein, the term “MO” refers to any nucleotide sequence capable of functioning in PV in the same manner as endogenous MO, i.e., is capable of complementing an MO mutation. Taking BPV as an example, an MO sequence, as described herein, would be one capable of complementing or replacing a BPV MO mutation. Likewise, the term “MME”, as used herein, refers to any nucleotide sequence capable of functioning in PV in the same manner as endogenous MME, i.e., is capable of complementing a MME mutation. For example, a MME sequence can be one containing multiple E2 binding sites. Taking BPV as an example, a MME sequence, as described herein, would be one capable of complementing or replacing a BPV MME mutation.

The pEHRE IRES and mammalian and bacterial selectable markers can be, for example, as those described above.

The pEHRE expression vectors of the invention can be utilized for the production, including large scale production, of recombinant proteins. The vectors' desirable features, in fact, make them especially amenable to large scale production. Specifically, current methods of producing recombinant proteins in mammalian cells involve transfection of cells (e.g., CHO, NS/0 cells) and subsequent amplification of the transfected sequence using drugs (e.g., methotrexate or inhibitors of glutamine synthetase). Such approaches suffer for a variety of reasons, including the fact that amplicons are subject to statistical variation depending on their genomic integration loci, and from the fact that the amplicons are unstable in the absence of continued selection (which is impractical at production scale). The pEHRE vectors, it should be pointed out, achieve such levels equal or higher than these naturally, that is, in the absence of outside selection.

The pEHRE vectors give consistently high episomal expression, making them genomic integration-independent. Further, the episomal pEHRE vectors are retained as stable nuclear plasmids even in the absence of selective pressure.

Further, pEHRE vectors can be utilized which employ an additional level of such internal, or self, selection (that is, selection which does not depend on the addition of outside selective pressures such as, e.g., drugs). For example, pEHRE vectors can be utilized which complement a defect the specific producer cell line being utilized for expression. By way of example, and not by way of limitation, such pEHRE selection elements can complement an auxotrophic mutation or can bypass a growth factor requirement (e.g., proline or insulin, respectively) from the cell media. Preferably, the coding sequence of the marker is transcribed as part of a polycistronic message along with the coding sequence of the proteins being recombinantly expressed. For example, such an expression/selection cassette can comprise, from 5′ to 3′: a transcriptional regulatory sequence, recombinant protein coding sequence, IRES, selection marker, poly-A site.

The episomal pEHRE vectors can further be utilized, for example, in the delivery of large nucleic acid segments, e.g., chromosomal segments. In one such embodiment, pEHRE vectors can be utilized in connection with bacterial artificial chromosome (BAC) or yeast artificial chromosome (YAC) sequences to allow delivery of large genomic segments (e.g., segments ranging from tens of kilobases to megabases in length). For clarity, the discussion that follows describes vectors that utilize BAC sequences, but it is to be understood that vectors of the sort described here can, alternatively, utilize YAC sequences.

In one embodiment, pEHRE vectors can be combined with existing BAC clones to generate pEHRE/BAC hybrid constructs, comprising BACs into which pEHRE vector sequences have been inserted. Such pEHRE/BAC hybrids represent BACs that can replicate in a wide variety of mammalian, including human cells.

In general, pEHRE vectors which can be utilized to donate elements to BACs comprise a pEHRE replication cassette, MO and MME sequences, and a bacterial selectable marker, all flanked by BAC recombination sequences. The remainder of the vector can further comprise at least one bacterial origin of replication and a second bacterial selectable marker.

BAC recombination sequences caN include any nucleotide sequence which can be cleaved and then used to recombine with BAC elements so as to incorporate the necessary pEHRE sequences described above. Any recombination site for which a compatible recombination site exists, or is engineered to exist, in the recipient BAC can be used. For example, such BAC recombination elements can include, but are not limited to, loxP, mutant loxP or frt sites as described above.

Alternatively, CosN sites, whose nucleotide sequences are well known to those of skill in the art, can be utilized. Rather than a recombinase enzyme, such CosN sites are cleaved by lambda terminase enzyme. (For general BAC teaching, including CosN teaching, see, e.g., Shizuya, H. et al., 1992, Proc. Natl. Acad. Sci. USA 89:8794-8797; and Kim, U.-J. et al., 1996, Genomics 34:213-218, which are incorporated herein by reference in their entirety.)

In order to recombine pEHRE and BAC sequences, pEHRE vectors and BAC (containing a recombination site compatible with the chosen pEHRE vector) are treated together with the appropriate recombinase or terminase enzyme. When the CosN/terminase system is used, a subsequent ligation step is included.

The treatment will result in a low level of concatamerization. Concatamers representing the desired pEHRE/BAC hybrids can be selected for based upon their resistance to both the BAC selectable marker (usually chloramphenicol) and the pEHRE vector selectable marker within the pEHRE region meant to be donated. It is, therefore, desirable that the BAC and pEHRE selectable markers be different. In a preferred embodiment, the resulting constructs are further tested to ensure that the second pEHRE bacterial selectable marker is no longer present. Plasmids which have recombined the desired BAC and pEHRE elements, will be able to replicate in E. coli, as well as a wide range of mammalian cells, including human cells.

The vector termed a pBPV-BacDonor vector, represents one embodiment of a pEHRE vector designed to donate essential pEHRE sequences to recipient BAC clones. The vector's recombination elements are depicted as containing loxP and/or CosN sites. The bacterial marker to be incorporated into the pEHRE/BAC hybrid is depicted as tetracycline or kanamycin. Finally, the vector contains a pUC bacterial origin (Ori) of replication, an f1 Ori and a second bacterial selectable marker, ampicillin.

In an alternative embodiment, pEHRE/BAC cloning vectors can be produced and utilized. Such vectors contain the pEHRE replication cassette, MO and MME sequences as described above, the nucleotide sequences necessary for BAC maintenance in E. coli (such sequences are well known to those of skill in the art; see, e.g., Shizuya and Kim, above), and a polylinker site.

The vector termed pBPV-BlueBAC, represents one embodiment of such a pEHRE/BAC cloning vector. In this vector, the E1 and E2 coding sequences are BPV sequences, and are in operative association with individual SV40 promoters. E1 is transcribed as part of a polycistronic message along with the selectable marker, hygro. In this embodiment, the replication cassette further comprises an SV40 pA site downstream of the IRES-marker. Further, the MO and MME sequences are BPV-derived (in the figure, both of these sequences are illustrated as “BPV origin”). The cloning site comprises a polylinker embedded within the alpha complementation fragment of lacZ, which allows blue/white selection of recombinants. T7 and SP6 promoters flank the lacZ sequence, and the vector additionally contains cosN and loxP sites for linearization. The remainder of the elements depicted are present for BAC maintenance in E. coli.

3) A genetic suppressor element (GSE)-producing replication-deficient retroviral vectors. Such vectors are designed to facilitate the expression of antisense GSE single-stranded nucleic acid sequences in mammalian cells, and can, for example, be utilized in conjunction with the antisense-based functional gene inactivation methods of the invention.

The GSE-producing retroviral vectors can comprise a replication-deficient retroviral genome containing a proviral excision element, a proviral recovery element and a genetic suppressor element (GSE) cassette.

The GSE-producing retroviral vectors can further comprise, (a) a 5′ LTR; (b) a 3′ LTR; (c) a bacterial Ori; (d) a mammalian selectable marker; (e) a bacterial selectable marker; and (f) a packaging signal.

The proviral recovery element, GSE cassette, bacterial Ori, mammalian selectable marker and bacterial selectable marker are located between the 5′LTR and the 3′ LTR. The proviral excision element is located within the 3′ LTR. The proviral excision element can also flank the functional cassette without being present in the 3′ LTR.

The 5′ LTR, 3′ LTR, proviral excision element, bacterial selectable marker, mammalian selectable marker and proviral recovery element are as described above.

Each of the GSE cassette embodiments described below can further comprise a sense or antisense cDNA or gDNA fragment or full length sequence operatively associated within the polylinker.

The GSE cassette can, for example, comprise, from 5′ to 3′: (a) a transcriptional regulatory sequence; (b) a polylinker; and (c) polyadenylation signal. In one embodiment, the GSE cassette polyadenylation signal is located within the 3′ retroviral long terminal repeat.

Alternatively, the GSE cassette can comprise, from 5′ to 3′: (a) a transcriptional regulatory sequence; (b) a polylinker; (c) a cis-acting ribozyme sequence; (d) an internal ribosome entry site; (e) the mammalian selectable marker; and (f) a polyadenylation signal.

In a further alternative, a sense GSE can be constructed, in which case the GSE cassette can further comprise a polylinker containing a Kozak consensus methionine in front of the sense-orientation fragments to create a “domain library” for domain and fragment expression.

In such an embodiment, transcription from the transcriptional regulatory sequence produces a bifunctional transcript. The first half (i.e., the portion upstream of the ribozyme sequence) is likely to remain nuclear and represents the GSE. The portion downstream of the ribozyme sequence (i.e., the portion containing the selectable marker) is transported to the cytoplasm and translated. Such a bicistronic configuration, therefore, directly links selection for the selectable marker to expression of the GSE.

In another alternative, the GSE cassette can comprise, from 5′ to 3′: (a) an RNA polymerase III transcriptional regulatory sequence; (b) a polylinker; (c) a transcriptional termination sequence. In a particular embodiment, the transcriptional regulatory sequence and transcriptional termination sequence are adenovirus Ad2 VA RNAI transcriptional regulatory and termination sequences.

(4) A genetic suppressor element (GSE)-producing pEHRE vectors. Such vectors are designed to facilitate the expression of antisense GSE single-stranded nucleic acid sequences in mammalian cells, and can, for example, be utilized in conjunction with the antisense-based functional gene inactivation methods of the invention.

The GSE-producing pEHRE vectors of the invention can comprise a replication cassette, a genetic suppressor element (GSE) cassette and minimal cis-acting elements necessary for replication and stable episomal maintenance.

The GSE-producing pEHRE vectors can further comprise at least one bacterial origin of replication and at least one bacterial selectable marker.

The replication cassette, minimal cis-acting elements, bacterial origin of replication and bacterial selectable marker are as described above.

Each of the GSE cassette embodiments described below can further comprise a sense or antisense cDNA or GDNA fragment or full length sequence operatively associated within the polylinker.

The GSE cassette can, for example, comprise, from 5′ to 3′: (a) a transcriptional regulatory sequence; (b) a polylinker; and (c) polyadenylation signal. The GSE transcriptional regulatory sequence can be a constitutive or inducible one, and can represent, for example, retroviral long terminal repeat (LTR), cytomegalovirus (CMV), Va-1 RNA or U6 snRNA promoter sequence, nucleotide sequences of which are well known to those of skill in the art.

A pEHRE GSE vector could, for example be constructed in such a way that the E1 and E2 coding sequences are BPV sequences, and are in operative association with individual SV40 promoters. E1 is transcribed as part of a polycistronic message along with the selectable marker, hygro. In this embodiment, the replication cassette further comprises an SV40 pA site downstream of the IRES-marker. Further, the MO and MME sequences are BPV-derived. The vector's GSE cassette comprises a CMV promoter operatively associated with a sequence to be expressed as a GSE, which, in turn, is operatively attached to a bgH poly-A site. Finally, the vector contains a pUC bacterial origin (Ori) of replication, an f1 Ori and an ampicillin bacterial selectable marker.

Alternatively, the GSE cassette can comprise, from 5′ to 3′: (a) a transcriptional regulatory sequence; (b) a polylinker; (c) a cis-acting ribozyme sequence; (d) an internal ribosome entry site; (e) the mammalian selectable marker; and (f) a polyadenylation signal.

In another alternative, a sense GSE can be constructed, in which case the GSE cassette can further comprise a polylinker containing a Kozak consensus methionine in front of the sense-orientation fragments to create a “domain library” for domain and fragment expression.

In such an embodiment, transcription from the transcriptional regulatory sequence produces a bifunctional transcript. The first half (i.e., the portion upstream of the ribozyme sequence) is likely to remain nuclear and represents the GSE. The portion downstream of the ribozyme sequence (i.e., the portion containing the selectable marker) is transported to the cytoplasm and translated. Such a bicistronic configuration, therefore, directly links selection for the selectable marker to expression of the GSE.

In another alternative, the GSE cassette can comprise, from 5′ to 3′: (a) an RNA polymerase III transcriptional regulatory sequence; (b) a polylinker; (c) a transcriptional termination sequence.

In a particular embodiment, the transcriptional regulatory sequence and transcriptional termination sequence are adenovirus Ad2 VA RNA transcriptional regulatory and termination sequences.

(5) A vector useful for the display of constrained and unconstrained random peptide sequences. Such vectors are designed to facilitate the selection and identification of random peptide sequences that bind to a protein of interest.

The retroviral and pEHRE vectors displaying random peptide sequences of the present invention can comprise, (a) a splice donor site or a LoxP site (e.g., LoxP511 site); (b) a bacterial promoter (e.g., pTac) and a shine-delgarno sequence; (c) a pel B secretion signal for targeting fusion peptides to the periplasm; (d) a splice-acceptor site or another LoxP511 site (Lox P511 sites will recombine with each other, but not with the LoxP site in the 3′ LTR); (e) a peptide display cassette or vehicle; (f) an amber stop codon; (g) the M13 bacteriophage gene 111 protein C-terminus (amino acids 198-406); and optionally the vector may also comprise a flexible polyglycine linker.

A peptide display cassette or vehicle consists of a vector protein, either natural or synthetic into which a polylinker has been inserted into one flexible loop of the natural or synthetic protein. A library of random oligonucleotides encoding random peptides may be inserted into the polylinker, so that the peptides are expressed on the cell surface.

The display vehicle of the vector may be, but is not limited to, thioredoxin for intracellular peptide display in mammalian cells (Colas et al., 1996, Nature 380:548-550) or may be a minibody (Tramonteno, 1994, J. Mol. Recognit. 7:9-24) for the display of peptides on the mammalian cell surface. Each of these would contain a polylinker for the insertion of a library of random oligonucleotides encoding random peptides at the positions specified above. In an alternative embodiment, the display vehicle may be extracellular, in this case the minibody could be preceded by a secretion signal and followed by a membrane anchor, such as the one encoded by the last 37 amino acids of DAF-1 (Rice et al., 1992, Proc. Natl. Acad. Sci. 89:5467-5471). This could be flanked by recombinase sites (e.g., FRT sites) to allow the production of secreted proteins following passage of the library through a recombinase expressing host.

In one embodiment of the present invention, these cassettes would reside at the position normally occupied by the cDNA in the sense-expression vectors described above. In an amber suppressor strain of bacteria and in the presence of helper phage, these vectors would produce a relatively conventional phage display library which could be used exactly as has been previously described for conventional phage display vectors. Recovered phage that display affinity for the selected target would be used to infect bacterial hosts of the appropriate genotype (i.e., expressing the desired recombinases depending upon the cassettes that must be removed for a particular application). For example for an intracellular peptide display, any bacterial host would be appropriate (provided that splice sites are used to remove pelB in the mammalian host). For a secreted display, the minibody vector would be passed through bacterial cells that catalyze the removal of the DAF anchor sequence. Plasmids prepared from these bacterial hosts are used to produce virus for assay of specific phenotypes in mammalian cells.

The advantage of these vectors over conventional approaches is their flexibility. The ability to functionally test the peptide sequence in mammalian cells without additional cloning or sequencing steps makes possible the use of much cruder binding targets (e.g., whole fixed cells) for phage display. This is made possible by the ability to do a rapid functional selection on the enriched pool of bound phages by conversion to retroviruses that can infect mammalian cells.(6) A replication-deficient retroviral gene trapping vector. Such gene trapping vectors contain reporter sequences which, when integrated into an expressed gene, “tag” the expressed gene, allowing for the monitoring of the gene's expression, for example, in response to a stimulus of interest. The gene trapping vectors of the invention can be used, for example, in conjunction with the gene trapping-based methods of the invention for the identification of mammalian genes which are modulated in response to specific stimuli.

The replication-deficient retroviral gene trapping vectors of the invention can comprise: (a) a 5′ LTR; (b) a promoterless 3′ LTR (a SIN LTR); (c) a bacterial Ori; (d) a bacterial selectable marker; (e) a selective nucleic acid recovery element for recovering nucleic acid containing a nucleic acid sequence from a complex mixture of nucleic acid; (f) a polylinker; (g) a mammalian selectable marker; and (h) a gene trapping cassette. In addition, those elements necessary to produce a high titer virus are required. Such elements are well known to those of skill in the art and contain, for example, a packaging signal.

The bacterial Ori, bacterial selectable marker, selective nucleic acid recovery element, polylinker, and mammalian selectable marker are located between the 5′ LTR and the 3′ LTR. The bacterial selectable marker and the bacterial Ori are located in close operative association in order to facilitate nucleic acid recovery, as described below. The gene trapping cassette element is located within the 3′ LTR.

The 5′ LTR, bacterial selectable marker and mammalian selectable marker are as described above. The selective nucleic acid recovery element is as the proviral recovery element described above.

The 3′ LTR contains the gene trapping cassette and lacks a functional LTR transcriptional promoter.

The gene trapping cassette can comprise from 5′ to 3′: (a) a nucleic acid sequence encoding at least one stop codon in each reading frame; (b) an internal ribosome entry site; and (c) a reporter sequence. The gene trapping cassette can further comprise, upstream of the stop codon sequences, a transcriptional splice acceptor nucleic acid sequence.

The inclusion of the IRES sequence in the gene trapping vectors of the present invention offers a key improvement over conventional gene trapping vectors. The IRES sequence allows the vector to land anywhere in the mature message to create a bicistronic transcript, this effectively increases the number of integration sites that will report promoters by a factor of at least 10. Although some of the vectors disclosed by U.S. Pat. No. 6,255,071 are intended for use in mammalian cells, with minor modification, most can be adepted for use in other cell types. Especially when specific packaging cells are used to generate viruses with a wide spectrum of infection.

Since these libraries are to be used for expression of Nux fusion proteins, a Nux coding sequence shall be present in the vector. Depending on specific configurations of the fusion protein, the Nux coding sequence could be either at the 5′- or 3′-end of the cloning site(s) for source DNA.

A normalized library is one constructed in a manner that increases the relative frequency of occurrence of rare clones while decreasing simultaneously the relative frequency of the occurrence of abundant clones. For teaching regarding the production of normalized libraries, see, e.g., Soares et al. (Soares, M. B. et al., 1994, Proc. Natl. Acad. Sci. USA 91:9228-9232, which is incorporated herein by reference in its entirety). Alternative normalization procedures based upon biotinylated nucleotides may also be utilized.

Those of ordinary skill in the art will recognize that methods for vector construction and protein expression described above and/or provided in the examples are examplary. It should be understood that there are other techniques, vectors, and cell lines that could be implemented for constructing and expressing proteins or fragments thereof in either procaryotic or eukaryotic systems. The preferred embodiment disclosed herein does not limit the scope of the invention. There are a variety of alternative techniques and procedures available to those with ordinary skill in the art that would permit one to perform modifications on the present invention. It is also well-known in the art that commercially available kits allow the modification and incorporation of the present invention. It is further recognized that those with ordinary skill in the art could employ any of a number of known techniques to modify the nucleic acid molecules of the present invention, in vitro or in vivo, and develop them further by established protocols for gene transfer and expression.

Screen methods

Although examplary mammalian cell complementation screening methods are described herein, it should be understood that many aspects of the described methods can be easily adapted for use in other cell types, which will be apparent to the person of ordinary skill in the art. Complementation screens in certain other cell types, especially in yeast, are well-known in the art. A classic example is genetic analysis of the cell cycle in the budding yeast S. cerevisiae (see review by Hartwell, L. H., Twenty-five years of cell cycle genetics, in Genetics 129: 975-980, 1991). Associated technologies such as yeast tranformation and overexpression of heterologous genes in yeast are well-known in the art and will not be addressed furher. Furthermore, knowledge based on yeast complementation screens has been adapted for use in cross-species complementation screens, for example, in yeast for plant (Arabidopsis) genes (Gietz, D. et al., Nucl. Acids Res. 20: 1425, 1992; Schiestl, R. H. and Gietz, R. D., Curr. Genet. 16: 339-334, 1989), the details of which will not be discussed further.

Nevertheless, complementation screens in mammalian cells constitute one of the most important aspects of the invention. Such complementation screen methods can include, for example, a method for identification of a nucleic acid sequence whose expression complements a cellular phenotype, comprising: (a) infecting a mammalian cell exhibiting the cellular phenotype with a, for example, retrovirus particle derived from a cDNA or gDNA-containing retroviral vector of the invention, or, alternatively, transfecting such a cell with a pEHRE vector of the invention wherein, depending on the vector, upon infection an integrated retroviral provirus is produced or upon transfection an episomal sequence is established, and the cDNA or GDNA sequence is expressed; and (b) analyzing the cell for the phenotype, so that suppression of the phenotype identifies a nucleic acid sequence which complements the cellular phenotype. Specifically, when a Nux-fusion protein is expressed at the presence of P-Cub-X-RM, interaction between P and the polypeptide encoded as a Nux-fusion will result in the generation of X-RM, which can then be detected depending on the specific nature of the reportermoiety and the nature of the amino acid X. Phenotypic differences between an uncleaved and cleaved X-RM shall allow selection of cells comprising cleaved X-RM.

Isolation and Characterization of Positive Clones

The vectors used may also facilitate the cloning and further characterization of the encoded polypeptide in the selected cell(s). Such methods utilize the proviral excision and the proviral recovery elements described above.

In one embodiment of such a method, the proviral excision element comprises a loxP recombination site present in two copies within the integrated provirus, and the proviral recovery element comprises a lacO site, present in the provirus between the two loxP sites. In this embodiment, the loxP sites are cleaved by a Cre recombinase enzyme, yielding an excised provirus which, upon excision, becomes circularized. The excised, circular provirus, which contains the lacO site is recovered from the complex mixture of recipient cell genomic nucleic acid by lac repressor affinity purification. Such an affinity purification is made possible by the fact that the lacO nucleic acid specifically binds to the lac repressor protein.

In an alternative embodiment, the excised provirus is amplified in order to increase its rescue efficiency. For example, the excised provirus can further comprise an SV40 origin of replication such that in vivo amplification of the excised provirus can be accomplished via delivery of large T antigen. The delivery can be made at the time of recombinase administration, for example.

In another alternative embodiment, the excised provirus may be recovered by use of a Cre recombinase. For example, the isolated DNA is fragmented to a controlled size. The provirus containing fragments are isolated via LacO/LacI. Following IPTG elution, circularization of the provirus can be accomplished by treatment with purified recombinase. The person skilled in the art will be able to anticipate other methods to isolate and characterize nucleic acids from selected cells.

4.7. Libraries and Screening Methods for the Screening of Agonists/Antagonists of known Specific-Binding Pair Interactions

The present invention provides methods to determine whether a test compound, or one of a number of test compounds, agonizes or antagonizes the binding of two proteins. When trying to identify an unknown compound which agonizes/antagonizes a particular interaction between two known polypeptides, one preferably will use a library of compounds and screen for members of such library that are capable of agonizing/antagonizing said interaction. This section shall outline how such libraries of compounds can be created, wherein these compounds may be polypeptides, peptides or small molecules. It is to be noted that in order to perform such screen, the libraries of compounds may have to be isolated further from the means used to prepare the library, such as peptides from a packaged display library, and be introduced into the host cells employed to screen for agonistic/antagonistic effects on the cleavage of a reporter moiety from a P1-Cub-X-RM polypeptide. The person skilled in the art will be able to anticipate methods to perform such isolation and introduction into cells.

A. Variegated Peptide Display

Variegated peptide libraries can be generated by any of a number of methods, and, though not limited by, preferably exploit recent trends in the preparation of chemical libraries. The library can be prepared, for example, by either synthetic or biosynthetic approaches, and screened for activity in an agonist/antagonist screen in a variety of assay formats. As used herein, “variegated” refers to the fact that a population of peptides is characterized by having a peptide sequence which differ from one member of the library to the next. For example, in a given peptide library of n amino acids in length, the total number of different peptide sequences in the library is given by the product of where each nn represents the number different amino acid residues occurring at position n of the peptide. In a preferred embodiment of the present invention, the peptide display collectively produces a peptide library including at least 96 to 10⁷ different peptides, so that diverse peptides may be simultaneously assayed for the ability to agonize/antagonize an interaction.

Peptide libraries are systems which simultaneously display a highly diverse and numerous collection of peptides. These peptides may be presented in solution (Houghten (1992) Biotechniques 13:412-421), or on beads (Lam (1991) Nature 354:82-84), chips (Fodor (1993) Nature 364:555-556), bacteria (Ladner U.S. Ser. No. 5,223,409), spores (Ladner U.S. Ser. No. '409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; and Ladner U.S. Ser. No. '409).

In one embodiment, the peptide library is derived to express a combinatorial library of peptides which are not based on any known sequence, nor derived from cDNA. That is, the sequences of the library are largely random. It will be evident that the peptides of the library may range in size from dipeptides to large proteins.

In another embodiment, the peptide library is derived to express a combinatorial library of peptides which are based at least in part on a known polypeptide sequence or a portion thereof (not a cDNA library). That is, the sequences of the library is semi-random, being derived by combinatorial mutagenesis of a known sequence(s). See, for example, Ladner et al. PCT publication WO 90/02909; Garrard et al., PCT publication WO 92/09690; Marks et al. (1992) J. Biol. Chem. 267:16007-16010; Griffths et al. (1993) EMBO J 12:725-734; Clackson et al. (1991) Nature 352:624-628; and Barbas et al. (1992) PNAS 89:4457-4461. Accordingly, polypeptide(s) can be mutagenized by standard techniques to derive a variegated library of polypeptide sequences which can further be screened for agonists and/or antagonists.

In still another embodiment, the combinatorial polypeptides are produced from a cDNA library.

Depending on size, the combinatorial peptides of the library can be generated as is, or can be incorporated into larger fusion proteins. The fusion protein can provide, for example, stability against degradation or denaturation, as well as a secretion signal if secreted. In an exemplary embodiment, the polypeptide library is provided as part of thioredoxin fusion proteins (see, for example, U.S. Pat. Nos. 5,270,181 and 5,292,646; and PCT publication WO94/02502). The combinatorial peptide can be attached on the terminus of the thioredoxin protein, or, for short peptide libraries, inserted into the so-called active loop.

In preferred embodiments, the combinatorial polypeptides are in the range of 3-100 amino acids in length, more preferably at least 5-50, and even more preferably at least 10, 13, 15, 20 or 25 amino acid residues in length. Preferably, the polypeptides of the library are of uniform length. It will be understood that the length of the combinatorial peptide does not reflect any extraneous sequences which may be present in order to facilitate expression, e.g., such as signal sequences or invariant portions of a fusion protein.

i) Biosynthetic Peptide Libraries

The harnessing of biological systems for the generation of peptide diversity is now a well established technique which can be exploited to generate the peptide libraries of the subject method. The source of diversity is the combinatorial chemical synthesis of mixtures of oligonucleotides. Oligonucleotide synthesis is a well-characterized chemistry that allows tight control of the composition of the mixtures created. Degenerate DNA sequences produced are subsequently placed into an appropriate genetic context for expression as peptides.

There are two principal ways in which to prepare the required degenerate mixture. In one method, the DNAs are synthesized a base at a time. When variation is desired at a base position dictated by the genetic code a suitable mixture of nucleotides is reacted with the nascent DNA, rather than the pure nucleotide reagent of conventional polynucleotide synthesis. The second method provides more exact control over the amino acid variation. First, trinucleotide reagents are prepared, each trinucleotide being a codon of one (and only one) of the amino acids to be featured in the peptide library. When a particular variable residue is to be synthesized, a mixture is made of the appropriate trinucleotides and reacted with the nascent DNA. Once the necessary “degenerate” DNA is complete, it must be joined with the DNA sequences necessary to assure the expression of the peptide, as discussed in more detail below, and the complete DNA construct must be introduced into the cell.

Whatever the method may be for generating diversity at the codon level, chemical synthesis of a degenerate gene sequence can be carried out in an automatic DNA synthesizer, and the synthetic genes can then be ligated into an appropriate gene for expression. The purpose of a degenerate set of genes is to provide, in one mixture, all of the sequences encoding the desired set of potential test peptide sequences. The synthesis of degenerate oligonucleotides is well known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Because the number of different peptides one can create by this combination approach can be huge, and because the expectation is that peptides with the appropriate structural characteristics to agonize/antagonize an interaction will be rare in the total population of the library,it may be advantageous to prescreen a peptide library for binding to one member of a specific-binding pair, where an agonist or antagonist is sought for the interaction of this specific-binding pair, and subsequently only introduce those peptides that bind to one member into a screen involving the interaction. Several strategies for selecting peptide ligands for a single protein from a library have been described in the art and are applicable to certain embodiments of the present method.

In one embodiment, a variegated peptide library can be expressed by a population of display packages to form a peptide display library. With respect to the display package on which the variegated peptide library is manifest, it will be appreciated from the discussion provided herein that the display package will often preferably be able to be (i) genetically altered to encode a test peptide, (ii) maintained and amplified in culture, (iii) manipulated to display the peptide in a manner permitting the peptide to interact with a member of a specific binding pair during an affinity separation step, and (iv) affinity separated while retaining the peptide-encoding gene such that the sequence of the peptide can be obtained. In preferred embodiments, the display remains viable after affinity separation.

Ideally, the display package comprises a system that allows the sampling of very large variegated peptide display libraries, rapid sorting after each affinity separation round, and easy isolation of the peptide-encoding gene from purified display packages. The most attractive candidates for this type of screening are prokaryotic organisms and viruses, as they can be amplified quickly, they are relatively easy to manipulate, and large number of clones can be created. Preferred display packages include, for example, vegetative bacterial cells, bacterial spores, and most preferably, bacterial viruses (especially DNA viruses). However, the present invention also contemplates the use of eukaryotic cells, including yeast and their spores, as potential display packages.

In addition to commercially available kits for generating phage display libraries (e.g. the Pharmacia Recombinant Phage Peptide System, catalog no. 27-9400-01; and the Stratagene SurfZAPTM phage display kit, catalog no. 240612), examples of methods and reagents particularly amenable for use in generating the variegated peptide display library of the present method can be found in, for example, the Ladner et al. U.S. Pat. No. 5,223,409; the Kang et al. International Publication No. WO 92/18619; the Dower et al. International Publication No. WO 91/17271; the Winter et al. International Publication WO 92/20791; the Markland et al. International Publication No. WO 92/15679; the Breitling et al. International Publication WO 93/01288; the McCafferty et al. International Publication No. WO 92/01047; the Garrard et al. International Publication No. WO 92/09690; the Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982.

When the display is based on a bacterial cell, or a phage which is assembled periplasmically, the display means of the package will comprise at least two components. The first component is a secretion signal which directs the recombinant peptide to be localized on the extracellular side of the cell membrane (of the host cell when the display package is a phage). This secretion signal is characteristically cleaved off by a signal peptidase to yield a processed, “mature” peptide. The second component is a display anchor protein which directs the display package to associate the peptide with its outer surface. As described below, this anchor protein can be derived from a surface or coat protein native to the genetic package.

When the display package is a bacterial spore, or a phage whose protein coating is assembled intracellularly, a secretion signal directing the peptide to the inner membrane of the host cell is unnecessary. In these cases, the means for arraying the variegated peptide library comprises a derivative of a spore or phage coat protein amenable for use as a fusion protein.

In the instance wherein the display package is a phage, the cloning site for the test peptide sequences in the phagemid should be placed so that it does not substantially interfere with normal phage function. One such locus is the intergenic region as described by Zinder and Boeke, (1982) Gene 19:1-10. In an illustrative embodiment comprising an M13 phage display library, the test peptide sequence is preferably expressed at an equal or higher-level than the HL-cpIII product (described below) to maintain a sufficiently high VL concentration in the periplasm and provide efficient assembly (association) of VL with VH chains. For instance, a phagemid can be constructed to encode, as separate genes, both a VH/coat fusion protein and a VL chain. Under the appropriate induction, both chains are expressed and allowed to assemble in the periplasmic space of the host cell, the assembled peptide being linked to the phage particle by virtue of the VH chain being a portion of a coat protein fusion construct.

The number of possible peptides for a given library may, in certain instances, exceed 1012. To sample as many combinations as possible depends, in part, on the ability to recover large numbers of transformants. For phage with plasmid-like forms (as filamentous phage), electrotransformation provides an efficiency comparable to that of phage-transfection with in vitro packaging, in addition to a very high capacity for DNA input. This allows large amounts of vector DNA to be used to obtain very large numbers of transformants. The method described by Dower et al. (1988) Nucleic Acids Res., 16:6127-6145, for example, may be used to transform fd-tet derived recombinants at the rate of about 107 transformants/ug of ligated vector into E. coli (such as strain MC1061), and libraries may be constructed in fd-tet B1 of up to about 3×108 members or more. Increasing DNA input and making modifications to the cloning protocol within the ability of the skilled artisan may produce increases of greater than about 10-fold in the recovery of transformants, providing libraries of up to 1010 or more recombinants.

As will be apparent to those skilled in the art, in embodiments wherein high affinity peptides are sought, an important criteria for the present selection method can be that it is able to discriminate between peptides of different affinity for a particular target, and preferentially enrich for the peptides of highest affinity. Applying the well known principles of affinity and valence, it is understood that manipulating the display package to be rendered effectively monovalent can allow affinity enrichment to be carried out for generally higher binding affinities (i.e. binding constants in the range of 106 to 1010 M-1) as compared to the broader range of affinities isolable using a multivalent display package. To generate the monovalent display, the natural (i.e. wild-type) form of the surface or coat protein used to anchor the peptide to the display can be added at a high enough level that it almost entirely eliminates inclusion of the peptide fusion protein in the display package. Thus, a vast majority of the display packages can be generated to include no more than one copy of the peptide fusion protein (see, for example, Garrad et al. (1991) Bio/Technology 9:1373-1377). In a preferred embodiment of a monovalent display library, the library of display packages will comprise no more than 5 to 10% polyvalent displays, and more preferably no more than 2% of the display will be polyvalent , and most preferably, no more than 1% polyvalent display packages in the population. The source of the wild-type anchor protein can be, for example, provided by a copy of the wild-type gene present on the same construct as the peptide fusion protein, or provided by a separate construct altogether.

a) Phage As Display Packages

Bacteriophage are attractive prokaryotic-related organisms for use in the subject method. Bacteriophage are excellent candidates for providing a display system of the variegated peptide library as there is little or no enzymatic activity associated with intact mature phage, and because their genes are inactive outside a bacterial host, rendering the mature phage particles metabolically inert. In general, the phage surface is a relatively simple structure. Phage can be grown easily in large numbers, they are amenable to the practical handling involved in many potential mass screening programs, and they carry genetic information for their own synthesis within a small, simple package. As the peptide gene is inserted into the phage genome, choosing the appropriate phage to be employed in the subject method will generally depend most on whether (i) the genome of the phage allows introduction of the peptide-encoding gene either by tolerating additional genetic material or by having replaceable genetic material; (ii) the virion is capable of packaging the genome after accepting the insertion or substitution of genetic material; and (iii) the display of the peptide on the phage surface does not disrupt virion structure sufficiently to interfere with phage propagation.

One concern presented with the use of phage is that the morphogenetic pathway of the phage determines the environment in which the peptide will have opportunity to fold. Periplasmically assembled phage are preferred as the displayed antibodies where the test peptide contains essential disulfides. However, in certain embodiments in which the display package forms intracellularly (e.g., where 1 phage are used), it has been demonstrated that the peptide may assume proper folding after the phage is released from the cell.

Another concern related to the use of phage, but also pertinent to the use of bacterial cells and spores as well, is that multiple infections could generate hybrid displays that carry the gene for one particular peptide yet have at least one or more different test peptides on their surfaces. Therefore, it can be preferable, though optional, to minimize this possibility by infecting cells with phage under conditions resulting in a low multiple infection. However, there may be circumstances in which high multiple-infection conditions would be desirable, such as to increase homologous recombination events between gene constructs encoding the peptide display in order to further expand the repertoire of the peptide display library.

For a given bacteriophage, the preferred display means is a protein that is present on the phage surface (e.g. a coat protein). Filamentous phage can be described by a helical lattice; isometric phage, by an icosahedral lattice. Each monomer of each major coat protein sits on a lattice point and makes defined interactions with each of its neighbors. Proteins that fit into the lattice by making some, but not all, of the normal lattice contacts are likely to destabilize the virion by aborting formation of the virion as well as by leaving gaps in the virion so that the nucleic acid is not protected. Thus in bacteriophage, unlike the cases of bacteria and spores, it is generally important to retain in the peptide fusion proteins those residues of the coat protein that interact with other proteins in the virion. For example, when using the M13 cpVIII protein, the entire mature protein will generally be retained with the peptide fragment being added to the N-terminus of cpVIII, while on the other hand it can suffice to retain only the last 100 carboxy terminal residues (or even fewer) of the M13 cpIII coat protein in the peptide fusion protein.

Under the appropriate induction, the peptide library is expressed and allowed to assemble in the bacterial cytoplasm, such as when the 1 phage is employed. The induction of the protein(s) may be delayed until some replication of the phage genome, synthesis of some of the phage structural-proteins, and assembly of some phage particles has occurred. The assembled protein chains then interact with the phage particles via the binding of the anchor protein on the outer surface of the phage particle. The cells are lysed and the phage bearing the library-encoded test peptides (that correspond to the specific library sequences carried in the DNA of that phage) are released and isolated from the bacterial debris.

To enrich for and isolate phage which contain cloned library sequences that encode a desired protein, and thus to ultimately isolate the nucleic acid sequences themselves, phage harvested from the bacterial debris are, for example, affinity purified. As described below, when a peptide which specifically binds a particular target protein is desired, the target protein can be used to retrieve phage displaying the desired peptide. The phage so obtained may then be amplified by infecting into host cells. Additional rounds of affinity enrichment followed by amplification may be employed until the desired level of enrichment is reached.

The enriched peptide-phage can also be screened with additional detection-techniques such as expression plaque (or colony) lift (see, e.g., Young and Davis, Science (1983) 222:778-782) whereby a labeled target protein is used as a probe. The phage obtained from the screening protocol are infected into cells, propagated, and the phage DNA isolated and sequenced, and/or recloned into a vector intended for gene expression in prokaryotes or eukaryotes to obtain larger amounts of the particular peptide selected.

In yet another embodiment, the peptide is also transported to an extra-cytoplasmic compartment of the host cell, such as the bacterial periplasm, but as a fusion protein with a viral coat protein. In this embodiment the desired protein (or one of its polypeptide chains if it is a multichain peptide) is expressed fused to a viral coat protein which is processed and transported to the cell inner membrane. Other chains, if present, are expressed with a secretion leader and thus are also transported to the periplasm or other intracellular by extra-cytoplasmic location. The chains present in the extra-cytoplasm then assemble into a complete test peptide. The assembled molecules become incorporated into the phage by virtue of their attachment to the phage coat protein as the phage extrude through the host membrane and the coat proteins assemble around the phage DNA. The phage bearing the test peptide may then be screened by affinity enrichment as described below.

1) Filamentous Phage

Filamentous bacteriophages, which include M13, f1, fd, If1, Ike, Xf, Pf1, and PB3, are a group of related viruses that infect bacteria. They are termed filamentous because they are long, thin particles comprised of an elongated capsule that envelopes the deoxyribonucleic acid (DNA) that forms the bacteriophage genome. The F pili filamentous bacteriophage (Ff phage) infect only gram-negative bacteria by specifically adsorbing to the tip of F pili, and include fd, f1 and M13.

Compared to other bacteriophage, filamentous phage in general are attractive for generating the peptide libraries of the subject method, and M13 in particular is especially attractive because: (i) the 3-D structure of the virion is known; (ii) the processing of the coat protein is well understood; (iii) the genome is expandable; (iv) the genome is small; (v) the sequence of the genome is known; (vi) the virion is physically resistant to shear, heat, cold, urea, guanidinium chloride, low pH, and high salt; (vii) the phage is a sequencing vector so that sequencing is especially easy; (viii) antibiotic-resistance genes have been cloned into the genome with predictable results (Hines et al. (1980) Gene 11:207-218); (ix) it is easily cultured and stored, with no unusual or expensive media requirements for the infected cells, (x) it has a high burst size, each infected cell yielding 100 to 1000 M13 progeny after infection; and (xi) it is easily harvested and concentrated (Salivar et al. (1964) Virology 24: 359-371). The entire life cycle of the filamentous phage M13, a common cloning and sequencing vector, is well understood. The genetic structure of M13 is well known, including the complete sequence (Schaller et al. in The Single-Stranded DNA Phages eds. Denhardt et al. (NY: CSHL Press, 1978)), the identity and function of the ten genes, and the order of transcription and location of the promoters, as well as the physical structure of the virion (Smith et al. (1985) Science 228:1315-1317; Raschad et al. (1986) Microbiol Dev 50:401-427; Kuhn et al. (1987) Science 238:1413-1415; Zimmerman et al. (1982) J Biol Chem 257:6529-6536; and Banner et al. (1981) Nature 289:814-816). Because the genome is small (6423 bp), cassette mutagenesis is practical on RF M13 (Current Protocols in Molecular Biology, eds. Ausubel et al. (NY: John Wiley & Sons, 1991)), as is single-stranded oligonucleotide directed mutagenesis (Fritz et al. in DNA Cloning, ed by Glover (Oxford, UK: IRC Press, 1985)). M13 is a plasmid and transformation system in itself, and an ideal sequencing vector. M13 can be grown on Rec? strains of E. coli. The M13 genome is expandable (Messing et al. in The Single-Stranded DNA Phages, eds Denhardt et al. (NY: CSHL Press, 1978) pages 449-453; and Fritz et al., supra) and M13 does not lyse cells. Extra genes can be inserted into M13 and will be maintained in the viral genome in a stable manner.

The mature capsule or Ff phage is comprised of a coat of five phage-encoded gene products: cpVIII, the major coat protein product of gene VIII that forms the bulk of the capsule; and four minor coat proteins, cpIII and cpIV at one end of the capsule and cpVII and cpIX at the other end of the capsule. The length of the capsule is formed by 2500 to 3000 copies of cpVIII in an ordered helix array that forms the characteristic filament structure. The gene III-encoded protein (cpIII) is typically present in 4 to 6 copies at one end of the capsule and serves as the receptor for binding of the phage to its bacterial host in the initial phase of infection. For detailed reviews of Ff phage structure, see Rasched et al., Microbiol. Rev., 50:401?427 (1986); and Model et al., in The Bacteriophages, Volume 2, R. Calendar, Ed., Plenum Press, pp. 375?456 (1988).

The phage particle assembly involves extrusion of the viral genome through the host cell's membrane. Prior to extrusion, the major coat protein cpVIII and the minor coat protein cpIII are synthesized and transported to the host cell's membrane. Both cpVIII and cpIII are anchored in the host cell membrane prior to their incorporation into the mature particle. In addition, the viral genome is produced and coated with cpV protein. During the extrusion process, cpV-coated genomic DNA is stripped of the cpV coat and simultaneously recoated with the mature coat proteins.

Both cpIII and cpVIII proteins include two domains that provide signals for assembly of the mature phage particle. The first domain is a secretion signal that directs the newly synthesized protein to the host cell membrane. The secretion signal is located at the amino terminus of the polypeptide and targets the polypeptide at least to the cell membrane. The second domain is a membrane anchor domain that provides signals for association with the host cell membrane and for association with the phage particle during assembly. This second signal for both cpVIII and cpIII comprises at least a hydrophobic region for spanning the membrane.

The 50 amino acid mature gene VIII coat protein (cpVIII) is synthesized as a 73 amino acid precoat (Ito et al. (1979) PNAS 76:1199-1203). The cpVIII protein has been extensively studied as a model membrane protein because it can integrate into lipid bilayers such as the cell membrane in an asymmetric orientation with the acidic amino terminus toward the outside and the basic carboxy terminus toward the inside of the membrane. The first 23 amino acids constitute a typical signal-sequence which causes the nascent polypeptide to be inserted into the inner cell membrane. An E. coli signal peptidase (SP?I) recognizes amino acids 18, 21, and 23, and, to a lesser extent, residue 22, and cuts between residues 23 and 24 of the precoat (Kuhn et al. (1985) J. Biol. Chem. 260:15914-15918; and Kuhn et al. (1985) J. Biol. Chem. 260:15907-15913). After removal of the signal sequence, the amino terminus of the mature coat is located on the periplasmic side of the inner membrane; the carboxy terminus is on the cytoplasmic side. About 3000 copies of the mature coat protein associate side-by-side in the inner membrane.

The sequence of gene VIII is known, and the amino acid sequence can be encoded on a synthetic gene. Mature gene VIII protein makes up the sheath around the circular ssDNA. The gene VIII protein can be a suitable anchor protein because its location and orientation in the virion are known (Banner et al. (1981) Nature 289:814-816). Preferably, the test peptide is attached to the amino terminus of the mature M13 coat protein to generate the phage display library. As set out above, manipulation of the concentration of both the wild-type cpVIII and test peptide/cpVIII fusion in an infected cell can be utilized to decrease the avidity of the display and thereby enhance the detection of high affinity antibodies directed to the target epitope(s).

Another vehicle for displaying the test peptide library is by expressing it as a domain of a chimeric gene containing part or all of gene III. When monovalent displays are required, expressing the test peptide as a fusion protein with cpIII can be a preferred embodiment, as manipulation of the ratio of wild-type gpIII to chimeric cpIII during formation of the phage particles can be readily controlled. This gene encodes one of the minor coat proteins of M13. In particular, the single-stranded circular phage DNA associates with about five copies of the gene III protein and is then extruded through the patch of membrane-associated coat protein in such a way that the DNA is encased in a helical sheath of protein (Webster et al. in The Single-Stranded DNA Phages, eds Dressler et al. (NY:CSHL Press, 1978).

Manipulation of the sequence of cpIII has demonstrated that the C-terminal 23 amino acid residue stretch of hydrophobic amino acids normally responsible for a membrane anchor function can be altered in a variety of ways and retain the capacity to associate with membranes. Ff phage-based expression vectors were first described in which the cpIII amino acid residue sequence was modified by insertion of polypeptide “epitopes” (Parmely et al., Gene (1988) 73:305-318; and Cwirla et al., PNAS (1990) 87:6378?6382) or an amino acid residue sequence defining a larger polypeptide domain (McCafferty et al., Science (1990) 348:552?554). It has been demonstrated that insertions into gene III can result in the production of novel protein domains on the virion outer surface. (Smith (1985) Science 228:1315-1317; and de la Cruz et al. (1988) J. Biol. Chem. 263:4318-4322). The test peptide-encoding gene may be fused to gene III at the site used by Smith and by de la Cruz et al., e.g., at a codon corresponding to another domain boundary or to a surface loop of the protein, or to the amino terminus of the mature protein.

Similar constructions could be made with other filamentous phage. Pf3 is a well known filamentous phage that infects Pseudomonas aerugenosa cells that harbor an IncP-I plasmid. The entire genome has been sequenced ((Luiten et al. (1985) J. Virol. 56:268-276) and the genetic signals involved in replication and assembly are known (Luiten et al. (1987) DNA 6:129-137). The major coat protein of PF3 is unusual in having no signal peptide to direct its secretion. The sequence has charged residues ASP-7, ARG-37, LYS-40, and PHE44 which is consistent with the amino terminus being exposed. Thus, to cause a test peptide to appear on the surface of Pf3, a tripartite gene can be constructed which comprises a signal sequence known to cause secretion in P. aerugenosa, fused in-frame to a gene fragment encoding the test peptide sequence, which is fused in-frame to DNA encoding the mature Pf3 coat protein. Optionally, DNA encoding a flexible linker of one to 10 amino acids is introduced between the test peptide fragment and the Pf3 coat-protein gene. This tripartite gene is introduced into P13. Once the signal sequence is cleaved off, the test peptide is in the periplasm and the mature coat protein acts as an anchor and phage-assembly signal.

2) Bacteriophage fX174

The bacteriophage fX174 is a very small icosahedral virus which has been thoroughly studied by genetics, biochemistry, and electron microscopy (see The Single Stranded DNA Phages (eds. Den hard et al. (NY:CSHL Press, 1978)). Three gene products of fX174 are present on the outside of the mature virion: F (cased), G (major spike protein, 60 copies per virion), and H (minor spike protein, 12 copies per virion). The G protein comprises 175 amino acids, while H comprises 328 amino acids. The F protein interacts with the single-stranded DNA of the virus. The proteins F, G, and H are translated from a single mRNA in the viral infected cells. As the virus is so tightly constrained because several of its genes overlap, fX174 is not typically used as a cloning vector due to the fact that it can accept very little additional DNA. However, mutations in the viral G gene (encoding the G protein) can be rescued by a copy of the wild-type G gene carried on a plasmid that is expressed in the same host cell (Chambers et al. (1982) Nuc Acid Res 10:6465-6473). In one embodiment, one or more stop codons are introduced into the G gene so that no G protein is produced from the viral genome. Nucleic acid encoding the variegated peptide library can then be fused with the nucleic acid sequence of the H gene. An amount of the viral G gene equal to the size of the test peptide gene fragment is eliminated from the fX174 genome, such that the size of the genome is ultimately unchanged. Thus, in host cells also transformed with a second plasmid expressing the wild-type G protein, the production of viral particles from the mutant virus is rescued by the exogenous G protein source. Where it is desirable that only one test peptide be displayed per *X174 particle (e.g., monovalent), the second plasmid can further include one or more copies of the wild-type H protein gene so that a mix of H and test peptide/H proteins will be predominated by the wild-type H upon incorporation into phage particles.

3) Large DNA Phage

Phage such as 1 or T4 have much larger genomes than do M13 or fX174, and have more complicated 3-D capsid structures than M13 or fPX174, with more coat proteins to choose from. In embodiments of the invention whereby the peptide library is processed and assembled into a functional form and associates with the bacteriophage particles within the cytoplasm of the host cell, bacteriophage 1 and derivatives thereof are examples of suitable vectors. The intracellular morphogenesis of phage 1 can potentially prevent protein domains that ordinarily contain disulfide bonds from folding correctly. However, variegated libraries expressing a population of functional antibodies, including both heavy and light chain variable regions, have been generated in 1 phage, indicating that disulfide bonds can be formed in the test peptide library. (Huse et al. (1989) Science 246:1275-1281; Mullinax et al. (1990) PNAS 87:8095-8099; and Pearson et al. (1991) PNAS 88:2432-2436). Such strategies take advantage of the rapid construction and efficient transformation abilities of 1 phage.

When used for expression of peptide sequences, library DNA sequences may be readily inserted into a 1 vector. For instance, variegated peptide libraries have been constructed by modification of 1 ZAP II (Short et al. (1988) Nuc Acid Res 16:7583) comprising inserting the peptide-encoding nucleic acid into the multiple cloning site of a 1 ZAP II vector (Huse et al. supra.).

b) Bacterial Cells as Display Packages

Recombinant peptides are able to cross bacterial membranes after the addition of bacterial leader sequences to the peptides (Better et al (1988) Science 240:1041-1043; and Skerra et al. (1988) Science 240:1038-1041). In addition, recombinant peptides have been fused to outer membrane proteins for surface presentation. Accordingly, one strategy for displaying test peptides on bacterial cells comprises generating a fusion protein by adding the test peptide to cell surface exposed portions of an integral outer membrane protein (Fuchs et al. (1991) Bio/Technology 9:1370-1372). In selecting a bacterial cell to serve as the display package, any well-characterized bacterial strain will typically be suitable, provided the bacteria may be grown in culture, engineered to display the peptide library on its surface, and is compatible with the particular affinity selection process practiced in the subject method. Among bacterial cells, the preferred display systems include Salmonella typhimurium, Bacillus subtilis, Pseudomonas aeruginosa, Vibrio cholerae, Klebsiella pneumonia, Neisseria gonorrhoeae, Neisseria meningitidis, Bacteroides nodosus, Moraxella bovis, and especially Escherichia coli. Many bacterial cell surface proteins useful in the present invention have been characterized, and works on the localization of these proteins and the methods of determining their structure include Benz et al. (1988) Ann Rev Microbiol 42: 359-393; Balduyck et al. (1985) Biol Chem Hoppe-Seyler 366:9-14; Ehrmann et al (1990) PNAS 87:7574-7578; Heijne et al. (1990) Protein Engineering 4:109-112; Ladner et al. U.S. Pat. No. 5,223,409; Ladner et al. WO88/06630; Fuchs et al. (1991) Bio/technology 9:1370-1372; and Goward et al. (1992) TIBS 18:136-140.

To further illustrate, the LamB protein of E coli is a well understood surface protein that can be used to generate a variegated library of test peptides (see, for example, Ronco et al. (1990) Biochemie 72:183-189; van der Weit et al. (1990) Vaccine 8:269-277; Charabit et al. (1988) Gene 70:181-189; and Ladner U.S. Pat. No. 5,222,409). LamB of E. coli is a porin for maltose and maltodextrin transport, and serves as the receptor for adsorption of bacteriophages 1 and K10. LamB is transported to the outer membrane if a functional N-terminal signal sequence is present (Benson et al. (1984) PNAS 81:3830-3834). As with other cell surface proteins, LamB is synthesized with a typical signal-sequence which is subsequently removed. Thus, the variegated peptide-encoding gene library can be cloned into the LamB gene such that the resulting library of fusion proteins comprise a portion of LamB sufficient to anchor the protein to the cell membrane with the test peptide portion oriented on the extracellular side of the membrane. Secretion of the extracellular portion of the fusion protein can be facilitated by inclusion of the LamB signal sequence, or other suitable signal sequence, as the N-terminus of the protein.

The E. coli LamB has also been expressed in functional form in S. typhimurium (Harkki et al. (1987) Mol Gen Genet 209:607-611), V. cholerae (Harkki et al. (1986) Microb Pathol 1:283-288), and K. pneumonia (Wehmeier et al. (1989) Mol Gen Genet 215:529-536), so that one could display a population of test peptides in any of these species as a fusion to E. coli LamB. Moreover, K. pneumonia expresses a maltoporin similar to LamB which could also be used. In P. aeruginosa, the D1 protein (a homologue of LamB) can be used (Trias et al. (1988) Biochem Biophys Acta 938:493-496). Similarly, other bacterial surface proteins, such as PAL, OmpA, OmpC, OmpF, PhoE, pilin, BtuB, FepA, FhuA, IutA, FecA and FhuE, may be used in place of LamB as a portion of the display means in a bacterial cell.

c) Bacterial Spores as Display Packages

Bacterial spores also have desirable properties as display package candidates in the subject method. For example, spores are much more resistant than vegetative bacterial cells or phage to chemical and physical agents, and hence permit the use of a great variety of affinity selection conditions. Also, Bacillus spores neither actively metabolize nor alter the proteins on their surface. However, spores have the disadvantage that the molecular mechanisms that trigger sporulation are less well worked out than is the formation of M13 or the export of protein to the outer membrane of E. coli, though such a limitation is not a serious detractant from their use in the present invention.

Bacteria of the genus Bacillus form endospores that are extremely resistant to damage by heat, radiation, desiccation, and toxic chemicals (reviewed by Losick et al. (1986) Ann Rev Genet 20:625-669). This phenomenon is attributed to extensive intermolecular cross-linking of the coat proteins. In certain embodiments of the subject method, such as those which include relatively harsh affinity separation steps, such spores can be the preferred display package. Endospores from the genus Bacillus are more stable than are, for example, exospores from Streptomyces. Moreover, Bacillus subtilis forms spores in 4 to 6 hours, whereas Streptomyces species may require days or weeks to sporulate. In addition, genetic knowledge and manipulation is much more developed for B. subtilis than for other spore-forming bacteria.

Viable spores that differ only slightly from wild-type are produced in B. subtilis even if any one of four coat proteins is missing (Donovan et al. (1987) J Mol Biol 196:1-10). Moreover, plasmid DNA is commonly included in spores, and plasmid encoded proteins have been observed on the surface of Bacillus spores (Debra et al. (1986) J Bacteriol 165:258-268). Thus, it can be possible during sporulation to express a gene encoding a chimeric coat protein comprising a test peptide of the variegated gene library, without interfering materially with spore formation.

To illustrate, several polypeptide components of B. subtilis spore coat (Donovan et al. (1987) J Mol Biol 196:1 -10) have been characterized. The sequences of two complete coat proteins and amino-terminal fragments of two others have been determined. Fusion of the test peptide sequence to cotC or cotD fragments is likely to cause the test peptide to appear on the spore surface. The genes of each of these spore coat proteins are preferred as neither cotC or cotD are post-translationally modified (see Lader et al. U.S. Pat. No. 5,223,409).

ii) Synthetic Peptide Libraries

In contrast to the recombinant methods, in vitro chemical synthesis provides a method for generating libraries of compounds, without the use of living organisms, that can be screened for ability to bind to a agonize/antagonize an interaction. Although in vitro methods have been used for quite some time in the pharmaceutical industry to identify potential drugs, recently developed methods have focused on rapidly and efficiently generating and screening large numbers of compounds and are particularly amenable to generating peptide libraries for use in the subject method. The various approaches to simultaneous preparation and analysis of large numbers of synthetic peptides (herein “multiple peptide synthesis” or “MPS”) each rely on the fundamental concept of synthesis on a solid support introduced by Merrifield in 1963 (Merrifield, R. B. (1963) J Am Chem Soc 85:2149-2154; and references cited in section I above). Generally, these techniques are not dependent on the protecting group or activation chemistry employed, although most workers today avoid Merrifield's original tBoc/Bz1 strategy in favor of the more mild Fmoc/tBu chemistry and efficient hydroxybenzotriazole-based coupling agents. Many types of solid matrices have been successfully used in MPS, and yields of individual peptides synthesized vary widely with the technique adopted (e.g., nanomoles to millimoles).

a) Multipin Synthesis

One form that the peptide library of the subject method can take is the multipin library format. Briefly, Geysen and co-workers (Geysen et al. (1984) PNAS 81:3998-4002) introduced a method for generating peptide by a parallel synthesis on polyacrylic acid-grated polyethylene pins arrayed in the microtitre plate format. In the original experiments, about 50 nmol of a single peptide sequence was covalently linked to the spherical head of each pin, and interactions of each peptide with receptor or antibody could be determined in a direct binding assay. The Geysen technique can be used to synthesize and screen thousands of peptides per week using the multipin method, and the tethered peptides may be reused in many assays. In subsequent work, the level of peptide loading on individual pins has been increased to as much as 2*mol/pin by grafting greater amounts of functionalized acrylate derivatives to detachable pin heads, and the size of the peptide library has been increased (Valerio et al. (1993) Int J Pept Protein Res 42:1-9). Appropriate linker moieties have also been appended to the pins so that the peptides may be cleaved from the supports after synthesis for assessment of purity and evaluation in competition binding or functional bioassays (Bray et al. (1990) Tetrahedron Lett 31:5811-5814; Valerio et al. (1991) Anal Biochem 197:168-177; Bray et al. (1991) Tetrahedron Lett 32:6163-6166).

More recent applications of the multipin method of MPS have taken advantage of the cleavable linker strategy to prepare soluble peptides (Maeji et al. (1990) J Immunol Methods 134:23-33; Gammon et al. (1991) J Exp Med 173:609-617; Mutch et al. (1991) Pept Res 4:132-137).

b) Divide-Couple-Recombine

In yet another embodiment, a variegated library of peptides can provide on a set of beads utilizing the strategy of divide-couple-recombine (see, e.g., Houghten (1985) PNAS 82:5131-5135; and U.S. Pat. Nos. 4,631,211; 5,440,016; 5,480,971). Briefly, as the name implies, at each synthesis step where degeneracy is introduced into the library, the beads are divided into as many separate groups to correspond to the number of different amino acid residues to be added that position, the different residues coupled in separate reactions, and the beads recombined into one pool for the next step.

In one embodiment, the divide-couple-recombine strategy can be carried out using the so-called “tea bag” MPS method first developed by Houghten, peptide synthesis occurs on resin that is sealed inside porous polypropylene bags (Houghten et al. (1986) PNAS 82:5131-5135). Amino acids are coupled to the resins by placing the bags in solutions of the appropriate individual activated monomers, while all common steps such as resin washing and *-amino group deprotection are performed simultaneously in one reaction vessel. At the end of the synthesis, each bag contains a single peptide sequence, and the peptides may be liberated from the resins using a multiple cleavage apparatus (Houghten et al. (1986) Int J Pept Protein Res 27:673-678). This technique offers advantages of considerable synthetic flexibility and has been partially automated (Beck-Sickinger et al. (1991) Pept Res 4:88-94). Moreover, soluble peptides of greater than 15 amino acids in length can be produced in sufficient quantities (>0.500*mol) for purification and complete characterization if desired.

Multiple peptide synthesis using the tea-bag approach is useful for the production of a peptide library, albeit of limited size, for screening the present method, as is illustrated by its use in a range of molecular recognition problems including antibody epitope analysis (Houghten et al. (1986) PNAS 82:5131-5135), peptide hormone structure-function studies (Beck-Sickinger et al. (1990) Int J Pept Protein Res 36:522-530; Beck-Sickinger et al. (1990) Eur J Biochem 194:449-456), and protein conformational mapping (Zimmerman et al. (1991) Eur J Biochem 200:519-528).

An exemplary synthesis of a set of mixed peptides having equimolar amounts of the twenty natural amino acid residues is as follows. Aliquots of five grams (4.65 mmols) of p-methylbenzhydrylamine hydrochloride resin (MBHA) are placed into twenty porous polypropylene bags. These bags are placed into a common container and washed with 1.0 liter of CH2Cl2 three times (three minutes each time), then again washed three times (three minutes each time) with 1.0 liter of 5 percent DIEA/CH2Cl2 (DIEA=diisopropylethylamine; CH2Cl2=DCM). The bags are then rinsed with DCM and placed into separate reaction vessels each containing 50 ml (0.56M) of the respective t-BOC-amino acid/DCM. N,N-Diisopropylcarbodiimide (DIPCDI; 25 ml; 1.12M) is added to each container, as a coupling agent. Twenty amino acid derivatives are separately coupled to the resin in 50/50 (v/v) DMF/DCM. After one hour of vigorous shaking, Gisen's picric acid test (Gisen (1972) Anal. Chem. Acta 58:248-249) is performed to determine the completeness of the coupling reaction. On confirming completeness of reaction, all of the resin packets are then washed with 1.5 liters of DMF and washed two more times with 1.5 liters of CH2Cl2. After rinsing, the resins are removed from their separate packets and admixed together to form a pool in a common bag. The resulting resin mixture is then dried and weighed, divided again into 20 equal portions (aliquots), and placed into 20 further polypropylene bags (enclosed).

In a common reaction vessel the following steps are carried out: (1) deprotection is carried out on the enclosed aliquots for thirty minutes with 1.5 liters of 55 percent TFA/DCM; and 2) neutralization is carried out with three washes of 1.5 liters each of 5 percent DIEA/DCM. Each bag is placed in a separate solution of activated t-BOC-amino acid derivative and the coupling reaction carried out to completion as before. All coupling reactions are monitored using the above quantitative picric acid assay.

Next, the bags are opened and the resulting t-BOC-protected dipeptide resins are mixed together to form a pool, aliquots are made from the pool, the aliquots are enclosed, deprotected and further reactions are carried out. This process can be repeated any number of times yielding at each step an equimolar representation of the desired number of amino acid residues in the peptide chain. The principal process steps are conveniently referred to as a divide-couple-recombine synthesis.

After a desired number of such couplings and mixtures are carried out, the polypropylene bags are kept separated to here provide the twenty sets having the amino-terminal residue as the single, predetermined residue, with, for example, positions 2-4 being occupied by equimolar amounts of the twenty residues. To prepare sets having the single, predetermined amino acid residue at other than the amino-terminus, the contents of the bags are not mixed after adding a residue at the desired, predetermined position. Rather, the contents of each of the twenty bags are separated into 20 aliquots, deprotected and then separately reacted with the twenty amino acid derivatives. The contents of each set of twenty bags thus produced are thereafter mixed and treated as before-described until the desired oligopeptide length is achieved.

c) Multiple Peptide Synthesis through Coupling of Amino Acid Mixtures

Simultaneous coupling of mixtures of activated amino acids to a single resin support has been used as a multiple peptide synthesis strategy on several occasions (Geysen et al. (1986) Mol Immunol 23:709-715; Tjoeng et al. (1990) Int J Pept Protein Res 35:141-146; Rutter et al. (1991) U.S. Pat. No. 5,010,175; Birkett et al. (1991) Anal Biochem 196:137-143; Petithory et al. (1991) PNAS 88:11510-11514) and can have applications in the subject method. For example, four to seven analogs of the magainin 2 and angiotensinogen peptides were successfully synthesized and resolved in one HPLC purification after coupling a mixture of amino acids at a single position in each sequence (Tjoeng et al. (1990) Int J Pept Protein Res 35:141-146). This approach has also been used to prepare degenerate peptide mixtures for defining the substrate specificity of endoproteolytic enzymes (Birkett et al. (1991) Anal Biochem 196:137-143; Petithory et al. (1991) PNAS 88:11510-11514). In these experiments a series of amino acids was substituted at a single position within the substrate sequence. After proteolysis, Edman degradation was used to quantitate the yield of each amino acid component in the hydrolysis product and hence to evaluate the relative kcat/Km values for each substrate in the mixture.

However, it is noted that the operational simplicity of synthesizing many peptides by coupling monomer mixtures is offset by the difficulty in controlling the composition of the products. The product distribution reflects the individual; rate constants for the competing coupling reactions, with activated derivatives of sterically hindered residues such as valine or isoleucine adding at a significantly slower rate than glycine or alanine for example. The nature of the resin-bound component of the acylation reaction also influences the addition rate, and the relative rate constants for the formation of 400 dipeptides form the 20 genetically coded amino acids have been determined by Rutter and Santi (Rutter et al. (1991) U.S. Pat. No. 5,010,175). These reaction rates can be used to guide the selection of appropriate relative concentrations of amino acids in the mixture to favor more closely equimolar coupling yields.

d) Multiple Peptide Synthesis on Nontraditional Solid Supports

The search for innovative methods of multiple peptide synthesis has led to the investigation of alternative polymeric supports to the polystyrene-divinylbenzene matrix originally popularized by Merrifield. Cellulose, either in the form of paper disks (Blankemeyer-Menge et al. (1988) Tetrahedron Lett 29-5871-5874; Frank et al. (1988) Tetrahedron 44:6031-6040; Eichler et al. (1989) Collect Czech Chem Commun 54:1746-1752; Frank, R. (1993) Bioorg Med Chem Lett 3:425-430) or cotton fragments (Eichler et al. (1991) Pept Res 4:296-307; Schmidt et al. (1993) Bioorg Med Chem Lett 3:441-446) has been successfully functionalized for peptide synthesis. Typical loadings attained with cellulose paper range from 1 to 3*mol/cm2, and HPLC analysis of material cleaved from these supports indicates a reasonable quality for the synthesized peptides. Alternatively, peptides may be synthesized on cellulose sheets via non-cleavable linkers and then used in ELISA-based binding studies (Frank, R. (1992) Tetrahedron 48:9217-9232). The porous, polar nature of this support may help suppress unwanted nonspecific protein binding effects. By controlling the volume of activated amino acids and other reagents spotted on the paper, the number of peptides synthesized at discrete locations on the support can be readily varied. In one convenient configuration spots are made in an 8×12 microtiter plate format. Frank has used this technique to map the dominant epitopes of an antiserum raised against a human cytomegalovirus protein, following the overlapping peptide screening (Pepscan) strategy of Geysen (Frank, R. (1992) Tetrahedron 48:9217-9232). Other membrane-like supports that may be used for multiple solid-phase synthesis include polystyrene-grafted polyethylene films (Berg et al. (1989) J Am Chem Soc 111:8024-8026).

e) Combinatorial Libraries by Light-Directed, Spatially Addressable Parallel Chemical Synthesis

A scheme of combinatorial synthesis in which the identity of a compound is given by its locations on a synthesis substrate is termed a spatially-addressable synthesis. In one embodiment, the combinatorial process is carried out by controlling the addition of a chemical reagent to specific locations on a solid support (Dower et al. (1991) Annu Rep Med Chem 26:271-280; Fodor, S. P. A. (1991) Science 251:767; Pirrung et al. (1992) U.S. Pat. No. 5,143,854; Jacobs et al. (1994) Trends Biotechnol 12:19-26). The technique combines two well-developed technologies: solid-phase peptide synthesis chemistry and photolithography. The high coupling yields of Merrifield chemistry allow efficient peptide synthesis, and the spatial resolution of photolithography affords miniaturization. The merging of these two technologies is done through the use of photolabile amino protecting groups in the Merrifield synthetic procedure.

The key points of this technology are illustrated in Gallop et al. (1994) J Med Chem 37:1233-1251. A synthesis substrate is prepared for amino acid coupling through the covalent attachment of photolabile nitroveratryloxycarbonyl (NVOC) protected amino linkers. Light is used to selectively activate a specified region of the synthesis support for coupling. Removal of the photolabile protecting groups by lights (deprotection) results in activation of selected areas. After activation, the first of a set of amino acids, each bearing a photolabile protecting group on the amino terminus, is exposed to the entire surface. Amino acid coupling only occurs in regions that were addressed by light in the preceding step. The solution of amino acid is removed, and the substrate is again illuminated through a second mask, activating a different region for reaction with a second protected building block. The pattern of masks and the sequence of reactants define the products and their locations. Since this process utilizes photolithography techniques, the number of compounds that can be synthesized is limited only by the number of synthesis sites that can be addressed with appropriate resolution. The position of each compound is precisely known; hence, its interactions with other molecules can be directly assessed. Such other molecules can be labeled with a fluorescent reporter group to facilitate the identification of specific interactions with individual members of the matrix.

In a light-directed chemical synthesis, the products depend on the pattern of illumination and on the order of addition of reactants. By varying the lithographic patterns, many different sets of test peptides can be synthesized in the same number of steps; this leads to the generated of many different masking strategies.

f) Encoded Combinatorial Libraries

In yet another embodiment, the subject method utilizes a peptide library provided with an encoded tagging system. A recent improvement in the identification of active compounds from combinatorial libraries employs chemical indexing systems using tags that uniquely encode the reaction steps a given bead has undergone and, by inference, the structure it carries. Conceptually, this approach mimics phage display libraries above, where activity derives from expressed peptides, but the structures of the active peptides are deduced from the corresponding genomic DNA sequence. The first encoding of synthetic combinatorial libraries employed DNA as the code. Two forms of encoding have been reported: encoding with sequenceable bio-oligomers (e.g., oligonucleotides and peptides), and binary encoding with non-sequenceable tags.

1) Tagging with sequenceable bio-oligomers

The principle of using oligonucleotides to encode combinatorial synthetic libraries was described in 1992 (Brenner et al. (1992) PNAS 89:5381-5383), and an example of such a library appeared the following year (Needles et al. (1993) PNAS 90:10700-10704). A combinatorial library of nominally 77 (=823,543) peptides composed of all combinations of Arg, Gln, Phe, Lys, Val, D-Val and Thr (three-letter amino acid code), each of which was encoded by a specific dinucleotide (TA, TC, CT, AT, TT, CA and AC, respectively), was prepared by a series of alternating rounds of peptide and oligonucleotide synthesis on solid support. In this work, the amine linking functionality on the bead was specifically differentiated toward peptide or oligonucleotide synthesis by simultaneously preincubating the beads with reagents that generate protected OH groups for oligonucleotide synthesis and protected NH2 groups for peptide synthesis (here, in a ratio of 1:20). When complete, the tags each consisted of 69-mers, 14 units of which carried the code. The bead-bound library was incubated with a fluorescently labeled antibody, and beads containing bound antibody that fluoresced strongly were harvested by fluorescence-activated cell sorting (FACS). The DNA tags were amplified by PCR and sequenced, and the predicted peptides were synthesized. Following the such techniques, the peptide libraries can be derived for use in the subject method and screened.

It is noted that an alternative approach useful for generating nucleotide-encoded synthetic peptide libraries employs a branched linker containing selectively protected OH and NH2 groups (Nielsen et al. (1993) J Am Chem Soc 115:9812-9813; and Nielsen et al. (1994) Methods Compan Methods Enzymol 6:361-371). This approach requires that equimolar quantities of test peptide and tag co-exist, though this may be a potential complication in assessing biological activity, especially with nucleic acid based targets.

The use of oligonucleotide tags permits exquisitely sensitive tag analysis. Even so, the method requires careful choice of orthogonal sets of protecting groups required for alternating co-synthesis of the tag and the library member. Furthermore, the chemical lability of the tag, particularly the phosphate and sugar anomeric linkages, may limit the choice of reagents and conditions that can be employed for the synthesis on non-oligomeric libraries. In preferred embodiments, the libraries employ linkers permitting selective detachment of the test peptide library member for bioassay, in part because the tags are potentially susceptible to biodegradation.

Peptides themselves have been employed as tagging molecules for combinatorial libraries. Two exemplary approaches are described in the art, both of which employ branched linkers to solid phase upon which coding and ligand strands are alternately elaborated. In the first approach (Kerr J M et al. (1993) J Am Chem Soc 115:2529-2531), orthogonality in synthesis is achieved by employing acid-labile protection for the coding strand and base-labile protection for the ligand strand.

In an alternative approach (Nikolaiev et al. (1993) Pept Res 6:161-170), branched linkers are employed so that the coding unit and the test peptide are both attached to the same functional group on the resin. In one embodiment, a linker can be placed between the branch point and the bead so that cleavage releases a molecule containing both code and ligand (Ptek et al. (1991) Tetrahedron Lett 32:3891-3894). In another embodiment, the linker can be placed so that the test peptide can be selectively separated from the bead, leaving the code behind. This last construct is particularly valuable because it permits screening of the test peptide without potential interference, or biodegradation, of the coding groups. Examples in the art of independent cleavage and sequencing of peptide library members and their corresponding tags has confirmed that the tags can accurately predict the peptide structure.

It is noted that peptide tags are more resistant to decomposition during ligand synthesis than are oligonucleotide tags, but they must be employed in molar ratios nearly equal to those of the ligand on typical 130 mm beads in order to be successfully sequenced. As with oligonucleotide encoding, the use of peptides as tags requires complex protection/deprotection chemistries.

2) Non-Sequenceable Tagging: Binary Encoding

An alternative form of encoding the test peptide library employs a set of non-sequenceable electrophoric tagging molecules that are used as a binary code (Ohlmeyer et al. (1993) PNAS 90:10922-10926). Exemplary tags are haloaromatic alkyl ethers that are detectable as their tetramethylsilyl ethers at less than femtomolar levels by electron capture gas chromatography (ECGC). Variations in the length of the alkyl chain, as well as the nature and position of the aromatic halide substituents, permit the synthesis of at least 40 such tags, which in principle can encode 240 (e.g., upwards of 1012) different molecules. In the original report (Ohlmeyer et al., supra) the tags were bound to about 1% of the available amine groups of a peptide library via a photocleavable O-nitrobenzyl linker. This approach is convenient when preparing combinatorial libraries of peptides or other amine-containing molecules. A more versatile system has, however, been developed that permits encoding of essentially any combinatorial library. Here, the ligand is attached to the solid support via the photocleavable linker and the tag is attached through a catechol ether linker via carbene insertion into the bead matrix (Nestler et al. (1994) J Org Chem 59:4723-4724). This orthogonal attachment strategy permits the selective detachment of library members for bioassay in solution and subsequent decoding by ECGC after oxidative detachment of the tag sets.

Binary encoding with electrophoric tags has been particularly useful in defining selective interactions of substrates with synthetic receptors (Borchardt et al. (1994) J Am Chem Soc 116:373-374), and model systems for understanding the binding and catalysis of biomolecules. Even using detailed molecular modeling, the identification of the selectivity preferences for synthetic receptors has required the manual synthesis of dozens of potential substrates. The use of encoded libraries makes it possible to rapidly examine all the members of a potential binding set. The use of binary-encoded libraries has made the determination of binding selectivities so facile that structural selectivity has been reported for four novel synthetic macrobicyclic and tricyclic receptors in a single communication (Wennemers et al. (1995) J Org Chem 60:1108-1109; and Yoon et al. (1994) Tetrahedron Lett 35:8557-8560) using the encoded library mentioned above. Similar facility in defining specificity of interaction would be expected for many other biomolecules.

Although the several amide-linked libraries in the art employ binary encoding with the electrophoric tags attached to amine groups, attaching these tags directly to the bead matrix provides far greater versatility in the structures that can be prepared in encoded combinatorial libraries. Attached in this way, the tags and their linker are nearly as unreactive as the bead matrix itself. Two binary-encoded combinatorial libraries have been reported where the electrophoric tags are attached directly to the solid phase (Ohlmeyer et al. (1995) PNAS 92:6027-6031) and provide guidance for generating the subject peptide library. Both libraries were constructed using an orthogonal attachment strategy in which the library member was linked to the solid support by a photolabile linker and the tags were attached through a linker cleavable only by vigorous oxidation. Because the library members can be repetitively partially photoeluted from the solid support, library members can be utilized in multiple assays. Successive photoelution also permits a very high throughput iterative screening strategy: first, multiple beads are placed in 96-well microtiter plates; second, ligands are partially detached and transferred to assay plates; third, a bioassay identifies the active wells; fourth, the corresponding beads are rearrayed singly into new microtiter plates; fifth, single active compounds are identified; and sixth, the structures are decoded.

The above approach was employed in screening for carbonic anhydrase (CA) binding and identified compounds which exhibited nanomolar affinities for CA. Unlike sequenceable tagging, a large number of structures can be rapidly decoded from binary-encoded libraries (a single ECGC apparatus can decode 50 structures per day). Thus, binary-encoded libraries can be used for the rapid analysis of structure-activity relationships and optimization of both potency and selectivity of an active series. The synthesis and screening of large unbiased binary encoded peptide libraries for lead identification, followed by preparation and analysis of smaller focused libraries for lead optimization, offers a particularly powerful approach to drug discovery using the subject method.

iii) Nucleic Acid Libraries

In another embodiment, the library is comprised of a variegated pool of nucleic acids, e.g. single or double-stranded DNA or ARNA. A variety of techniques are known in the art for generating screenable nucleic acid libraries which may be exploited in the present invention. In particular, many of the techniques described above for synthetic peptide libraries can be used to generate nucleic acid libraries of a variety of formats. For example, divide-couple-recombine techniques can be used in conjugation with standard nucleic acid synthesis techniques to generate bead immobilized nucleic acid libraries.

In another embodiment, solution libraries of nucleic acids can be generated which rely on PCR techniques to amplify for sequencing those nucleic acid molecules which agonize/antagonize an interaction. By such techniques, libraries approaching 1015 different nucleotide sequences have been generated in solution (see, for example, Bartel and Szostak (1993) Science 261:1411-1418; Bock et al. (1992) Nature 355:564; Ellington et al. (1992) Nature 355:850-852; and Oliphant et al. (1989) Mol Cell Biol 9:2944-2949).

According to one embodiment of the subject method, the SELEX (systematic evolution of ligands by exponential enrichment) is employed. See, for example, Tuerk et al. (1990) Science 249:505-510 for a review of SELEX. Briefly, in the first step of these experiments on a pool of variant nucleic acid sequences is created, e.g. as a random or semi-random library. In general, an invariant 3′ and (optionally) 5′ primer sequence are provided for use with PCR anchors or for permitting subcloning. The nucleic acid library is applied to screening a target specific binding pair, and nucleic acids which selectively bind (or otherwise act on the target) are isolated from the pool, the isolates are amplified by PCR and subcloned into, for example, phagemids. The phagemids are then transfected into bacterial cells, and individual isolates can be obtained and the sequence of the nucleic acid cloned from the screening pool can be determined.

When RNA is the test ligand, the RNA library can be directly synthesized by standard organic chemistry, or can be provided by in vitro translation as described by Tuerk et al., supra. Likewise, RNA isolated by binding to the screening target specific binding pair can be reverse transcribed and the resulting cDNA subcloned and sequenced as above.

iv) Small Molecule Libraries

Recent trends in the search for novel pharmacological agents have focused on the preparation of chemical libraries. Peptide, nucleic acid, and saccharide libraries are described above. However, the field of combinatorial chemistry has also provided large numbers of non-polymeric, small organic molecule libraries which can be employed in the subject method.

Exemplary combinatorial libraries include benzodiazepines, peptoids, biaryls and hydantoins. In general, the same techniques described above for the various formats of chemically synthesized peptide libraries are also used to generate and (optionally) encode synthetic non-peptide libraries.

B. Selecting Compounds from the Library

As with the diversity contemplated for the screening target and form in which the compound library is provided, the subject method is envisaged with a variety of detection methods for isolating and identifying compounds which agonize/antagonize an interaction. In most embodiments, the screening programs which test libraries of compounds will be derived for high throughput analysis in order to maximize the number of compounds surveyed in a given period of time. However, as a general rule, the screening portion of the subject method involves contacting the screening target specific binding pair with the compound library and isolating those compounds from the library which agonize/antagonize an interaction. The efficacy of the test compounds can be assessed by generating dose response curves from data obtained using various concentrations of the test compound. Moreover, a control assay can also be performed to provide a baseline for comparison.

Complex formation between a test compounds and a screening target specific binding pair may be directly detected by a variety of techniques. The complexes can be scored for using, for example, detectably labeled compounds, such as radiolabeled, fluorescently labeled, or enzymatically labeled polypeptides, by immunoassay, or by chromatographic detection.

In one embodiment, the variegated compound library is subjected to affinity enrichment in order to select for compounds which bind a preselected screening target specific binding pair. The term “affinity separation” or “affinity enrichment” includes, but is not limited to (1) affinity chromatography utilizing immobilizing screening targets, (2) precipitation using screening targets, (3) fluorescence activated cell sorting where the compound library is so amenable, (4) agglutination, and (5) plaque lifts. In each embodiment, the library of compounds are ultimately separated based on the ability of a particular compound to bind a screening target specific binding pair. See, for example, the Ladner et al. U.S. Pat. No. 5,223,409; the Kang et al. International Publication No. WO 92/18619; the Dower et al. International Publication No. WO 91/17271; the Winter et al. International Publication WO 92/20791; the Markland et al. International Publication No. WO 92/15679; the Breitling et al. International Publication WO 93/01288; the McCafferty et al. International Publication No. WO 92/01047; the Garrard et al. International Publication No. WO 92/09690; and the Ladner et al. International Publication No. WO 90/02809.

With respect to affinity chromatography, it will be generally understood by those skilled in the art that a great number of chromatography techniques can be adapted for use in the present invention, ranging from column chromatography to batch elution, and including ELISA and reverse biopanning techniques. Typically the screening target is immobilized on an insoluble carrier, such as sepharose or polyacrylamide beads, or, alternatively, the wells of a microtitre plate.

The population of compounds is applied to the affinity matrix under conditions compatible with the binding of compounds in the library to the immobilized screening target. The population is then fractionated by washing with a solute that does not greatly effect specific binding of compounds to the screening target, but which substantially disrupts any non-specific binding of components the library to the screening target or matrix. A certain degree of control can be exerted over the binding characteristics of the compounds recovered from the library by adjusting the conditions of the binding incubation and subsequent washing. The temperature, pH, ionic strength, divalent cation concentration, and the volume and duration of the washing can select for compounds within a particular range of affinity and specificity. Selection based on slow dissociation rate, which is usually predictive of high affinity, is a very practical route. This may be done either by continued incubation in the presence of a saturating amount of free screening target, or by increasing the volume, number, and length of the washes. In each case, the rebinding of dissociated compounds from the applied library is prevented, and with increasing time, compounds of higher and higher affinity are recovered. Moreover, additional modifications of the binding and washing procedures may be applied to find compounds with special characteristics. The affinities of some compounds may be dependent on ionic strength or cation concentration. Specific examples are peptides which depend on Ca++ or other ions for binding activity and which release from the screening target in the presence of a chelating agent such as EGTA. (see, Hopp et al. (1988) Biotechnology 6:1204-1210). Such peptides may be identified in the compound library by a double screening technique isolating first those that bind the screening target in the presence of Ca++, and by subsequently identifying those in this group that fail to bind in the presence of EGTA.

After “washing” to remove non-specifically members of the compound library, when desired, specifically compounds can be eluted by either specific desorption (using excess screening target) or non-specific desorption (using pH, polarity reducing agents, or chaotropic agents). In preferred embodiments using biological display packages, the elution protocol does not kill the organism used as the display package such that the enriched population of display packages can be further amplified by reproduction. The list of potential eluants includes salts (such as those in which one of the counter ions is Na+, NH4+, Rb+, SO42−, H2PO4−, citrate, K+, Li+, Cs+, HSO4−, CO32−, Ca2+, Sr2+, CL−, PO42−, HCO3−, Mg2+, Ba2+, Br−, HPO42−, or acetate), acid, heat, and, when available, soluble forms of the target antigen (or analogs thereof). Because bacteria continue to metabolize during the affinity separation step and are generally more susceptible to damage by harsh conditions, the choice of buffer components (especially eluates) can be more restricted when the display package is a bacteria rather than for phage or spores. Neutral solutes, such as ethanol, acetone, ether, or urea, are examples of other agents useful for eluting the bound display packages.

In preferred embodiments of biological peptide displays or certain nucleic acid libraries, affinity enriched packages or nucleic acids are iteratively amplified and subjected to further rounds of affinity separation until enrichment of the desired binding activity is detected. In certain embodiments, the specifically bound biological display packages, especially bacterial cells, need not be eluted per se, but rather, the matrix bound display packages can be used directly to inoculate a suitable growth media for amplification.

Where the display package is a phage particle, the fusion protein generated with the coat protein can interfere substantially with the subsequent amplification of eluted phage particles, particularly in embodiments wherein the cpIII protein is used as the display anchor. Even though present in only one of the 5-6 tail fibers, some peptide constructs because of their size and/or sequence, may cause severe defects in the infectivity of their carrier phage. This causes a loss of phage from the population during reinfection and amplification following each cycle of panning. In one embodiment, the peptide can be derived on the surface of the display package so as to be susceptible to proteolytic cleavage which severs the covalent linkage of at least the antigen binding sites of the displayed peptide from the remaining package. For instance, where the cpIII coat protein of M13 is employed, such a strategy can be used to obtain infectious phage by treatment with an enzyme which cleaves between the peptide portion and cpIII portion of a tail fiber fusion protein (e.g. such as the use of an enterokinase cleavage recognition sequence).

To further minimize problems associated with defective infectivity, DNA prepared from the eluted phage can be transformed into host cells by electroporation or well known chemical means. The cells are cultivated for a period of time sufficient for marker expression, and selection is applied as typically done for DNA transformation. The colonies are amplified, and phage harvested for a subsequent round(s) of panning.

After isolation of biological display packages which encode peptides having a desired binding specificity for the screening target, the nucleic acid encoding the peptide for each of the purified display packages can be recloned in a suitable eukaryotic or prokaryotic expression vector and transfected into an appropriate host for production of large amounts of protein.

On the other hand, where chemically synthesized libraries are used in the form of display packages, the isolated peptides are identified either directly from the display, e.g., by direct microsequencing, or the display packages are appropriately decoded, e.g., by elucidating the identity of an associated tag/index. Deconvolution techniques are also known in the art.

It will be apparent that, in addition to utilizing binding as the separation criteria, compound libraries can be fractionated based on other activities of the target molecule, such as modulation of catalytic activity.

4.8. Other Methods

In certain instances, it may be desirable to engineer stable mammalian cell lines expressing the Nub and Cub chimeric fusion polypeptides in order to facilitate screening applications of the invention. Methods for obtaining transgenic and knockout non-human animals are well known in the art. Knock out mice are generated by homologous integration of a “knock out” construct into a mouse embryonic stem cell chromosome which encodes the gene to be knocked out. In one embodiment, gene targeting, which is a method of using homologous recombination to modify an animal's genome, can be used to introduce changes into cultured embryonic stem cells. By targeting a Target gene of interest in ES cells, these changes can be introduced into the germlines of animals to generate chimeras. The gene targeting procedure is accomplished by introducing into tissue culture cells a DNA targeting construct that includes a segment homologous to a target Target gene locus, and which also includes an intended sequence modification to the Target genomic sequence (e.g., insertion, deletion, point mutation). The treated cells are then screened for accurate targeting to identify and isolate those which have been properly targeted.

Gene targeting in embryonic stem cells is in fact a scheme contemplated by the present invention as a means for disrupting a Target gene function through the use of a targeting transgene construct designed to undergo homologous recombination with one or more Target genomic sequences. The targeting construct can be arranged so that, upon recombination with an element of a Target gene, a positive selection marker is inserted into (or replaces) coding sequences of the gene. The inserted sequence functionally disrupts the Target gene, while also providing a positive selection trait. Exemplary Target gene targeting constructs are described in more detail below.

Generally, the embryonic stem cells (ES cells ) used to produce the knockout animals will be of the same species as the knockout animal to be generated. Thus for example, mouse embryonic stem cells will usually be used for generation of knockout mice.

Embryonic stem cells are generated and maintained using methods well known to the skilled artisan such as those described by Doetschman et al. (1985) J. Embryol. Exp. MoMFGFhol. 87:27-45). Any line of ES cells can be used, however, the line chosen is typically selected for the ability of the cells to integrate into and become part of the germ line of a developing embryo so as to create germ line transmission of the knockout construct. Thus, any ES cell line that is believed to have this capability is suitable for use herein. One mouse strain that is typically used for production of ES cells, is the 129J strain. Another ES cell line is murine cell line D3 (American Type Culture Collection, catalog no. CKL 1934) Still another preferred ES cell line is the WW6 cell line (Ioffe et al. (1995) PNAS 92:7357-7361). The cells are cultured and prepared for knockout construct insertion using methods well known to the skilled artisan, such as those set forth by Robertson in: Teratocarcinomas and Embryonic Stem Cells: A Practical Approach, E. J. Robertson, ed. IRL Press, Washington, D.C. [1987]); by Bradley et al. (1986) Current Topics in Devel. Biol. 20:357-371); and by Hogan et al. (Manipulating the Mouse Embryo: A Laboratory Manual, Cold Spring Harbor Laboratory Press, Cold Spring Harbor, N.Y. [1986]).

A knock out construct refers to a uniquely configured fragment of nucleic acid which is introduced into a stem cell line and allowed to recombine with the genome at the chromosomal locus of the gene of interest to be mutated. Thus a given knock out construct is specific for a given gene to be targeted for disruption. Nonetheless, many common elements exist among these constructs and these elements are well known in the art. A typical knock out construct contains nucleic acid fragments of not less than about 0.5 kb nor more than about 10.0 kb from both the 5′ and the 3′ ends of the genomic locus which encodes the gene to be mutated. These two fragments are separated by an intervening fragment of nucleic acid which encodes a positive selectable marker, such as the neomycin resistance gene (neo^{R}). The resulting nucleic acid fragment, consisting of a nucleic acid from the extreme 5′ end of the genomic locus linked to a nucleic acid encoding a positive selectable marker which is in turn linked to a nucleic acid from the extreme 3′ end of the genomic locus of interest, omits most of the coding sequence for Target gene or other gene of interest to be knocked out. When the resulting construct recombines homologously with the chromosome at this locus, it results in the loss of the omitted coding sequence, otherwise known as the structural gene, from the genomic locus. A stem cell in which such a rare homologous recombination event has taken place can be selected for by virtue of the stable integration into the genome of the nucleic acid of the gene encoding the positive selectable marker and subsequent selection for cells expressing this marker gene in the presence of an appropriate drug (neomycin in this example).

Variations on this basic technique also exist and are well known in the art. For example, a “knock-in” construct refers to the same basic arrangement of a nucleic acid encoding a 5′ genomic locus fragment linked to nucleic acid encoding a positive selectable marker which in turn is linked to a nucleic acid encoding a 3′ genomic locus fragment, but which differs in that none of the coding sequence is omitted and thus the 5′ and the 3′ genomic fragments used were initially contiguous before being disrupted by the introduction of the nucleic acid encoding the positive selectable marker gene. This “knock-in” type of construct is thus very useful for the construction of mutant transgenic animals when only a limited region of the genomic locus of the gene to be mutated, such as a single exon, is available for cloning and genetic manipulation. Alternatively, the “knock-in” construct can be used to specifically eliminate a single functional domain of the targetted gene, resulting in a transgenic animal which expresses a polypeptide of the targetted gene which is defective in one function, while retaining the function of other domains of the encoded polypeptide. This type of “knock-in” mutant frequently has the characteristic of a so-called “dominant negative” mutant because, especially in the case of proteins which homomultimerize, it can specifically block the action of (or “poison”) the polypeptide product of the wild-type gene from which it was derived. In a variation of the knock-in technique, a marker gene is integrated at the genomic locus of interest such that expression of the marker gene comes under the control of the transcriptional regulatory elements of the targeted gene. A marker gene is one that encodes an enzyme whose activity can be detected (e.g., b-galactosidase), the enzyme substrate can be added to the cells under suitable conditions, and the enzymatic activity can be analyzed. One skilled in the art will be familiar with other useful markers and the means for detecting their presence in a given cell. All such markers are contemplated as being included within the scope of the teaching of this invention.

As mentioned above, the homologous recombination of the above described “knock out” and “knock in” constructs is very rare and frequently such a construct inserts nonhomologously into a random region of the genome where it has no effect on the gene which has been targeted for deletion, and where it can potentially recombine so as to disrupt another gene which was otherwise not intended to be altered. Such nonhomologous recombination events can be selected against by modifying the abovementioned knock out and knock in constructs so that they are flanked by negative selectable markers at either end (particularly through the use of two allelic variants of the thymidine kinase gene, the polypeptide product of which can be selected against in expressing cell lines in an appropriate tissue culture medium well known in the art—i.e. one containing a drug such as 5-bromodeoxyuridine). Thus a preferred embodiment of such a knock out or knock in construct of the invention consist of a nucleic acid encoding a negative selectable marker linked to a nucleic acid encoding a 5′ end of a genomic locus linked to a nucleic acid of a positive selectable marker which in turn is linked to a nucleic acid encoding a 3′ end of the same genomic locus which in turn is linked to a second nucleic acid encoding a negative selectable marker Nonhomologous recombination between the resulting knock out construct and the genome will usually result in the stable integration of one or both of these negative selectable marker genes and hence cells which have undergone nonhomologous recombination can be selected against by growth in the appropriate selective media (e.g. media containing a drug such as 5-bromodeoxyuridine for example). Simultaneous selection for the positive selectable marker and against the negative selectable marker will result in a vast enrichment for clones in which the knock out construct has recombined homologously at the locus of the gene intended to be mutated. The presence of the predicted chromosomal alteration at the targeted gene locus in the resulting knock out stem cell line can be confirmed by means of Southern blot analytical techniques which are well known to those familiar in the art. Alternatively, PCR can be used.

Each knockout construct to be inserted into the cell must first be in the linear form. Therefore, if the knockout construct has been inserted into a vector (described infra), linearization is accomplished by digesting the DNA with a suitable restriction endonuclease selected to cut only within the vector sequence and not within the knockout construct sequence.

For insertion, the knockout construct is added to the ES cells under appropriate conditions for the insertion method chosen, as is known to the skilled artisan. For example, if the ES cells are to be electroporated, the ES cells and knockout construct DNA are exposed to an electric pulse using an electroporation machine and following the manufacturer's guidelines for use. After electroporation, the ES cells are typically allowed to recover under suitable incubation conditions. The cells are then screened for the presence of the knock out construct as explained above. Where more than one construct is to be introduced into the ES cell, each knockout construct can be introduced simultaneously or one at a time.

After suitable ES cells containing the knockout construct in the proper location have been identified by the selection techniques outlined above, the cells can be inserted into an embryo. Insertion may be accomplished in a variety of ways known to the skilled artisan, however a preferred method is by microinjection. For microinjection, about 10-30 cells are collected into a micropipet and injected into embryos that are at the proper stage of development to permit integration of the foreign ES cell containing the knockout construct into the developing embryo. For instance, the transformed ES cells can be microinjected into blastocytes. The suitable stage of development for the embryo used for insertion of ES cells is very species dependent, however for mice it is about 3.5 days. The embryos are obtained by perfusing the uterus of pregnant females. Suitable methods for accomplishing this are known to the skilled artisan, and are set forth by, e.g., Bradley et al. (supra).

While any embryo of the right stage of development is suitable for use, preferred embryos are male. In mice, the preferred embryos also have genes coding for a coat color that is different from the coat color encoded by the ES cell genes. In this way, the offspring can be screened easily for the presence of the knockout construct by looking for mosaic coat color (indicating that the ES cell was incorporated into the developing embryo). Thus, for example, if the ES cell line carries the genes for white fur, the embryo selected will carry genes for black or brown fur.

After the ES cell has been introduced into the embryo, the embryo may be implanted into the uterus of a pseudopregnant foster mother for gestation. While any foster mother may be used, the foster mother is typically selected for her ability to breed and reproduce well, and for her ability to care for the young. Such foster mothers are typically prepared by mating with vasectomized males of the same species. The stage of the pseudopregnant foster mother is important for successful implantation, and it is species dependent. For mice, this stage is about 2-3 days pseudopregnant.

Offspring that are born to the foster mother may be screened initially for mosaic coat color where the coat color selection strategy (as described above, and in the appended examples) has been employed. In addition, or as an alternative, DNA from tail tissue of the offspring may be screened for the presence of the knockout construct using Southern blots and/or PCR as described above. Offspring that appear to be mosaics may then be crossed to each other, if they are believed to carry the knockout construct in their germ line, in order to generate homozygous knockout animals. Homozygotes may be identified by Southern blotting of equivalent amounts of genomic DNA from mice that are the product of this cross, as well as mice that are known heterozygotes and wild type mice.

Other means of identifying and characterizing the knockout offspring are available. For example, Northern blots can be used to probe the mRNA for the presence or absence of transcripts encoding either the gene knocked out, the marker gene, or both. In addition, Western blots can be used to assess the level of expression of the MFGF gene knocked out in various tissues of the offspring by probing the Western blot with an antibody against the particular MFGF protein, or an antibody against the marker gene product, where this gene is expressed. Finally, in situ analysis (such as fixing the cells and labeling with antibody) and/or FACS (fluorescence activated cell sorting) analysis of various cells from the offspring can be conducted using suitable antibodies to look for the presence or absence of the knockout construct gene product.

Yet other methods of making knock-out or disruption transgenic animals are also generally known. See, for example, Manipulating the Mouse Embryo, (Cold Spring Harbor Laboratory Press, Cold Spring Harbor, N.Y., 1986). Recombinase dependent knockouts can also be generated, e.g. by homologous recombination to insert target sequences, such that tissue specific and/or temporal control of inactivation of a Target -gene can be controlled by recombinase sequences (described infra).

Animals containing more than one knockout construct and/or more than one transgene expression construct are prepared in any of several ways. The preferred manner of preparation is to generate a series of mammals, each containing one of the desired transgenic phenotypes. Such animals are bred together through a series of crosses, backcrosses and selections, to ultimately generate a single animal containing all desired knockout constructs and/or expression constructs, where the animal is otherwise congenic (genetically identical) to the wild type except for the presence of the knockout construct(s) and/or transgene(s).

A Target transgene can encode the wild-type form of the protein, or can encode homologs thereof, including both agonists and antagonists, as well as antisense constructs. In preferred embodiments, the expression of the transgene is restricted to specific subsets of cells, tissues or developmental stages utilizing, for example, cis-acting sequences that control expression in the desired pattern. In the present invention, such mosaic expression of a Target gene protein can be essential for many forms of lineage analysis and can additionally provide a means to assess the effects of, for example, lack of Target gene expression which might grossly alter development in small patches of tissue within an otherwise normal embryo. Toward this and, tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

Genetic techniques, which allow for the expression of transgenes can be regulated via site-specific genetic manipulation in vivo, are known to those skilled in the art. For instance, genetic systems are available which allow for the regulated expression of a recombinase that catalyzes the genetic recombination of a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of one of the subject Target gene proteins. For example, excision of a target sequence which interferes with the expression of a recombinant Target gene, such as one which encodes an antagonistic homolog or an antisense transcript, can be designed to activate expression of that gene. This interference with expression of the protein can result from a variety of mechanisms, such as spatial separation of the Target gene from the promoter element or an internal stop codon. Moreover, the transgene can be made wherein the coding sequence of the gene is flanked by recombinase recognition sequences and is initially transfected into cells in a 3′ to 5′ orientation with respect to the promoter element. In such an instance, inversion of the target sequence will reorient the subject gene by placing the 5′ end of the coding sequence in an orientation with respect to the promoter element which allow for promoter driven transcriptional activation.

The transgenic animals of the present invention all include within a plurality of their cells a transgene of the present invention, which transgene alters the phenotype of the “host cell” with respect to regulation of cell growth, death and/or differentiation. Since it is possible to produce transgenic organisms of the invention utilizing one or more of the transgene constructs described herein, a general description will be given of the production of transgenic organisms by referring generally to exogenous genetic material. This general description can be adapted by those skilled in the art in order to incorporate specific transgene sequences into organisms utilizing the methods and materials described below.

In an illustrative embodiment, either the cre/loxP recombinase system of bacteriophage P1 (Lakso et al. (1992) PNAS 89:6232-6236; Orban et al. (1992) PNAS 89:6861-6865) or the FLP recombinase system of Saccharomyces cerevisiae (O'Gorman et al. (1991) Science 251:1351-1355; PCT publication WO 92/15694) can be used to generate in vivo site-specific genetic recombination systems. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al. (1984) J. Biol. Chem. 259:1509-1514); catalyzing the excision of the target sequence when the loxP sequences are oriented as direct repeats and catalyzes inversion of the target sequence when loxP sequences are oriented as inverted repeats.

Accordingly, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation expression of a recombinant Target gene protein can be regulated via control of recombinase expression.

Use of the cre/loxP recombinase system to regulate expression of a recombinant Target gene protein requires the construction of a transgenic animal containing transgenes encoding both the Cre recombinase and the subject protein. Animals containing both the Cre recombinase and a recombinant Target gene can be provided through the construction of “double” transgenic animals. A convenient method for providing such animals is to mate two transgenic animals each containing a transgene, e.g., a Target gene and recombinase gene.

One advantage derived from initially constructing transgenic animals containing a Target transgene in a recombinase-mediated expressible format derives from the likelihood that the subject protein, whether agonistic or antagonistic, can be deleterious upon expression in the transgenic animal. In such an instance, a founder population, in which the subject transgene is silent in all tissues, can be propagated and maintained. Individuals of this founder population can be crossed with animals expressing the recombinase in, for example, one or more tissues and/or a desired temporal pattern. Thus, the creation of a founder population in which, for example, an antagonistic Target transgene is silent will allow the study of progeny from that founder in which disruption of Target gene mediated induction in a particular tissue or at certain developmental stages would result in, for example, a lethal phenotype.

Similar conditional transgenes can be provided using prokaryotic promoter sequences which require prokaryotic proteins to be simultaneous expressed in order to facilitate expression of the Target transgene. Exemplary promoters and the corresponding trans-activating prokaryotic proteins are given in U.S. Pat. No. 4,833,080.

Moreover, expression of the conditional transgenes can be induced by gene therapy-like methods wherein a gene encoding the trans-activating protein, e.g. a recombinase or a prokaryotic protein, is delivered to the tissue and caused to be expressed, such as in a cell-type specific manner. By this method, a Target A transgene could remain silent into adulthood until “turned on” by the introduction of the trans-activator.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used (Jackson Laboratory, Bar Harbor, Me.). Preferred strains are those with H-2b, H-2d or H-2q haplotypes such as C57BL/6 or DBA/1. The line(s) used to practice this invention may themselves be transgenics, and/or may be knockouts (i.e., obtained from animals which have one or more genes partially or completely suppressed).

In one embodiment, the transgene construct is introduced into a single stage embryo. The zygote is the best target for micro-injection. In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic animal will carry the incorporated transgene. This will in general also be reflected in the efficient transmission of the transgene to offspring of the founder since 50% of the germ cells will harbor the transgene.

Normally, fertilized embryos are incubated in suitable media until the pronuclei appear. At about this time, the nucleotide sequence comprising the transgene is introduced into the female or male pronucleus as described below. In some species such as mice, the male pronucleus is preferred. It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protamines of the male DNA with histones, thereby facilitating the combination of the female and male DNA complements to form the diploid zygote.

Thus, it is preferred that the exogenous genetic material be added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Introduction of the transgene nucleotide sequence into the embryo may be accomplished by any means known in the art such as, for example, microinjection, electroporation, or lipofection. Following introduction of the transgene nucleotide sequence into the embryo, the embryo may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. As regards the present invention, there will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present invention will include exogenous genetic material. As set out above, the exogenous genetic material will, in certain embodiments, be a DNA sequence which results in the production of a target protein (either agonistic or antagonistic), and antisense transcript, or a target mutant. Further, in such embodiments the sequence will be attached to a transcriptional control element, e.g., a promoter, which preferably allows the expression of the transgene product in a specific type of cell.

Retroviral infection can also be used to introduce transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

### 5. EXAMPLES

The present invention is further illustrated by the following examples which should not be construed as limiting in any way. The contents of all cited references (including literature references, issued patents, published patent applications as cited throughout this application} are hereby expressly incorporated by reference.

The practice of the present invention will employ, unless otherwise indicated, conventional techniques of cell biology, cell culture, molecular biology, microbiology and recombinant DNA, which are within the skill of the art. Such techniques are explained filly in the literature. See, for example, Molecular Cloning: A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); B. Perbal, A Practical Guide To Molecular Cloning (1984); the treatise, Methods In Enzymology (Academic Press, Inc., New. York.); Methods In Enzymology, Vols. 154 and 155 (Wu et al. eds.), Immunochemical Methods In Cell And Molecular Biology (Mayer and Walker, eds., Academic Press, London, 1987).

### Example 1

### Mapping the Molecular Environment of a Membrane Protein in vivo

The split-Ubiquitin (split-Ub) technique was used to map the molecular environment of a membrane protein in vivo. Cub, the C-terminal half of Ub, was attached to Sec63p, and Nub, the N-terminal half of Ub, was attached to a selection of differently localized proteins of the yeast Saccharomyces cerevisiae. The efficiency of the Nub and Cub reassembly to the quasi-native Ub reflects the proximity between Sec63-Cub and the Nub-labeled proteins. By using a modified Ura3p as the reporter that is released from Cub, the local concentration between Sec63-Cub-RUra3p and the different Nub-constructs could be translated into the growth rate of yeast cells on media lacking uracil. We show that Sec63p interacts with Sec62p and Sec61p in vivo. Ssh1p is more distant to Sec63p than its close sequence homologue Sec61p. Employing Nub- and Cub-labeled versions of Ste14p, an enzyme of the protein isoprenylation pathway, we conclude that Ste14p is a membrane protein of the ER. Using Sec63p as a reference, a gradient of local concentrations of different t- and v-SNARES could be visualized in the living cell. The RUra3p reporter should further allow the selection of new binding partners of Sec63p and the selection of molecules or cellular conditions that interfere with the binding between Sec63p and one of its known partners.

Construction of Test Proteins

The Cub-RUra3 reporter module was constructed by PCR amplification. The fragment covered residues 35-76 of UBI4 and a SalI and BamHI site to bring the fragment in front of the LACI-URA3 gene fusion (Ghislain et al., 1996). The sequence between the C terminus of Cub and the LACI sequence of the RURA3 reads: GGT GGT AGG CAC GGA TCC. The last two residues of the Cub and the N-terminal arginine of the RURA3 are printed in bold letters; the BamHI site is underlined. SEC63-Cub-RURA3 was constructed by PCR amplification of the last 445 base pairs (bp) of the coding sequence of SEC63 not including the stop codon by using genomic DNA of S. cerevisiae as a template. The ends of the PCR product contained restriction sites to allow the in-frame fusion with the Cub-RURA3 module located in the vector pRS305 (Sikorski and Hieter, 1989). The short linker sequence between the last codon of SEC63 and the first codon of Cub reads: GAA GGC GGG TCG ACC GGT. The last codon of SEC63 and the first codon of Cub are in bold letters; the SalI site is underlined. The vector was cut at its unique PstI site in the SEC63 -containing fragment and transformed into the S. cerevisiae strains JD51 and JD55 to yield, through homologous recombination, the integrated cassette that expressed Sec63-Cub-RUra3p from the native promoter of SEC63 and a short C-terminal fragment of SEC63 comprising its last 448 bp. Integration was confirmed by PCR. SEC63-Cub-Dha was created in a similar manner. The linker between SEC63 and the Cub-Dha module reads: GAA GGC GGG TCG ACC ATG TCG GGG GGG. The last codon of SEC63 and the first codon of Cub are printed in bold letters. The Cub-Dha module is described by Johnsson and Varshavsky (1994). FUR4-Cub-RURA3 was created similar to SEC63-Cub-RURA3. The PCR product containing the last 952 bp of the ORF of the FUR4 gene were inserted in front of the Cub-RURA3 module located in the pRS303 vector using an EagI and a SalI site at the ends of the PCR product. The linker between the last codon (bold letters) of FUR4 and the first codon of Cub (bold letters) reads: ATT GGG TCG ACC GGT. The SalI site is underlined. The vector was cut at the unique EcoRI site in the FUR4-derived fragment to create, through homologous recombination, a C-terminal fragment of the gene of 955 bp and the integrated cassette that expressed Fur4-Cub-RUra3p from the FUR4 promoter. Integration was confirmed by PCR. Two nucleotide exchanges were found in the FUR4 PCR product when compared with the corresponding sequence in the yeast genome database leading to an Asp and Glu in position 421 and 617 of the Fur4p-construct instead of the Asn and Val encoded in the genomic sequence. Since Fur4p-Cub-RUra3p still conferred 5-fluoroorotic acid (5-FOA) sensitivity to the transformed yeast, we inferred that the Cub construct is functional. STE14-Cub-RURA3 was constructed using two primers to amplify the complete ORF of STE14 using genomic DNA as a template. The PCR product was inserted between the Cub-RURA3 module and the P_{MET25}-promoter in the vector pRS315. The linker between the last codon (bold letters) of STE14 and the first codon of Cub (bold letters) reads: ATA GGG TCG ACC GGT. The SalI site is underlined. The same PCR product was inserted between the P_{GAL1}-promoter and Dha to create STE14-Dha in the pRS314 vector. The sequence between the last codon of STE14 and Dha reads: ATA GGG TCG ACC TTA ATG CAG AGA TCT GGC ATC ATG GTT. The last codon of STE14 and the first two codons of Dha are underlined. The sequence connecting the last codon of SEC62 (underlined) and Dha of SEC62-Dha in pRS314 reads: AAC GGC GGG TCG ACC TTA ATG CAG AGA TCT GGC ATC ATG GTT. TOM20-Cub-RURA3 was constructed similar to STE14-Cub-RURA3. The PCR product was inserted between the PCUP1-promoter and the Cub-RURA3 module in the vector pRS315. The linker between the last codon of TOM20 (bold letters) and the first codon of Cub (bold letters) reads: GAC GGG TCG ACC GGT. The SalI site is underlined.

The Nub-constructs were assembled from the P_{CUP1}-Nub-cassette and a PCR fragment containing the ORF or part of the ORF of the desired gene to finally reside in the vector pRS314, pRS313, or pRS304. A BamHI site was used to bring the Nub in frame with the PCR product. The linker between the last codon of Nub (bold letters) and the first codon of the following ORF (bold letters) reads: GG ATCCCT GGC GTC for TOM22, GG ATCCCT GGG TCT GGG ATG for SEC61 and SSH1, GG ATC CCT GGG GAT ATG for SNC1, SSO1, TPI1, GUK1, GG ATC CCT GGG GAT TCC for VAM3. The BamHI site is underlined. Nub-SEC61 was constructed by targeted integration of a Nub-SEC61-containing fragment into SEC61 of the S. cerevisiae strain JD53. A fragment containing the first 875 bp of the SEC61 ORF was amplified by PCR and inserted downstream of the pRS304- or pRS303-based P_{CUP1}-Nub cassette, using the flanking BamHI and EcoRI sites. For targeted integration, the plasmid was linearized at the unique StuI site in the SEC61 ORF to create the yeasts NJY61-I, -A, and -G. Integration was confirmed by PCR. To construct Nub-Ssh1p, a fragment of 680 bp was amplified by PCR and inserted downstream of the pRS304-based P_{CUP1}-Nub cassette using the flanking BamHI and XhoI sites. The vector was cut for targeted integration at the unique ClaI site in the SSH1 ORF to create the yeast strains NJY78-1, -A, -G, and -VI. Integration was confirmed by PCR. The construction of Nub-SEC62, -SED5, -STE14, and -BOS1 was described in Dünnwald et al. (Mol. Biol. Cell 10: 329-344, 1999). The functionality of Nub-Sed5p and -Sec62p was confirmed by complementing a yeast strain carrying a ts mutation in the corresponding gene. Nub-Sso1p, Nub-Guk1p, and Nub-Tpi1p were shown to support growth of S. cerevisiae cells under conditions where the corresponding, unmodified protein was not expressed. Nub-Snc1p, -Tom22p, -Vam3p, and -Ssh1p were not tested. The functionality of Nub-Sec61p in the strain NJY61-I was tested by repeating the transformation of JD53 with a StuI cut vector bearing a shift in the reading frame between Nub and SEC61. As a consequence, no full-length Sec61p should be expressed in the transformed haploids, but only the N-terminal fragment from the first 875 bp of the SEC61 ORF. Viable haploids would document that the N-terminal fragment of Sec61p can substitute for the full-length protein. However, the occasional colonies that were obtained after transformation were shown by PCR to always harbor a native SEC61 in addition to the modified Nub-SEC61 allele carrying the frame shift between the Nub and the SEC61 ORF. This shows that in the strain NJY61-I, the essential function of Sec61p was contributed by Nub-Sec61p.

Assays

Immunoblotting

Cell extraction for immunoblotting was performed essentially as described (Johnsson and Varshavsky, 1994). Proteins were fractionated by SDS-12.5% PAGE and electroblotted on nitrocellulose membranes (Schleicher & Schuell, Dassel, Germany), using a semidry transfer system (Hoeffer Pharmacia Biotech, San Francisco, Calif.). Blots were incubated with a monoclonal anti-ha antibody (Babco, Richmond, Calif.), and bound antibody was visualized using horseradish peroxidase-coupled rabbit anti-mouse antibody (Bio-Rad, Hercules, Calif.), the chemiluminescence detection system (Boehringer, Mannheim, Germany), and x-ray films (Kodak, Rochester, N.Y.).

Growth Assay and Mating Assay

Yeast-rich (YPD) and synthetic minimal media with 2% dextrose (SD) or 2% galactose (SG) were prepared as described (Dohmen et al., 1995). S. cerevisiae cells were grown at 30° C. in liquid selective media containing uracil. Cells were diluted in water and 4 μl were spotted on agar plates, selecting for the presence of the fusion constructs but lacking uracil or containing 1 mg/ml 5-FOA (WAK-Chemie, Bad Soden, Germany) and 50 μg/ml uracil. The same dilutions were spotted on plates containing uracil to check for cell numbers. The plates were incubated at 30° C. for 3-5 d unless stated otherwise. Mating tests were performed as described (Michaelis and Herskowitz, 1988).

Deletion of STE14

The open reading frame of STE14 was replaced by the dominant kan^{r} marker essentially as described by Güildener et al. (1996). The PCR primers used for the construction of the kan^{r} disruption cassette were 5′-CCCCCTCTTTCATTGTGGTCACCGTTTTTGAAC ACAACCAGCTGAAGCTTCGTACGC and 5′-CACAAAAATCCAGTCCATAACTAACACAATCATTACTAGCATAGGCCACTAGGTGATCTG. Underlined are the sequences immediately preceding the ATG or following the stop codon of the coding sequence of STE14 (Sapperstein et al., 1994). Transformed yeast cells were selected for kan^{r} integration by Geneticin (Life Technologies, Paisley, Scotland), and the deletion was verified by diagnostic PCR and the mating deficiency of the cells.

Experimental Results

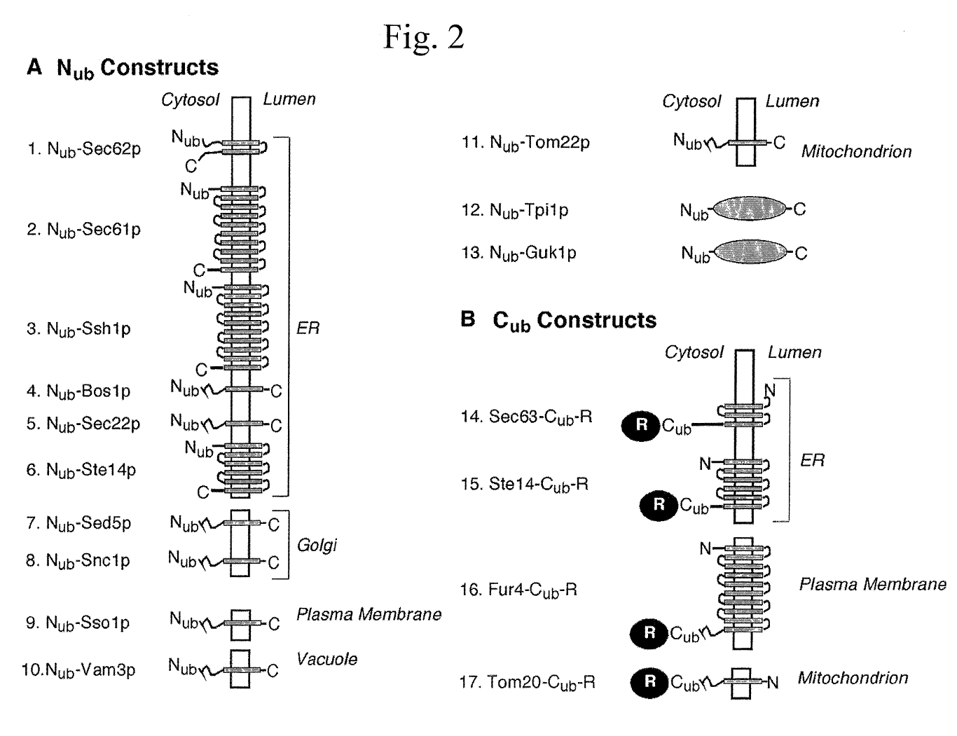

Sec63p was extended at its C-terminus with Cub that was linked to an N-terminally modified version of the enzyme Ura3p (RUra3p) to create Sec63-Cub-RUra3p (Sec63CRUp) (FIGS. 1 and 2). Due to the topology of Sec63p, CRUp points into the cytosol of the cell (Feldheim et al., 1992). By coexpressing a set of Nub-fusion proteins (Nub-X in FIG. 1), we first attempted to distinguish between Sec63p-interacting and -noninteracting proteins.

FIG. 1 depicts the split-Ubiquitin technique and its application to the analysis of membrane proteins using a metabolic marker. Cub-RUra3p was linked to the C terminus of Sec63p, and Nub was linked to the N terminus of the membrane protein P1. Pathway 1: Nub is coupled to a protein that binds to Sec63p. The complex brings Nub and Cub into close proximity. Nub and Cub reconstitute the quasi-native Ub that is cleaved by the Ub-specific proteases to release RUra3p from Cub. The cleaved RUra3p is targeted for rapid destruction by the enzymes of the N-end rule (3) to yield cells that are uracil auxotrophs and 5-FOA resistant. Pathway 2: Nub is linked to a protein that does not bind to Sec63p. The two fusion proteins do not improve the reconstitution of Nub and Cub into the quasi-native Ub. Thus, RUra3p stays linked to Sec63-Cub, and the cells are uracil prototrophs and 5-FOA sensitive.

In pathway 1, P1 is a protein that strongly interacts with Sec63p. Nub and Cub reassemble to the quasi-native Ub, and RUra3p is cleaved by the UBPs. Since the N-terminal residue of the released RUra3p is an arginine, rapid degradation of RUra3p by the enzymes of the N-end rule ensures that the cells stop dividing on plates lacking uracil (Ura). 5-FOA is converted by Ura3p into 5-fluorouracil, which is toxic for the cell. Therefore the rapid degradation of RUra3p due to the interaction between protein P1 and Sec63p allows the cells to grow on plates containing 5-FOA (FOAR) (Ghislain et al., 1996; Johnsson and Varshavsky, 1997; Varshavsky, 1997). Pathway 2: P1 is a protein that does not interact with Sec63p. The linked Nub and Cub do not or only partially reassemble to the quasi-native Ub. The cells retain sufficient unclipped Sec63CRUp to stay Ura+ and 5-FOA-sensitive (FOAS). As an alternative to the RUra3p reporter, Sec63p-Cub was extended by the enzyme dihydrofolate reductase that carries an ha tag at its C terminus (Sec63-Cub-Dha). The cleaved Dha remains stable in the cytosol and can be detected together with the unclipped fusion protein by immunoblotting with antibodies directed against the ha epitope (Johnsson and Varshavsky, 1994).

Monitoring the Interaction between Sec62p and Sec63p In Vivo

Sec63CRUp and Sec63-Cub-Dha were integrated into diploid cells via homologous recombination to replace one native copy of Sec63p. Tetrad analysis of the sporulated diploids validated that both Sec63-Cub-fusion proteins are functional (our unpublished observation). Since the two spores containing the modified versions of Sec63p grew slightly slower, the interaction assay was performed in diploid cells. To test the interaction between Sec62p and Sec63p, the Nub-moiety was linked to the cytosolic N-terminus of Sec62p (FIG. 2).

FIG. 2 depicts the Nub and Cub fusions utilized. (A) Nub (residues 1-36 of Ub) was fused to the N terminus of either a transmembrane protein (constructs 1-11) or a cytosolic protein (constructs 12-13). The N termini of all proteins are located in the cytosol. The orientation and the numbers of the membrane-spanning domains were obtained from published studies. The orientation of the N and the C terminus of Ste14p and its subcellular localization was a subject of this study. The Nub-attached proteins of constructs 1-5 are localized in the ER (Deshaies and Schekman, 1990; Shim et al., 1991; Finke et al., 1996 ; Wilkinson et al., 1996 ; Ballensiefen et al., 1998 ). The localization of the Nub-attached protein of construct 6 was a subject of this study. The Nub-attached protein of construct 7 resides in the early Go1gi and of construct 8 in the late Golgi/plasma membrane (Protopopov et al., 1993; Banfield et al., 1994). The Nub-attached protein of construct 9 was shown to be in the plasma membrane (Aalto et al., 1993 ). The Nub-attached protein of construct 10 was found in the vacuole, and the Nub-attached protein of construct 11 was found in the outer membrane of the mitochondrion (Kiebler et al., 1993; Darsow et al., 1997; Wada et al., 1997; Srivastava and Jones, 1998). (B) Cub (residues 35-76 of Ub) was linked to the C terminus of a transmembrane protein and extended at its own C terminus by a reporter protein. The C termini of all proteins are localized in the cytosol. The information on the orientation of the N- and C-termini, the numbers of the membrane-spanning domains, and the localization of the unmodified proteins were obtained from published studies except for construct 15, where the number of membrane-spanning domains is still tentative. The Cub-attached protein of construct 14 is localized in the ER, that of construct 16 is found in the plasma membrane, and that of construct 17 is localized in the outer membrane of the mitochondrion (Jund et al., 1988 ; Feldheim et al., 1992; Moczko et al., 1997 ). The reporter (R) is RUra3p for the constructs 15-17 and RUra3p or DHFRha (Dha) for construct 14.

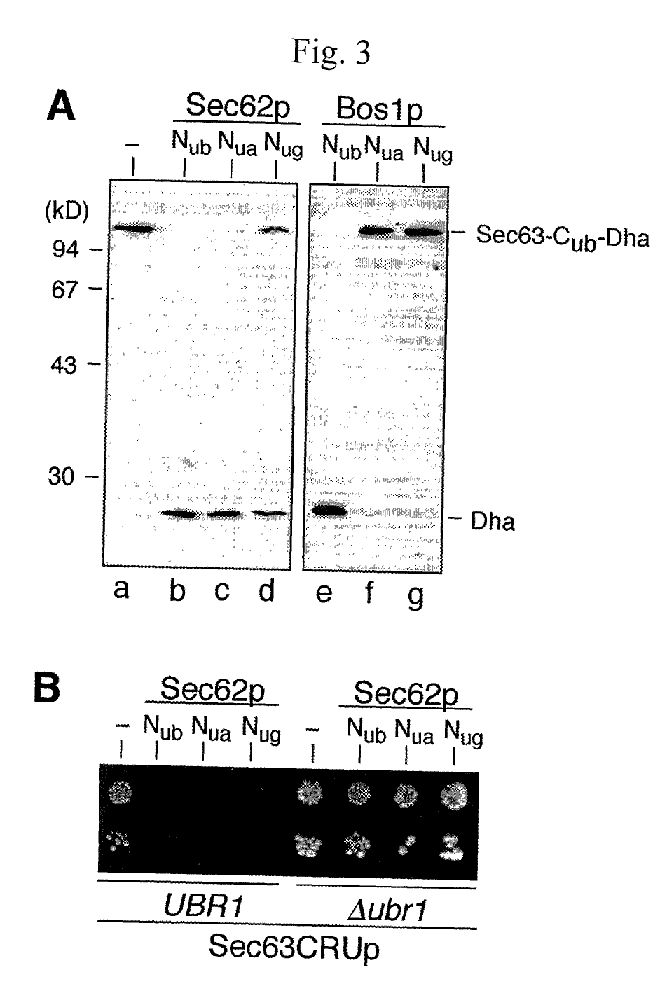

The Nub-Sec62p is functional (Dünnwald et al., 1999). Immunoblot analysis of protein extracts from cells expressing Sec63-Cub-Dha together with Nub- or Nua-Sec62p showed that Sec63-Cub-Dha is completely converted into Sec63-Cub and Dha. Nug-Sec62p still induces more than 60% cleavage (FIG. 3A). The ratio of cleaved to uncleaved Cub-Dha matches the ratio seen for the interaction between two correspondingly labeled Nub- and Cub-zipper proteins, reinforcing the interpretation of a tight interaction between Sec62p and Sec63p (Johnsson and Varshavsky, 1994). Bos1p, a membrane protein of the ER that does not interact with Sec63p, induces significant cleavage of Sec63-Cub-Dha when labeled with Nub, but hardly induces any cleavage when labeled with Nua or Nug (FIGS. 2 and 3A).

FIG. 3 depicts the use of the split-Ub method to monitor the interaction between Sec63p and Sec62p in vivo. (A) Immunoblot analysis of cells expressing Sec63-Cub-Dha together with an empty plasmid (lane a) or together with Nub-, Nua-, or Nug-Sec62p (lanes b, c, and d, respectively) or Nub-, Nua-, or Nug-Bos1p (lanes e, f, and g, respectively). The nitrocellulose membrane was probed with the anti-ha antibody that recognizes the uncleaved Cub fusion and the cleaved Dha. (B) Growth assay of the interaction between Sec63p and Sec62p based on split-Ub and a short-lived Ura3p (RUra3p) as a reporter. Sec63CRUp-containing cells bearing either the UBR1 gene or a UBR1 deletion were transformed with an empty plasmid or Nub-, Nua-, or Nug-Sec62p. Cells were pregrown in selective media containing uracil. Cells (103 or 102) were spotted on selective plates lacking uracil and also lacking leucine and tryptophan to select for the presence of the Cub- and Nub-constructs.

Cells harboring Sec63CRUp grow on medium lacking uracil. The same cells coexpressing Nub-, Nua- or Nug-Sec62p grow on medium containing uracil but fail to grow on medium lacking uracil (FIG. 3B). To test whether this new phenotype of the Sec63CRUp containing cells is due to the ability of Nub-Sec62p to induce cleavage and the rapid degradation of RUra3p, we expressed the same Nub/Cub combination in congenic yeast cells harboring a deletion of UBR1 (FIG. 3B). UBR1 encodes the recognition component of the N-end rule pathway, and proteins bearing destabilizing N-terminal residues that are rapidly degraded in wild-type cells are stabilized in ubr1 cells (Bartel et al., 1990). Since ubr1 cells carrying Nub-Sec62p and Sec63CRUp are still Ura+, we conclude that in wild-type cells bearing Sec63CRUp, Nub-Sec62p causes the cleavage and degradation of RUra3p.

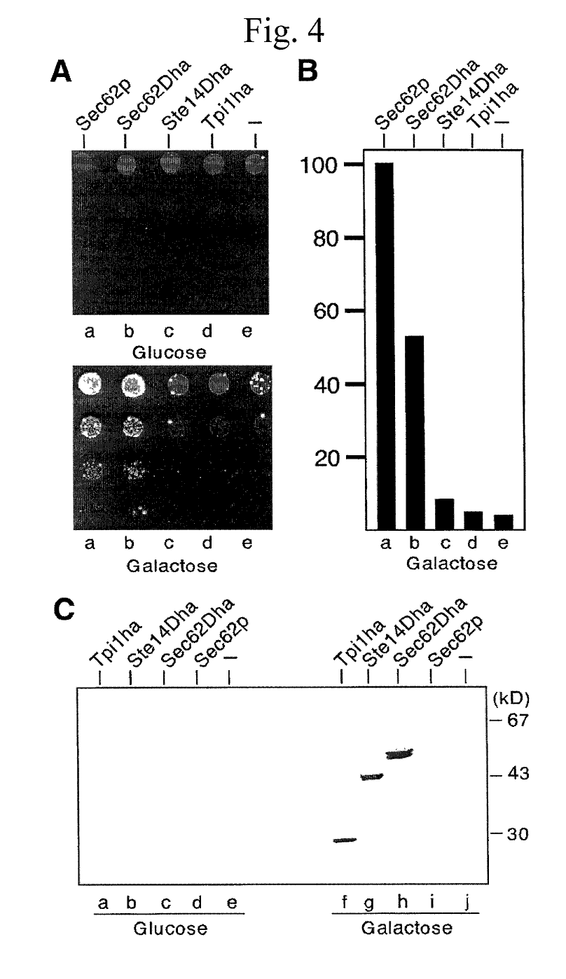

The measured proximity between Nub-Sec62p and Sec63CRUp is a strong indicator, albeit not proof, that Sec63p and Sec62p are components of one protein complex. If the efficient reassociation of Nug-Sec62p and Sec63CRUp is a consequence of a direct protein interaction, overexpression of the unlabeled Sec62p should displace its Nub-labeled counterpart in the complex. As a consequence, the local concentration between Nub-Sec62p and Sec63CRUp will decrease, less RUra3p will be cleaved, and the cells will start to grow on plates lacking uracil. We expressed the unmodified Sec62p and a Sec62p derivative that carries the Dha extension at its C terminus (Sec62-Dha) from the inducible P_{GAL1}-promoter in the presence of Nug-Sec62p and Sec63CRUp. The triply transformed cells were spotted on plates lacking uracil that either contained glucose to repress or contained galactose to induce the expression of Sec62p or Sec62-Dha. The growth of the cells on plates that lacked uracil but contained galactose confirmed the displacement of Nug-Sec62p by Sec62p or Sec62-Dha (FIG. 4A). To verify the specificity of this experiment, the competition was repeated with the membrane protein Ste14p and the cytosolic Triose phosphate isomerase (Tpi1p) that were expressed from the P_{GAL1}-promoter and C-terminally extended by the Dha module (Ste14-Dha) or the ha-epitope (Tpi1-ha). Dha and ha served in these constructs as a tag to allow the immunodetection of the correspondingly labeled proteins. In contrast to the expression of Sec62p or Sec62-Dha, the overexpression of Ste14-Dha and Tpi1-ha had no effect on the growth of the cells harboring Sec63CRUp and Nug-Sec62p (FIG. 4A). Immunoblots confirmed the expression of all ha-bearing proteins (FIG. 4C), and a Sec62p-specific antibody confirmed the expression of the P_{GAL1}-driven Sec62p (our unpublished observation). Using the Sec62p-specific antibody, we could also demonstrate that the expression of Nug-Sec62p was not influenced by galactose (our unpublished observation). To semiquantitatively measure the influence of Sec62p overexpression on the interaction between Nug-Sec62p and Sec63CRUp, roughly 10,000 cells were plated on galactose-containing mediun without uracil, and the yeast colonies were counted after 4 d (FIG. 4B). Approximately 800 colonies were recovered upon overexpression of Sec62p, and 400 colonies were recovered upon overexpression of Sec62-Dha, suggesting that the extension at the C terminus of Sec62p might already interfere with the ability of the molecule to interact with Sec63p. Around 30 colonies were recovered from yeast cells carrying the empty P_{GAL1}-promoter, and an average of 60 and 40 colonies were recovered upon coexpression of Ste14-Dha and Tpi1-Dha. The competition of Nug-Sec62p by Sec62p shows that the split-Ub measured proximity between Sec62p and Sec63p is a consequence of both proteins being components of one protein complex.

FIG. 4 demonstrates that the measured proximity between Sec62p and Sec63p is due to both proteins being in one complex. (A) Cells bearing Sec63CRUp and Nug-Sec62p were transformed with a plasmid containing either Sec62p, Sec62Dha, Ste14Dha, Tpi1ha, or an empty plasmid, all under the control of the P_{GAL1}-promoter (lanes a-e). Approximately 105, 104, 103, and 102 cells were spotted on selective media lacking uracil and containing either glucose to repress or galactose to induce the P_{GAL1} promoter. (B) S. cerevisiae cells (104) were plated as described in panel A on selective media containing galactose and lacking uracil, and colonies were counted after 4 d. The average of seven independent experiments is shown. Approximately 800 colonies were recovered upon overexpression of Sec62p. This number was arbitrarily set as 100. (C) Overexpression of the ha epitope-bearing proteins was confirmed by immunoblot analysis of extracts of S. cerevisiae cells coexpressing Sec63CRUp, Nug-Sec62p, and the following constructs: Tpi1ha (lanes a and f), Ste14Dha (lanes b and g), Sec62Dha (lanes c and h), Sec62p (lanes d and i), and empty vector (lanes e and j). Cells were grown in glucose (lanes a-e) to repress and grown in galactose (lanes f-j) to induce the expression of the proteins.

Monitoring the Distance of the Unlabeled Protein to Sec 63p

Every protein displays a characteristic spectrum of local concentrations toward the other proteins inside the cell. Split-Ub allows comparison of the local concentrations that exist between different Nub-labeled proteins and a common Cub-fusion. The proteins of high local concentration will need a Nub with a lower affinity to Cub to achieve Nub-Cub reassembly than the proteins of low local concentration. The RUra3p reporter will translate these differences into the growth rate of the yeasts. Cells harboring a Nub-labeled protein that is close to a CRUp-fusion do not grow or grow slower than cells carrying a Nub-labeled protein that is more distant. We started to map the spectrum of local concentrations of Sec63p by comparing the interactions of Sec63CRUp with 13 different Nub-, Nua-, and Nug fusions. The proteins were chosen to cover a wide range of local concentrations by predominantly selecting membrane proteins, whose distances to Sec63p are adjusted by their distinct distribution in the cell. Sec61p as a member of the heptameric Sec complex should be very close, whereas Tom22p as a membrane protein of the outer mitochondrial membrane should be very distant to Sec63p. The topology of all Nub-modified proteins and the cellular localization of the unmodified proteins are shown in FIG. 2. Since the local concentration of two proteins is influenced by their amount and their cellular distribution, we tried to minimize the differences in total amount by expressing all Nub-fusions from the noninduced P_{CUP1}-promotor.

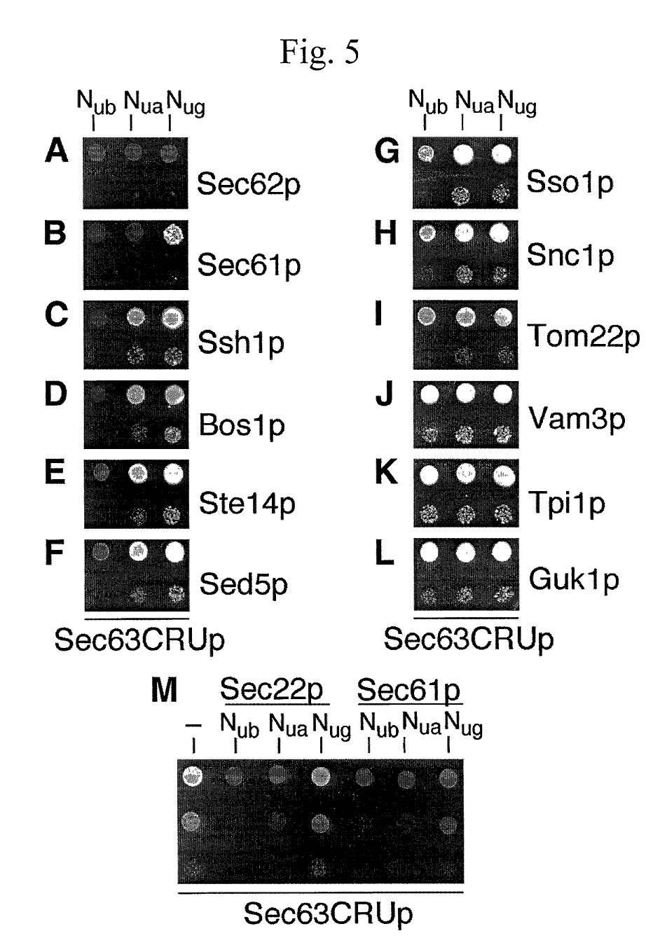

FIG. 5 shows the use of the split-Ub technique to measure the proximity between Sec63p and membrane-associated proteins in vivo. Sec63CRUp containing cells expressing Nub, Nua, and Nug constructs of Sec62p (A), Sec61p (B), Ssh1p (C), Bos1p (D), Ste14p (E), Sed5p (F), Sso1p (G), Snc1p (H), Tom22p(I), Vam3p (J), Tpi1p (K), and Guk1p (L) were spotted (105 and 103 cells) on selective media lacking uracil (A-M) and leucine and histidine (A and D) or leucine and tryptophan (B, C, and E-M) to select for the presence of the Cub and Nub constructs. (M) Sec63CRUp-containing cells bearing either the empty plasmid, Nub-, Nua-, -Nug-Sec22p or Nub-, Nua-, Nug-Sec61p were spotted (10⁵, 10⁴, 10³ cells) on plates lacking uracil. Cells were grown for 4 d.

The different growth of the transformed cells on SD-ura allows us to clearly separate the Nub constructs of the two known Sec63p-interacting proteins, Sec62p and Sec61p, from all the other Nub constructs (FIG. 5 and Table 1). The Nub and Nua constructs of both proteins completely inhibit the growth of the Sec63CRUp-bearing cells. The Nug construct inhibits growth in the case of Sec62p and strongly impairs growth in the case of Sec61p. Sec63CRUp-containing cells transformed with any other Nug construct show unimpaired growth on media lacking uracil. Furthermore, the assay allows us to distinguish between the Nub constructs of those proteins that do not bind to Sec63p (FIG. 5 and Table 1). According to the growth of the transformed yeasts, we could arrange the Nub constructs into five groups of decreasing proximity to Sec63p. The classification approximately reflects the localization of the unlabeled proteins (see FIG. 1 and Table 1). Groups 1 and 2 comprise the Sec63p-binding proteins Sec62p and Sec61p.

TABLE 1
Growth of cells containing Sec63CRUp and different
Nub constructs

| Protein Nub | | Nua | Nug | FOA | Group |
|---|---|---|---|---|---|
| Sec62p | | | | R | 1 |
| Sec61p | | | + | R | 2 |
| Sec22p | | (+) | +++ | R | 3 |
| Ssh1p | | ++ | +++ | S | 3 |
| Bos1p | | ++ | +++ | S | 3 |
| Ste14p | | ++ | +++ | S | 3 |
| Sed5p | (+) | ++ | +++ | S | 3 |
| Sso1p | + | +++ | +++ | S | 4 |
| Snc1p | + | +++ | +++ | S | 4 |
| Tom22p | + | ++ | +++ | ND | 4 |
| Vam3p | +++ | +++ | +++ | S | 5 |
| Tpi1p | +++ | +++ | +++ | S | 5 |
| Guk1p | +++ | +++ | +++ | S | 5 |

Growth was scored on plates lacking uracil. The number of pluses denotes the robustness of the growth of the colonies. The column FOA indicates the behavior of the corresponding Nua construct-bearing cells on plates containing 5-FOA. R, the cells are 5-FOA resistant and grow; S, the cells are 5-FOA sensitive.

Group 3 includes the proteins whose Nub constructs abolish the growth of Sec63CRUp cells, whose Nua constructs inhibit their growth to varying degrees but whose Nug constructs allow full growth on media lacking uracil (FIG. 5 and Table 1). Group 3 includes the proteins Ssh1p, Bos1p, Ste14p, Sec22p, and Sed5p (FIG. 5 and Table 1). Sec22p, Bos1p, and Ssh1p localize in the ER, whereas Sed5p resides in the early Go1gi, the compartment that is functionally adjacent to the ER (Shim et al., 1991; Hardwick and Pelham, 1992; Banfield et al., 1994; Finke et al., 1996; Ballensiefen et al., 1998).

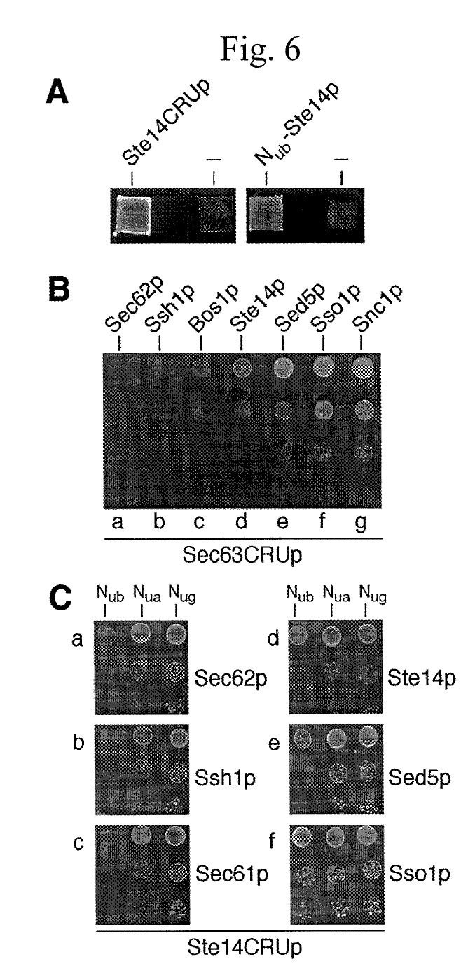

FIG. 6 shows: (A) Nub and Cub constructs of Ste14p are functional. Nub-Ste14p and Ste14CRUp were expressed in cells containing a STE14 deletion and mated with an appropriate tester strain of the opposite mating type. The mated cells were patched on media selecting for the formation of diploids. (B) Ste14p is located between Bos1p and Sed5p. Sec63CRUp containing cells expressing Nvi-Sec62p (a),-Ssh1p (b),-Bos1p (c),-Ste14p (d),-Sed5p (e),-Sso1p (f), and -Snc1p (g) were spotted (105, 104, 103, and 102 cells) on SD-ura plates that also lacked leucine and tryptophan to select for the presence of the Cub and Nvi constructs. Cells were grown for 3 d. (C) Sec62p, Ssh1p, and Sec61p are equidistant to Ste14p. Ste14CRUp-containing cells expressing Nub, Nua, and Nug constructs of Sec62p (a), Ssh1p (b), Sec61p (c), Ste14p (d), Sed5p (e), and Sso1p (f) were spotted (10⁵, 10³, and 10² cells) on selective media lacking uracil, leucine, and tryptophan and containing 500 μM methionine to reduce the expression of Ste14CRUp. Cells were grown for 3 days.

In contrast to all the other analyzed proteins, the localization and topology of Ste14p were unknown when we started its analysis. STE14 encodes an enzyme that methylates the C terminus of the CAAX box motif-containing proteins such as the small GTPases, Ras1p, Cdc42p, or Rho1p (Sapperstein et al., 1994; Zhang and Casey, 1996). The corresponding activity in mammalian cells was shown to be associated with a microsomal membrane fraction (Stephenson and Clarke, 1990). Functionality of Nub-Ste14p was confirmed by complementing the mating defect of a STE14 deletion strain (FIG. 6A). Nub-Ste14p induces the cleavage of Cubs that are localized in the cytosol, implying that the N terminus of the protein is in the cytosol of the cell (FIG. 5; Dünnwald et al., 1999). Since the interaction between Nub-Ste14p and Sec63CRUp is comparable to the interactions of the correspondingly labeled Bos1p, Ssh1p, and Sed5p, Ste14p might be localized in the ER, the Go1gi, or in both compartments. To better resolve the localization of Ste14p, we had to search for a Nub mutant whose affinity to Cub falls between the affinities of wild-type Nub and Nua. This was accomplished by exchanging isoleucine 3 of Nub against a valine (Nvi) (Eckert, Raquet, and Johnsson, unpublished observation). FIG. 6B shows the growth of the Sec63CRUp-containing cells transformed with Nvi-Sec62p, -Ssh1p, -Bos1p, -Ste14p, -Sed5p, -Sso1p, and -Snc1p. Nvi increases the resolution among the proteins of group 3. Specifically we can clearly separate Sed5p from the known membrane proteins of the ER. According to the growth of the Nvi-transformed Sec63CRUp-containing cells, Sec63p is closer to Ssh1p and Bos1p than to Sed5p and still closer to Sed5p than to Sso1p or Snc1p. We conclude that Sed5p is situated between the ER proteins, Ssh1p and Bos1p, and the proteins of the late Golgi/plasma membrane, Snc1p and Sso1p (Aalto et al., 1993; Protopopov et al., 1993). Our analysis places Ste14p between Bos1p and Sed5p.

The faint growth of the Nvi-Bos1p-containing cells in the second dilution of FIG. 6B may indicate a slightly closer proximity between Sec63p and Ssh1p than between Sec63p and Bos1p. Ssh1p is a homologue of Sec61p (FIG. 2). Ssh1p was found in a heterotrimeric complex that is very similar to the trimeric Sec6l complex. However, unlike Sec61p, Ssh1p did not copurify with the Sec62/63p complex and was not coimmunoprecipitated with antibodies to members of the Sec62/63p complex (Finke et al., 1996). Does the inability to demonstrate interaction by these techniques reflect the situation in living cells or an inherent instability of this complex that causes its disruption during purification? By comparing the growth of the Sec63CRUp cells expressing Nua-Sec61p and Nua-Ssh1p, we conclude that Sec63p is closer to Sec61p than to Ssh1p in vivo (FIG. 5 and Table 1). To confirm that the measured difference is specific and not caused by a general higher cellular activity of the Nua-Sec61p, we compared the two different Nub constructs toward a Cub landmark that is known not to interact with Sec61p or Ssh1p. We constructed a Ste14p derivative that bears the Cub-RUra3p module at its C terminus (FIG. 2, Ste14CRUp). Ste14CRUp is functional (FIG. 6A). The unimpaired growth of the Ste14CRUp-containing cells on media lacking uracil demonstrates that the Cub-RUra3p moiety most likely points into the cytosol of the cell (our unpublished observation). The nearly identical growth characteristics of the cells bearing Ste14CRUp and the Nubs of Sec62p, Sec61p, and Ssh1p document a comparable activity of the Nub fusion proteins (FIG. 6C), i.e., no growth of Ste14CRUp cells bearing the Nub, reduced but significant growth of the cells bearing the Nua, and unimpaired growth of the cells bearing the Nug constructs. We conclude that the differences in the interaction between Nua-Sec62p, -Sec61p, -Ssh1p, and Sec63CRUp are real and reflect the differences in the interaction between the unlabeled molecules. Therefore, Ssh1p is a membrane protein of the ER but does not interact with Sec63p in vivo.

FIG. 6C also shows that Ste14CRUp is closer to the Nub fusions of the ER than to the Nub fusions of any other compartment. Again, the difference between Nub-Ste14p and Nub-Sed5p is very subtle. However, we can discriminate between Sed5p and Ste14p more clearly by using the corresponding Nvis. Nvi-Ste14p is closer to Ste14CRUp than is Nvi-Sed5p (our unpublished observation). Nub-Sso1p and -Snc1p differ from the known Nub-labeled proteins of the ER and Nub-Sed5p by permitting unimpaired growth of the Ste14CRUp-containing cells (FIG. 6C and our unpublished observation).

Characterizing Proteins That Are Very Distant to Sec63p

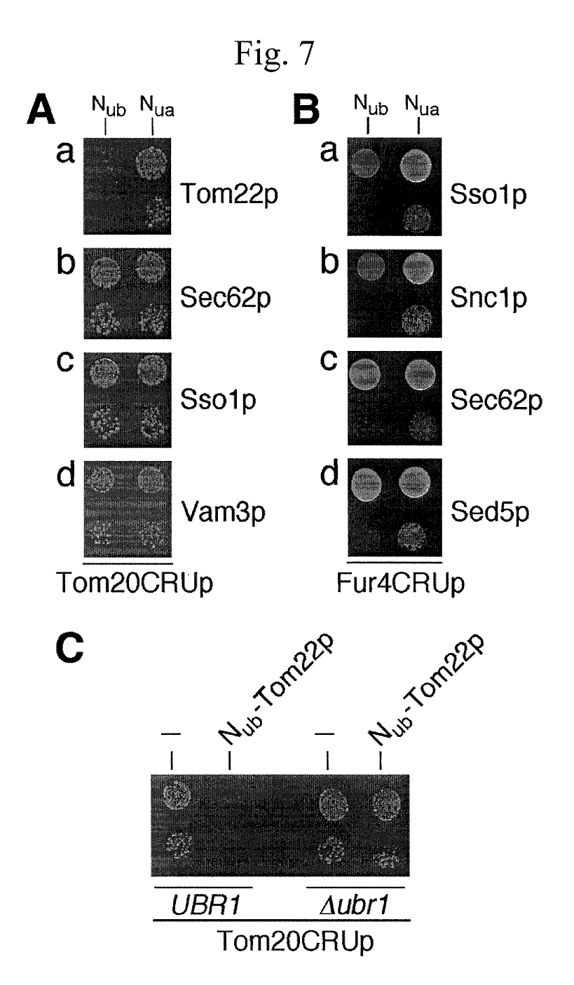

Group 4 includes the proteins whose Nub constructs impair, but do not abolish, the growth of the Sec63CRUp-containing cells. This group is very heterogeneous and thereby documents the increasing difficulty to assign a correct localization as the distance between the Cub landmark and the Nub protein gets larger (FIG. 5 and Table 1). Tom22p is localized at the outer mitochondrial membrane, while Sso1p and Snc1p, a t- and v-SNARE, are localized at the plasma membrane and the late Golgi, respectively (FIG. 2) (Aalto et al., 1993; Kiebler et al., 1993; Protopopov et al., 1993). We assumed that the assay could establish the correct localization of Nub-Tom22p, Nub-Snc1p, and Nub-Sso1p by selecting the appropriate Cub landmarks. To localize Tom22p, the Cub-RUra3p module was attached to the C terminus of Tom20p (FIG. 2, Tom20CRUp). Tom20p and Tom22p are both subunits of the translocation complex of the outer mitochondrial membrane (Schatz, 1997). Tom20p has an N-terminal membrane anchor and a C-terminal domain pointing into the cytosol of the cell (Moczko et al., 1997). Nub-Tom22p strongly impairs the growth of Tom20CRUp-containing cells on medium lacking uracil, whereas all other Nub constructs have no influence (FIG. 7A and our unpublished observation). This effect depends on a functional N-end rule pathway (FIG. 7C). We conclude that Tom22p colocalizes with Tom20p at the outer mitochondrial membrane.

FIG. 7 shows that Tom22p is close to Tom20p; and that Sso1p and Snc1p are close to Fur4p. (A) Tom20CRUp-containing S. cerevisiae cells expressing the Nub and Nua constructs of Tom22p (a), Sec62p (b), Sso1p (c), and Vam3p (d) were spotted (10³ and 10² cells) on selective media lacking uracil. Cells were grown for 3 d. (B) Fur4CRUp containing S. cerevisiae cells expressing the Nub and Nua constructs of Sso1p (a), Snc1p (b), Sec62p (c), and Sed5p (d) were spotted (10⁵ and 10³ cells) on selective media lacking uracil. Cells were grown for 3 d. (C) Tom20CRUp-containing cells bearing the UBR1 gene or a UBR1 deletion were transformed with a plasmid harboring Nub-Tom22p or the empty vector pRS314. Cells (103 and 102) were spotted on selective media lacking uracil. Plates were incubated for 3 d.

To address the localization of Sso1p and Snc1p, we constructed Fur4CRUp (FIG. 2). Fur4p belongs to the superfamily of membrane transporters, is localized in the plasma membrane, and transports uracil or 5-FOA across the membrane (Jund et al., 1988; Silve et al., 1991). The C terminus of the protein is very probably localized in the cytosol of the cell and is not important for the activity of the molecule (Jund et al., 1988). Yeast cells containing Fur4CRUp instead of the native Fur4p are still FOA sensitive, thereby demonstrating the functionality and indirectly the correct localization of the fusion protein (our unpublished observation). A subset of Nub and Nua constructs was transformed into the Fur4CRUp-expressing cells, and their growth on plates lacking uracil was scored. We observe a change in the order of proximity that was obtained for Sso1p, Snc1p, Sed5p, and Sec62p toward the Cub landmarks, Sec63p and Ste14p, of the ER. According to the growth of the Fur4CRUp-containing cells harboring the corresponding Nub constructs, Fur4p is closer to Sso1p and Snc1p than to Sed5p and Sec62p (FIG. 7B). Nub-Sec62p inhibits the growth of the Fur4CRUp-containing cells slightly more than Nub-Sed5p (FIG. 7B). Taken together, the activity of Nub-Sso1p and -Snc1p toward the landmarks, Fur4-, Sec63-, and Tom20-CRUp, is compatible with their localization at or close to the plasma membrane.

TABLE 2
Yeast strains

| Strain | Relevant genotype | Source/comment |
|---|---|---|
| JD53 | MAT his3-Δ 200 leu2-3,112 lys2-801 trp1-Δ 63 ura3-52 | Dohmen et al., 1995 |
| NJY73-I | MAT his3-Δ 200 leu2-3,112 lys2-801 trp1-Δ 63 ura3-52 | Derivative of JD53 |
| | NUB-BOS1::pRS303 | |
| NJY73-A | MAT his3-Δ 200 leu2-3,112 lys2-801 trp1-Δ 63 ura3-52 | Derivative of JD53 |
| | NUA-BOS1::pRS303 | |
| NJY73-G | MAT his3-Δ 200 leu2-3,112 lys2-801 trp1-Δ 63 ura3-52 | Derivative of JD53 |
| | NUG-BOS1::pRS303 | |
| NJY73-VI | MAT his3-Δ 200 leu2-3,112 lys2-801 trp1-Δ 63 ura3-52 | Derivative of JD53 |
| | NUVI-BOS1::pRS304 | |
| NJY61-I | MAT his3-Δ 200 leu2-3,112 lys2-801 trp1-Δ 63 ura3-52 | Derivative of JD53 |
| | NUB-SEC61::pRS304 | |
| NJY61-A | MAT his3-Δ 200 leu2-3,112 lys2-801 trp1-Δ 63 ura3-52 | Derivative of JD53 |
| | NUA-SEC61::pRS304 | |
| NJY61-G | MAT his3-Δ 200 leu2-3,112 lys2-801 trp1-Δ 63 ura3-52 | Derivative of JD53 |
| | NUG-SEC61::pRS304 | |
| NJY78-I | MAT his3-Δ 200 leu2-3,112 lys2-801 trp1-Δ 63 ura3-52 | Derivative of JD53 |
| | NUB-SSH1::pRS304 | |
| NJY78-A | MAT his3-Δ 200 leu2-3,112 lys2-801 trp1-Δ 63 ura3-52 | Derivative of JD53 |
| | NUA-SSH1::pRS304 | |
| NJY78-G | MAT his3-Δ 200 leu2-3,112 lys2-801 trp1-Δ 63 ura3-52 | Derivative of JD53 |
| | NUG-SSH1::pRS304 | |
| NJY78-VI | MAT his3-Δ 200 leu2-3,112 lys2-801 trp1-Δ 63 ura3-52 | Derivative of JD53 |
| | NUVI-SSH1::pRS304 | |
| NJY79RU | MATa/his3-Δ 200/his3-Δ 200 leu2-3,112/leu2-3,112 | Derivative of JD51 |
| | lys2-801/lys2-801 trp1-Δ 63/trp1-Δ 63 ura3-52/ura3-52 | |
| | SEC63/SEC63-CUB-RURA3::pRS305 | |
| NJY79DH | MATa/his3-Δ 200/his3-Δ 200 leu2-3,112/leu2-3,112 | Derivative of JD51 |
| | lys2-801/lys2-801 trp1-Δ 63/trp1-Δ 63 ura3-52/ura3-52 | |
| | SEC63/SEC63-CUB-DHA::pRS305 | |
| NJY80RU | MAT his3-Δ 200 leu2-3,112 lys2-801 trp1-Δ 63 | Derivative of JD53 |
| | ura3-52SEC63-CUB-RURA3:::pRS305 | |
| NJY80DH | MAT his3-Δ 200 leu2-3,112 lys2-801 trp1-Δ 63 | Derivative of JD53 |
| | ura3-52SEC63-CUB-DHA::pRS305 | |
| NJY81RU | MAT his3-Δ 200 leu2-3,112 lys2-801 trp1-Δ 63 | Ghislain et al., 1996 |
| | ura3-52SEC63-CUB-RURA3::pRS305 | |
| | UBR1::HIS3Derivative of JD55 | |
| NYJ82 | MAT his3-Δ 200 leu2-3,112 lys2-801 trp1-Δ 63 | Derivative of JD53 |
| | ura3-52FUR4-CUB-RURA3::pRS303 | |
| NJY83 | MAT ade2-1 his3-11.3-15 trp1-1 ura3-1 can1-100 | Derivative of W303 |
| | STE14::kan^{r} | |

Group 5 includes the proteins Vam3p, Tpi1p, and Guk1p. Even the Nub constructs of these proteins do not significantly impair the growth of the Sec63CRUp-bearing cells (FIG. 5 and Table 1). The Nub constructs of all three proteins were also tested against Tom20CRUp (FIG. 7A for Vam3p), Fur4CRUp, and Ste14CRUp (our unpublished observation). The proteins of this group display no significant proximity to any of the three Cub landmarks. Tpi1p and Guk1p very probably have a homogenous distribution in the cytosol and therefore are equally distant from the tested landmarks. Vam3p, as a protein of the vacuole, is in a compartment that seems to be the least accessible to all three Cub fusions (Darsow et al., 1997; Wada et al., 1997; Srivastava and Jones, 1998).

References

Aalto, M. K., Ronne, H., and Keranen, S. (1993). Yeast syntaxins Sso1p and Sso2p belong to a family of related membrane proteins that function in vesicular transport. EMBO J. 12, 4095-4104.

Ardail, D., Gasnier, F., Lerme, F., Simonot, C., Louisot, P., and Gateau-Roesch, O. (1993). Involvement of mitochondrial contact sites in the subcellular compartmentalization of phospholipid biosynthetic enzymes. J. Biol. Chem. 268, 25985-25992).

Banfield, D. K., Lewis, M. J., Rabouille, C., Warren, G., and Pelham, H. R. (1994). Localization of Sed5, a putative vesicle targeting molecule, to the cis-Go1gi network involves both its transmembrane and cytoplasmic domains. J. Cell Biol. 127, 357-37

Ballensiefen, W., Ossipov, D., and Schmitt, D. (1998). Recycling of the yeast v-SNARE Sec22p involves COPI-proteins and the ER transmembrane proteins Ufe1p and Sec20p. J. Cell Sci. 111, 1507-1520

Bartel, B., Wünning, I., and Varshavsky, A. (1990). The recognition component of the N-end rule pathway. EMBO J. 9, 3179-3189

Beckmann, R., Bubeck, D., Grassucci, R., Penczek, P., Verschoor, A., Blobel, G., and Frank, J. (1997). Alignment of conduits for the nascent polypeptide chain in the ribosome-Sec61 complex. Science 278, 2123-2126

Brodsky, J. L., and Schekman, R. (1993). A Sec63p-BiP complex from yeast is required for protein translocation in a reconstituted proteoliposome. J. Cell Biol. 123, 1355-1363

Darsow, T., Rieder, S. E., and Emr, S. D. (1997). A multispecificity syntaxin homologue, Vam3p, essential for autophagic and biosynthetic protein transport to the vacuole. J. Cell Biol. 138, 517-529

Deshaies, R. J., Sanders, S. L., Feldheim, D. A., and Schekman, R. (1991). Assembly of yeast Sec proteins involved in translocation into the endoplasmic reticulum into a membrane-bound multisubunit complex. Nature 349, 806-808

Deshaies, R. J., and Schekman, R. (1990). Structural and functional dissection of Sec62p, a membrane-bound component of the yeast endoplasmic reticulum protein import machinery. Mol. Cell. Biol. 10, 6024-6035

Dohmen, R. J., Stappen, R., McGrath, J. P., Forrova, H., Kolarov, J., Goffeau, A., and Varshavsky, A. (1995). An essential yeast gene encoding a homolog of ubiquitin-activating enzyme. J. Biol. Chem. 270, 18099-18109

Dünnwald, M., Varshavsky, A., and Johnsson, N. (1999). Detection of transient interactions between substrate and transporter during protein translocation into the endoplasmic reticulum. Mol. Biol. Cell 10, 329-344

Feldheim, D., Rothblatt, J., and Schekman, R. (1992). Topology and functional domains of Sec63p, an endoplasmic reticulum membrane protein required for secretory protein translocation. Mol. Cell. Biol. 12, 3288-3296

Finke, K., Plath, K., Panzner, S., Prehn, S., Rapoport, T. A., Hartmann, E., and Sommer, T. (1996). A second trimeric complex containing homologs of the Sec61p complex functions in protein transport across the ER membrane of S. cerevisiae. EMBO J. 15, 1482-1494

Ghislain, M., Dohmen, R. J., Levy, F., and Varshavsky, A. (1996). Cdc48p interacts with Ufd3p, a WD repeat protein required for ubiquitin-mediated proteolysis in Saccharomyces cerevisiae. EMBO J. 15, 4884-4899

Görlich, D., Prehn, S., Hartmann, E., Kalies, K. U., and Rapoport, T. A. (1992). A mammalian homolog of SEC61p and SECYp is associated with ribosomes and nascent polypeptides during translocation. Cell 71, 489-503

Güldener, U., Heck, S., Fielder, T., Beinhauer, J., and Hegemann, J. H. (1996). A new efficient gene disruption cassette for repeated use in budding yeast. Nucleic Acids Res. 24, 2519-2524

Hardwick, K. G., and Pelham, H. R. (1992). SED5 encodes a 39-kDa integral membrane protein required for vesicular transport between the ER and the Go1gi complex. J. Cell Biol. 119, 513-521

Huang, J., and Schreiber, S. L. (1997). A yeast genetic system for selecting small molecule inhibitors of protein-protein interactions in nanodroplets. Proc. Natl. Acad. Sci. USA 94, 13396-13401

Johnsson, N., and Varshavsky, A. (1994). Split ubiquitin as a sensor of protein interactions in vivo. Proc. Natl. Acad. Sci. USA 91, 10340-10344

Johnsson, N., and Varshavsky, A. (1997). Split Ubiquitin: A sensor of protein interactions in vivo. In: The Yeast Two Hybrid System, ed. P. L. Bartel, and S. Fields, Oxford, UK: Oxford University Press, 316-332.

Jund, R., Weber, E., and Chevallier, M. R. (1988). Primary structure of the uracil transport protein of Saccharomyces cerevisiae. Eur. J. Biochem. 171, 417-424

Kiebler, M., Keil, P., Schneider, H., van der Klei, I. J., Pfanner, N., and Neupert, W. (1993). The mitochondrial receptor complex: a central role of MOM22 in mediating preprotein transfer from receptors to the general insertion pore. Cell 74, 483-492

Michaelis, S., and Herskowitz, I. (1988). The a-factor pheromone of Saccharomyces cerevisiae is essential for mating. Mol. Cell. Biol. 8, 1309-1318

Moczko, M., Bömer, U., Kübrich, M., Zufall, N., Hönlinger, A., and Pfanner, N. (1997). The intermembrane space domain of mitochondrial Tom22 functions as a trans binding site for preproteins with N-terminal targeting sequences. Mol. Cell. Biol. 17, 6574-658

Ng, D. T., Brown, J. D., and Walter, P. (1996). Signal sequences specify the targeting route to the endoplasmic reticulum membrane. J. Cell Biol. 134, 269-278

Ng, D. T., and Walter, P. (1996). ER membrane protein complex required for nuclear fusion. J. Cell Biol. 132, 499-509

Paltauf, F., Kohlwein, S., and Henry, S. (1992). Regulation and compartmentalization of lipid synthesis in yeast. In: The Molecular and Cellular Biology of the Yeast Saccharomyces: Gene Expression, ed. E. J. Jones, J. Pringle, and J. Broach, Cold Spring Harbor, NY: Cold Spring Harbor Laboratory Press, 415-500.

Panzner, S., Dreier, L., Hartmann, E., Kostka, S., and Rapoport, T. A. (1995). Posttranslational protein transport in yeast reconstituted with a purified complex of Sec proteins and Kar2p. Cell 81, 561-570

Plemper, R. K., Böhmler, S., Bordallo, J., Sommer, T., and Wolf, D. H. (1997). Mutant analysis links the translocon and BiP to retrograde protein transport for ER degradation. Nature 388, 891-895

Protopopov, V., Govindan, B., Novick, P., and Gerst, J. E. (1993). Homologs of the synaptobrevin/VAMP family of synaptic vesicle proteins function on the late secretory pathway in S. cerevisiae. Cell 74, 855-861

Rapoport, T. A., Jungnickel, B., and Kutay, U. (1996). Protein transport across the eukaryotic endoplasmic reticulum and bacterial inner membranes. Annu. Rev. Biochem. 65, 271-303

Romano, J. D., Schmidt, W. K., and Michaelis, S. (1998). The Saccharomyces cerevisiae prenylcysteine carboxyl methyltransferase Ste14p is in the endoplasmic reticulum membrane. Mol. Biol. Cell 9, 2231-2247

Rothblatt, J. A., Deshaies, R. J., Sanders, S. L., Daum, G., and Schekman, R. (1989). Multiple genes are required for proper insertion of secretory proteins into the endoplasmic reticulum in yeast. J. Cell Biol. 109, 2641-2652

Rothman, J. E. (1994). Mechanisms of intracellular protein transport. Nature 372, 55-63

Sapperstein, S., Berkower, C., and Michaelis, S. (1994). Nucleotide sequence of the yeast STE14 gene, which encodes farnesylcysteine carboxyl methyltransferase, and demonstration of its essential role in a-factor export. Mol. Cell. Biol. 14, 1438-1449

Schatz, G. (1997). Just follow the acid chain. Nature 388, 121-122

Shim, J., Newman, A. P., and Ferro-Novick, S. (1991). The BOS1 gene encodes an essential 27-kDa putative membrane protein that is required for vesicular transport from the ER to the Golgi complex in yeast. J. Cell Biol. 113, 55-64

Sikorski, R. S., and Hieter, P. (1989). A system of shuttle vectors and yeast host strains designed for efficient manipulation of DNA in Saccharomyces cerevisiae. Genetics 122, 19-27

Silve, S., Volland, C., Gamier, C., Jund, R., Chevallier, M. R., and Haguenauer-Tsapis, R. (1991). Membrane insertion of uracil permease, a polytopic yeast plasma membrane protein. Mol. Cell. Biol. 11, 1114-1124

Srivastava, A., and Jones, E. W. (1998). Pth1/Vam3p is the syntaxin homolog at the vacuolar membrane of Saccharomyces cerevisiae required for the delivery of vacuolar hydrolases. Genetics 148, 85-98

Stagljar, I., Korostensky, C., Johnsson, N., and te Heesen, S. (1998). A genetic system based on split-ubiquitin for the analysis of interactions between membrane proteins in vivo. Proc. Natl. Acad. Sci. USA 95, 5187-5192

Stephenson, R. C., and Clarke, S. (1990). Identification of a C-terminal protein carboxyl methyltransferase in rat liver membranes utilizing a synthetic farnesyl cysteine-containing peptide substrate. J. Biol. Chem. 265, 16248-16254

Varshavsky, A. (1997). The N-end rule pathway of protein degradation. Genes Cells 2, 13-28

Vidal, M., Brachmann, R. K., Fattaey, A., Harlow, E., and Boeke, J. D. (1996). Reverse two-hybrid and one-hybrid systems to detect dissociation of protein-protein and DNA-protein interactions. Proc. Natl. Acad. Sci. USA 93, 10315-10320

Wada, Y., Nakamura, N., Ohsumi, Y., and Hirata, A. (1997). Vam3p, a new member of syntaxin related protein, is required for vacuolar assembly in the yeast Saccharomyces cerevisiae. J. Cell Sci. 110, 1299-1306

Walter, P., and Johnson, A. E. (1994). Signal sequence recognition and protein targeting to the endoplasmic reticulum membrane. Annu. Rev. Cell Biol. 10, 87-119

Wilkinson, B. M., Critchley, A. J., and Stirling, C. J. (1996). Determination of the transmembrane topology of yeast Sec61p, an essential component of the endoplasmic reticulum translocation complex. J. Biol. Chem. 271, 25590-25597.

Wooding, S., and Pelham, H. R. B. (1998). The dynamics of Golgi protein traffic visualized in living yeast cells. Mol. Biol. Cell 9, 2667-2680.

Zhang, F. L., and Casey, P. J. (1996). Protein prenylation: molecular mechanisms and functional consequences. Annu. Rev. Biochem. 65, 241-269

### Example 2

### Genetic Screen to Identify Transcriptional Regulator-Interacting Protein

The Saccharomyces cerevisiae GAL1 promoter is a well-studied example of transcriptional regulation by nutrients. When the cells are grown in medium containing galactose as the sole carbon source, GAL1 is activated by Gal4p, which binds specifically to the GAL1 promoter. Gal4p interacts with the holoenzyme component Srb4p, thereby recruiting the transcription apparatus to the GAL1 promoter. If the carbon source is switched to glucose, the promoter is repressed by two independently operating mechanisms. Gal80p masks the activation domain of DNA-bound Gal4p, thereby preventing the recruitment of the transcription machinery. In addition, the cytosolic repressor Mig1p enters the nucleus. Mig1p blocks transcription by recruiting the general corepressor Tup1p to its two sites in the operator region of the GAL1 promoter. Because the deletion of SRB10, a member of the RNA-PolII holoenzyme, reduces transcriptional repression by Tup1p, the repressor is thought to directly influence the transcription machinery. However, Tup1p has also been shown to bind to the histones H3 and H4, indicating that the repressor might influence transcription by altering the chromatin structure. In addition, there are other chromosomal proteins that are thought to play an architectural role in the formation of the chromatin structure: the proteins of the high mobility group (HMG). Proteins of the HMGI/Y family are necessary for the establishment of the structure of an active promoter: the enhancersome. The proteins of the HMG1 family are also involved in the negative regulation of transcription.

The classical two-hybrid screen is not suitable for the identification of interacting partners of proteins that are involved in either transcriptional activation or repression, nor is this approach suitable for the analysis of protein complexes that cannot be reconstituted in the nucleus. Therefore, we developed a generally applicable technique of screening for binding partners of proteins at any place in the cytosol of the cell. To identify additional proteins involved in the regulation of the GAL1 promoter, we carried out two split-Ub screens with Gal4p and Tup1p as baits.

Materials and Methods

Strains and Plasmids

The S. cerevisiae strains used were JD52, JD53, JD55, and NLY2. The NHP6 deletion strains were made by successive deletion of the entire NHP6A and NHP6B ORFs with the help of two knockout constructs based on NKY51. After each knockout, the URA3 gene was recombined out on 5-fluoroorotic acid (FOA) plates, and the hisG fragment remained in the place of the NHP6A and NHP6B ORFs. Consistent with previous reports, NHP6 deletion from JD52, JD53, and NLY2 caused temperature sensitivity. The NHP6 deletions were complemented by the integrative plasmids ASZ10 and YIplac128 containing PCR fragments of the NHP6A or NHP6B genes, respectively. The TUP1 deletion strains were constructed by first deleting the ADE2 gene of JD52 and JD53. An ADE2-marked PCR fragment containing 60 base pairs of the promoter and terminator sequences of TUP1 was then used to delete the entire TUP1 ORF. The REG1 deletion strains were generated by deleting the entire REG1 ORF with a HIS3-marked knockout vector. Genomic DNA was isolated from all S. cerevisiae knockout strains, and the deletions of the respective genes were verified by PCR and Southern blotting. The Escherichia coli strain used for protein purification was BL21(DE3)LysS (Stratagene). The single-copy C_{ub}-RUra3p fusion vector has been described previously. The N_{ub} fusion vectors PACNX-N_{ub}IBC and PADNX-N_{ub}IBC are single-copy and multicopy derivatives of PADNS. In these vectors, we replaced the ampicillin resistance gene with the chloramphenicol resistance gene and subcloned a PCR fragment encoding the N-terminal half of ubiquitin, a hemagglutinin (HA) tag, and a BglII site in all three reading frames under the control of the ADH1 promoter. The oligonucleotides used are: GCCAAGCTTATGCAGATTTTCGTCAAGAC, GCCAGATCTCCAGCGTAATCTGGAACA, GCCAGATCTgCCAGCGTAATCTGGAACA, and GCCAGATCTggCCAGCGTAATCTGGAACA. The single-copy C_{ub}-RGFP fusion vector was constructed by replacing the MscI/ApaI fragment containing the URA3 gene of the C_{ub}-RUra3p fusion vector with a StuI/ApaI PCR fragment containing the DNA encoding the green fluorescent protein (GFP). The oligonucleotides used here are GCCAGGCCTCATGAGTAAAGGAGAAGAACT and GCCGGGCCCTATTTGTATAGTTCATCCATGC. Following standard procedures, we generated the different fusions by cloning PCR fragments of the respective genes into the C_{ub} and N_{ub} fusion vectors. The glutathione S-transferase (GST)-Nhp6B fusion was made by cloning the NHP6B ORF into GEX-5X-1 (Amersham Pharmacia). H₆HA-Tup1p was constructed by cloning a PCR fragment containing the TUP1 ORF, six histidines, and an HA tag into pET11a (Invitrogen).

The Split-Ubiquitin Screen

The N_{ub} fusion library was made by cloning partially restricted Sau3A fragments of the ATCC library 37323 into the BglII site of PADNX-N_{ub}IBC in all three reading frames. A total of 3×10⁶ independent colonies were obtained, which suggests that the complexity of the original library (8×10⁴) was retained. A total of 5×10⁴ transformants were screened for proteins interacting with Gal4(1-147+768-881)-C_{ub}-RUra3p on FOA plates containing 100 μM CuSO₄. Four different clones were isolated, and one of them contained NHP6B. Gal80p was not isolated in this screen. In the screen using Tup1p as the C_{ub}-RUra3p bait, 10⁵ transformants were plated on medium containing FOA and 100 μM CuSO₄. Sixteen different clones were isolated, one of them as often as eight times. Two of the other clones isolated were obvious artifacts, encoding Gog5p and the related Ymd8p, small molecule transporters that confer FOA resistance when overexpressed. Yak1p, a kinase involved in cell-cycle regulation, was isolated eight times in the screen with Tup1p. It remains to be tested whether there is a biological significance for the interaction between Tup1p and Yak1p. As for the other clones isolated, their interaction will be tested for biological relevance with the help of mutants.

In Vitro Binding Assays.

The GST-fusion proteins were purified according to the protocol of the manufacturer (Amersham Pharmacia). The H₆HA-Tup1 protein was loaded onto an Ni column (Amersham Pharmacia) and eluted by increasing concentrations of imidazol. The peak fraction appeared at 250 mM imidazol. In vitro binding assays were performed as described.

β-Galactosidase Assays

Yeast strains transformed with the indicated plasmids were grown in liquid culture or on plates and assayed for β-galactosidase activity as described elsewhere. The average of at least three independent measurements is shown.

Western Blots

Western blot analysis was performed according to standard molecular biology protocols. Proteins were detected with the anti-HA antibody from Babco (Richmond, Calif.). The secondary antibody (Bio-Rad) was visualized using the ECL Western blotting detection kit (Amersham Pharmacia) following the manufacturer's protocol.

Northern Blots

Yeast RNA was isolated as described previously and incubated for 2 min at 60° C. in 1× MEN buffer (20 mM Mops/5 mM Na-acetate/1 mM EDTA, pH 7.0) containing 15% (vol/vol) formaldehyde and 50% (vol/vol) formamide. The RNA was loaded on a 0.8% agarose gel [0.8% agarose in 1× MEN buffer+5% (vol/vol) formaldehyde] and blotted overnight in 0.05 M NaOH onto a nylon membrane (Hybond N⁺, Amersham Pharmacia). The prehybridization was performed for 4 h at 42° C. in 0.25 M NaH₂PO₄, 0.25 M NaCl, 7% SDS, 1 mM EDTA, 10 mg/liter fish sperm DNA, 5% (wt/vol) PEG 6000, and 25% (vol/vol) formamide. The DNA probe was generated by PCR, purified on an agarose gel, and radioactively labeled by random hexanucleotides (Roche). The hybridization was performed overnight at 42° C., washed in 1×SSC (150 mM NaCl/15 mM Na-citrate)+0.1% SDS and analyzed by autoradiography.

Results

Split-Ub Detects the Interaction Between Gal4p and Gal80p and Between Tup1p and Ssn6p

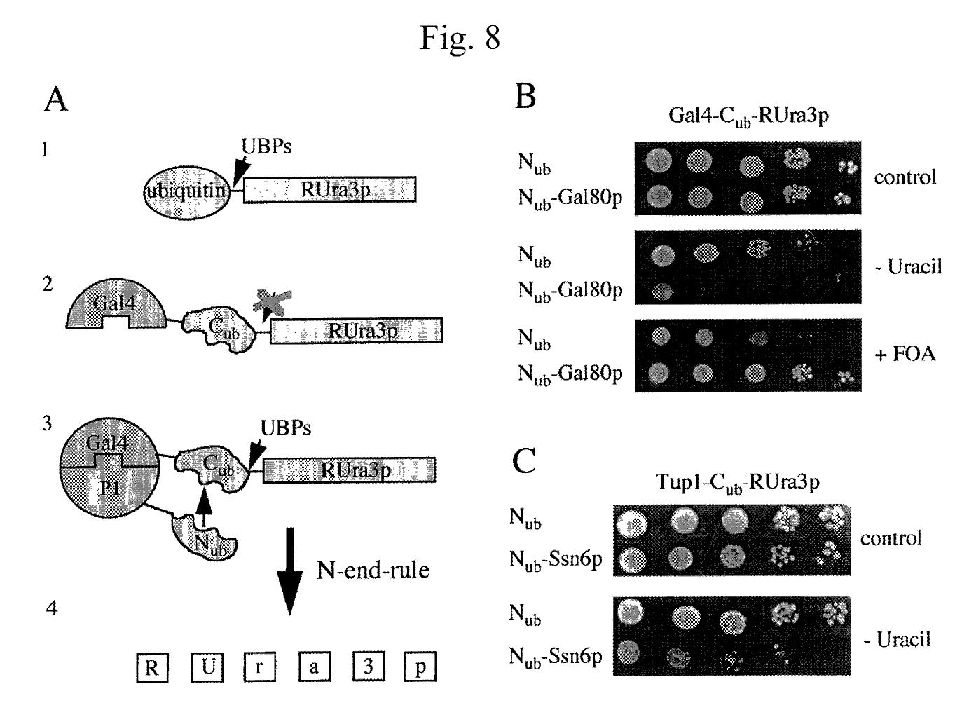

To demonstrate that split-Ub can be used to select for protein interactions that occur between transcription factors in S. cerevisiae, we first monitored the formation of the well-characterized Gal4p/Gal80p and Ssn6p/Tup1p complexes in vivo. FIG. 8A shows the conditional degradation design of the split-Ub system that was used in this study. Ubiquitin fused to a modified Ura3p with an arginine in position 1 (RUra3p) is cleaved by the UBPs (line 1). The free RUra3p is degraded rapidly because arginine is a destabilizing residue in the N-end rule pathway (line 4). A minimal Gal4p, composed of DNA-binding and activation domain only (amino acids 1-147+768-881), was fused N-terminally to C_{ub}, which was C-terminally extended by RUra3p (line 2). The Gal4-C_{ub}-RUra3 fusion protein, which is not recognized by the UBPs, is stable and enzymatically active. S. cerevisiae cells transformed with this fusion were therefore uracil prototroph and FOA sensitive (FIG. 8B). Gal80p, which is known to bind Gal4p, was fused C-terminally to N_{ub} to create N_{ub}-Gal80p. The formation of the Gal4p/Gal80p complex is expected to bring N_{ub} and C_{ub} in close proximity. The two halves of ubiquitin associate into a native-like ubiquitin, and RUra3p is cleaved off by the UBPs (FIG. 8A, line 3). The free RUra3p is degraded rapidly by the enzymes of the N-end rule pathway (FIG. 8A, line 4). Therefore, cells coexpressing Nub-Gal80p and Gal4-Cub-RUra3p were unable to grow on plates lacking uracil but were able to grow on plates containing FOA (FIG. 8B). The same experiment was repeated with isogenic cells carrying a deletion of the N-end rule pathway recognition component UBR1. These cells are unable to degrade N-end rule substrates like the cleaved RUra3p. As a consequence, the N_{ub}-Gal80p/Gal4-C_{ub}-RUra3p transformed cells retained their FOA sensitivity and were able to grow on plates lacking uracil (not shown). To test the specificity of the measured interactions, we transformed the Gal4-C_{ub}-RUra3p-containing cells with N_{ub} alone or N_{ub} coupled to the N terminus of either subunits of TFIIA (FIG. 8B and data not shown). In all three cases, no indication for an interaction with Gal4-C_{ub}-RUra3p was observed.

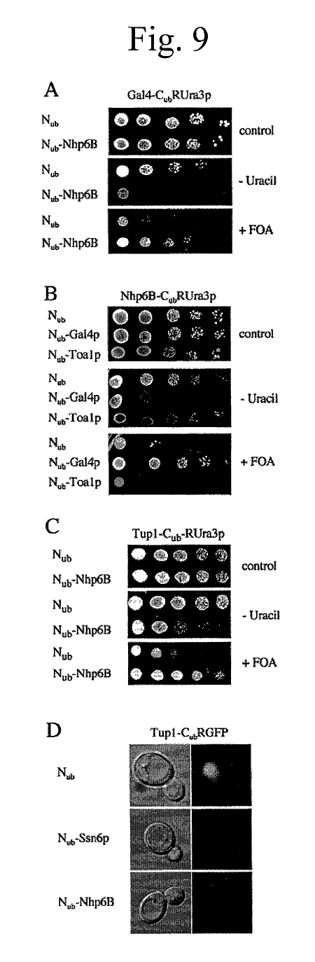

Second, a Tup1-C_{ub}-RUra3p fusion was constructed. Cells transformed with this fusion were phenotypically uracil prototroph and FOA sensitive (FIG. 8C). Ssn6p, which is known to form a complex with Tup1p, was fused to N_{ub} to create N_{ub}-ssn6p. Upon transformation of Tup1-C_{ub}-RUra3p containing cells with N_{ub}-Ssn6p, the cells became uracil auxotroph and FOA resistant. No indication for an interaction was observed between Tup1-C_{ub}-RUra3p and N_{ub} or the N_{ub} derivatives of either TFIIA subunit (FIG. 8C and data not shown), which demonstrates the specificity of the observed interaction between N_{ub}-Ssn6p and Tup1-C_{ub}-RUra3p. To verify that the interaction between N_{ub}-Ssn6p and Tup1-C_{ub}-RUra3p occurred in the nucleus, we replaced the RUra3p reporter in the Tup1p construct with a GFP module that carried the same degradation signal as RUra3p at the N terminus. Inspection of cells coexpressing N_{ub} and Tup1-C_{ub}-RGFP revealed strong nuclear green fluorescence. When the cells were coexpressing N_{ub}-Ssn⁶p instead of N_{ub}, this green fluorescence disappeared (FIG. 9D). This result strongly suggests that the observed interaction between Ssn6p and Tup1p occurs in the nucleus.

A New Split-Ub-Based Screen Identifies Nhp6 as a Binding Partner of Gal4p and Tup1p

To reveal new interaction partners of Gal4p or Tup1p, a N_{ub} library was constructed by fusing genomic S. cerevisiae Sau3A-partially digested DNA fragments in all three reading frames 3′ to the N_{ub} moiety. The N_{ub} library was transformed into a yeast strain that contained Gal4(1-147+768-881)-C_{ub}-RUra3p and into a yeast strain that contained Tup1-C_{ub}-RUra3p as a bait. After selection on FOA, the plasmids were isolated from the colony-forming cells. Only one particular ORF was discovered in both screens (FIGS. 9A and C). Because the corresponding gene promised to reveal new insights into the complex regulation of the GAL1 promoter, we focused on this particular clone. The obtained fragment encoded the 77 C-terminal residues of Nhp6B fused in frame to N_{ub}. Nhp6B is a nonhistone chromosomal protein of the HMG1 family. The isolated fragment lacks the first 22 amino acids of Nhp6B but contains the entire HMG box.

As a control, we tested the interaction between Tup1p and Nhp6B by fluorescence microscopy. Tup1-C_{ub}-RGFP was coexpressed together with N_{ub} or N_{ub}-Nhp6B. The bright nuclear fluorescence disappeared upon coexpression with N_{ub}-Nhp6B. However, the Tup1-C_{ub}-RGFP-induced fluorescence remained in the nucleus upon coexpression with N_{ub} (FIG. 9D). To find out whether Nhp6B interacts with the DNA-binding or the activation domain of Gal4p, the activation domain of Gal4(768-881) was fused behind N_{ub}, and the entire reading frame of Nhp6B was cloned in front of C_{ub}-RUra3p. Compared with the actual screen, the N_{ub}-C_{ub} arrangement was switched in this experiment. However, the interaction between the two proteins (FIG. 9B) could still be observed. This outcome not only confirmed the result of the screen, it also showed that the DNA-binding domain of Gal4p is not necessary for its interaction with Nhp6B. To test the specificity of the interaction, cells were cotransformed with Nhp6B-C_{ub}-RUra3p and N_{ub}-Toa1p, the N_{ub} fusion to the large subunit of TFIIA. Toa1p did not interact with Nhp6B in this assay (FIG. 9B), even though the interaction between the two subunits of TFIIA was readily detected (data not shown).

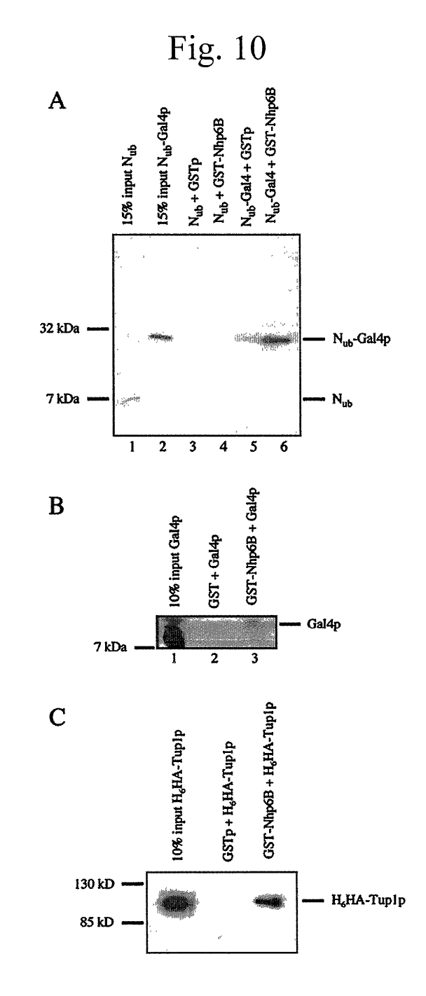

Split-Ub measures local concentration, but not necessarily a direct interaction between two proteins. To find out whether Gal4p and Nhp6 interact directly, we purified Nhp6B as a GSTp fusion from E. coli. We incubated S. cerevisiae extracts from cells expressing N_{ub} or N_{ub} fused to the activation domain of Gal4p with either GSTp or GST-Nhp6B, and the bound material was precipitated with glutathione beads. Because N_{ub} and N_{ub}-Gal4p contained the HA epitope, bound and unbound fractions were probed by anti-HA immunoblotting after SDS/PAGE. The activation domain of Gal4p was specifically precipitated with GST-Nhp6B from the extract (FIG. 10A, lane 6). Also, GST-Nhp6B precipitated the in vitro translated activation domain of Gal4p (FIG. 10B, lane 3). To test whether the measured proximity between Tup1p and Nhp6B also reflects a direct protein interaction, we fused six histidines and an HA tag to the N terminus of Tup1p. The obtained H6HA-Tup1p was purified from E. coli and incubated with purified GSTp or GST-Nhp6B attached to glutathione-Sepharose beads. H₆HA-Tup1p was only detected after SDS/PAGE by the anti HA antibody in the bound fraction of the GST-Nhp6B beads and not in the bound fraction of the GSTp beads (FIG. 10C).

Nhp6A is almost identical to Nhp6B. The presence of either protein is sufficient for proper cell growth, which indicates that Nhp6B can functionally replace Nhp6A. In contrast to Nhp6B, expression of Nhp6A from the ADH1 promoter on a multicopy vector is toxic for the cells. This explains why Nhp6A could not be isolated from the N_{ub} library. However, when we expressed the N_{ub}-Nhp6 fusions from single-copy vectors, we found that Nhp6A interacts with Gal4-C_{ub}-RUra3p and Tup1-C_{ub}-RUra3p as efficiently as Nub-Nhp6B (data not shown). The functional redundancy of the two Nhp6 proteins seems to be reflected by the redundancy of their interactions. The interactions were observed independently of Gal8Op and with and without CuSO₄ in the medium (data not shown).

The Interaction of Nhp6 with Tup1p Influences the Repression of the GAL1 Promoter

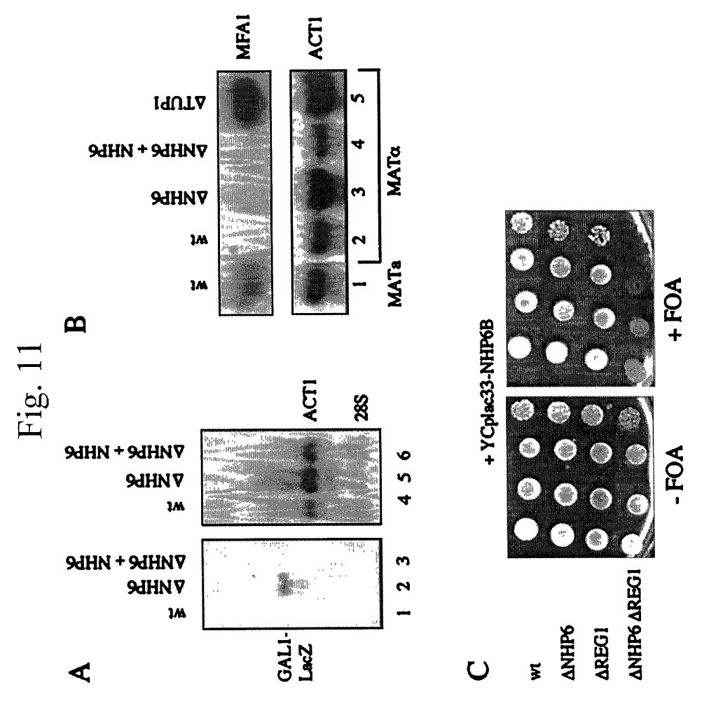

To learn more about the physiological relevance of the interaction between Nhp6 and Gal4p and between Nhp6 and Tup1p, we deleted the complete reading frames of both NHP6 genes in several strains. Because Tup1p is known to repress the GAL1 promoter in glucose-containing medium, we tested the effect of the NHP6 double deletion on the transcription of a GAL1-LacZ reporter gene. When the cells were grown in glucose, we measured 0.51 Error! Unknown switch argument.-galactosidase units for the wild-type strain and 5.3 units for the NHP6 deletion strain. The isogenic strain deleted for TUP1 yielded 12.7 units. We performed a Northern blot with a LacZ probe and demonstrated that the loss of glucose repression took place at the level of transcription (FIG. 11A). The increased amount of the GAL1-LacZ mRNA in the NHP6 deletion strain (compare lanes 1 and 2) were reduced to wild-type levels upon reintegration of NHP6 (lane 3). We also tested the expression of the glucose-repressed SUC2 promoter in our deletion strains. As has been shown for the GAL1-LacZ transcription, the integrated SUC2-LacZ reporter showed reduction of glucose repression in the NHP6 deletion strain as well as in the strain lacking TUP1 (data not shown). Besides regulating glucose-responsive genes, Tup1p is also involved in the repression of MFA1 in MATα cells. Interestingly, Nhp6 does not seem to be involved in the Tup1p-mediated α2p repression (FIG. 11B). Although the deletion of TUP1 resulted in derepression of MFA1 in MATα cells, the deletion of NHP6 had no effect (compare lanes 2, 3, and 5). A similar pattern was observed for the expression of the α2-regulated STE2. A STE2-LacZ fusion was up-regulated in the TUP1 deletion strain but was still repressed in the NHP6 deletion strain (data not shown). Cells that are deficient for Tup1p display a flocculent phenotype. This phenotype was not observed for cells lacking Nhp6. These observations indicate that Nhp6 acts together with Tup1p specifically on the glucose-regulated promoters GAL1 and SUC2. However, unlike Tup1p, Nhp6 is not involved in the repression of the mating type-specific promoters MFA1 and STE2.

Synthetic Lethality Between NHP6 and REG1

Not to rely exclusively on experiments with artificial promoter fusion constructs, we tried to delete REG1. REG1 causes the degradation of glucose-repressed mRNAs by XRN1 in glucose. A REG1 deletion should therefore allow to measure the effect of the NHP6 deletion on the transcription of the natural GAL1 and SUC2 genes. However, several independent strains chromosomally deficient for NHP6A, NHP6B, and REG1, which carried NHP6B on a URA3-marked plasmid, were unable to lose this plasmid and therefore unable to grow on FOA (FIG. 11C). This experiment shows that simultaneous deletion of REG1 and NHP6 is lethal to the cells and provides an independent link between NHP6 and glucose repression.

The Interaction of Nhp6 with Gal4p Influences the Activation of the GAL1 Promoter

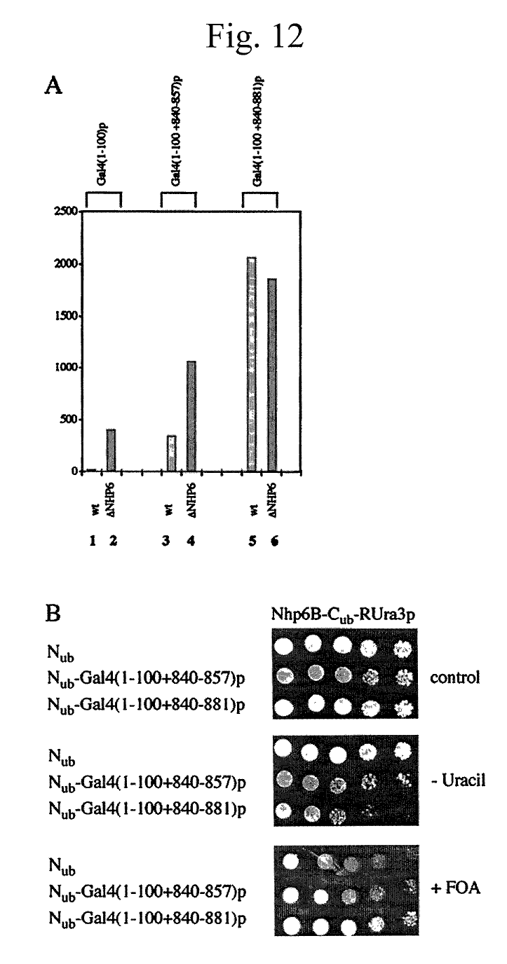

In contrast to published findings, we could not measure a decrease in the activation potential of Gal4p in cells lacking NHP6. We reasoned that Gal4p, as an activator of transcription, might be simply too strong to yield a significant effect of Nhp6 on the transcription of the reporter genes. We therefore compared the ability of Gal4p derivatives that lacked parts of the activation domain to stimulate transcription in strains containing or lacking NHP6. The Gal4p derivatives were expressed as N_{ub} fusions from the constitutive ADH1 promoter. This enabled us to test the same molecule for both transcriptional activation and interaction in vivo. NHP6 was deleted from the S. cerevisiae strain NLY2, which is deficient for GAL4 and GAL80. A GAL1 -LacZ fusion was integrated into the GAL1 locus of the NLY2 wild-type and NHP6 deletion strains. The strains were transformed with the plasmids expressing the Gal4p derivatives, and cells were grown in glucose. FIG. 12A shows transcriptional activation of a GAL1 -LacZ fusion by three different N_{ub}-Gal4p derivatives. Increasing the size of the deletion within the activation domain corresponded to a decrease in the transcription of the LacZ reporter, and this effect was seen independently of NHP6. However, there was a clear difference in the extent of activation between the NHP6-containing and NHP6-lacking strains. The N_{ub}-Gal4p derivative that has no or only a severely truncated activation domain stimulated transcription from the GAL1 promoter significantly better in a strain that lacks NHP6 (compare lanes 3 and 4). This difference was not observed for the N_{ub}-Gal4p fusion that harbored the complete activation domain (compare lanes 5 and 6). The ability to activate transcription in the strain carrying NHP6 correlated with the ability of the two N_{ub}-Gal4p derivatives to interact with Nhp6B-C_{ub}-RUra3p. The Gal4p derivative with the truncated activation domain interacted less efficiently with Nhp6B than the protein with the intact activation domain (FIG. 12B). We suggest that one additional function of the activation domain of Gal4p is to contact and to remove Nhp6 or remodel its position on the chromatin structure.

Discussions

Yeast two-hybrid screens have been successfully used to isolate binding partners of proteins fused to a DNA-binding domain. However, proteins that activate or repress transcription in S. cerevisiae cannot be used as baits because the signal of the two-hybrid screen itself is based on the transcriptional readout of a reporter protein. The split-ubiquitin system makes use of the facilitated reassociation of the two ubiquitin halves and the subsequent cleavage by the UBPs. As a consequence, transcriptional regulators do not interfere with the readout and can be used as baits in a screen. This rational was confirmed in the work presented here. In a two-step approach, we first showed that split-Ub can monitor the interaction between transcription factors by following the formation of the Gal4p/Gal80p and of the Ssn6p/Tup1p complexes in vivo. Cells expressing a Gal4-C_{ub}-RUra3p fusion or a Tup1-C_{ub}-RUra3p fusion display a ura-phenotype only if an N_{ub}-Gal80p or an N_{ub}-Ssn6p fusion is coexpressed. Second, we have shown that split-Ub can be used to screen N_{ub} fusion libraries for proteins that interact with a given C_{ub}-RUra3p bait. Using the two known regulators of the GAL1 promoter, Gal4p and Tup1p, as C_{ub}-RUra3p baits, we have isolated the HMG box of the chromosomal protein Nhp6B in both screens. Interaction was also observed for full-length Nhp6B, which demonstrates that at least in this case, structural constraints are not limiting the split-Ub system. Because split-Ub measures the local concentration of the N_{ub}- and C_{ub}-coupled proteins, it was important to biochemically determine the nature of this proximity. Using GSTp pull-down assays, a direct interaction between Nhp6 and Tup1p and between Nhp6 and Gal4p was established. Furthermore, we have shown that the observed protein interactions are biologically relevant for the regulation of the GAL1 promoter.

The approach introduced here will also allow to screen for binding partners of proteins that are not localized in the nucleus. There are now different C_{ub}-RUra3 fusion proteins available that are cytosolic or directed to the membrane of the endoplasmic reticulum, the outer mitochondrial membrane, the membrane of the peroxisome, or the plasma membrane (J. H. Eckert and N. J., unpublished data). The scarcity of methods to analyze membrane proteins makes this system particularly attractive.

To be able to confirm the localization of the Cub-modified proteins, we have created an N-end rule-sensitive GFP reporter for the split-Ub system. Using this assay, Tup1-C_{ub}-RGFP localized in the nucleus of the cells. The fluorescence disappears upon introduction of the N_{ub} versions of the two Tup1p binding partners Ssn6p and Nhp6B. This feature of the new reporter will give us the opportunity to better follow the dynamics of protein interactions in living cells or monitor signals that induce or terminate a specific protein interaction.

Equivalents

Those skilled in the art will recognize, or be able to ascertain using no more than routine experimentation, numerous equivalents to the specific procedures described herein. Such equivalents are considered to be within the scope of this invention and are covered by the following claims.

We claim:

1. A pair of fusion proteins consisting of a first fusion protein comprising segments P1, Cub-X, and RM, in an order wherein Cub-X is closer to the N-terminus of the first fusion protein than RM, and a second fusion protein comprising segments Nux and P2, wherein:
P1 or P2 or both is a membrane-associated protein, and P2 may be the same or different from P1;
Nux is the amino-terminal subdomain of a wild-type ubiquitin or a reduced-associating mutant ubiquitin amino-terminal subdomain;
Cub is the carboxy-terminal subdomain of a wild-type ubiquitin;
X is an amino acid other than methionine;
RM is an reporter moiety, and,
wherein the binding that occurs between P1 and P2 results in reassociation of Nux and Cub, thereby permitting ubiquitin-specific protease cleavage between Cub and X.

2. A pair of fusion proteins consisting of a first fusion protein comprising segments P1, Cub-X, and RM, in an order wherein Cub-X is closer to the N-terminus of the first fusion protein than RM, and a second fusion protein comprising segments Nux and P2, wherein:
P1 or P2 or both is a transcription factor;
Nux is the amino-terminal subdomain of a wild-type ubiquitin or a reduced-associating mutant ubiquitin amino-terminal subdomain;
Cub is the carboxy-terminal subdomain of a wild-type ubiquitin;
X is an amino acid other than methionine;
RM is an reporter moiety, and,
wherein the binding that occurs between P1 and P2 results in reassociation of Nux and Cub, thereby permitting ubiquitin-specific protease cleavage between Cub and X.

3. A pair of fusion proteins consisting of a first fusion protein comprising segments P1, Cub-X, and RM, in an order wherein Cub-X is closer to the N-terminus of the first fusion protein than RM, and a second fusion protein comprising segments Nux and P2, wherein:
P1 or P2 or both is a membrane-associated protein, and P2 may be the same or different from P1;
Nux is the amino-terminal subdomain of a wild-type ubiquitin or a reduced-associating mutant ubiquitin amino-terminal subdomain;
Cub is the carboxy-terminal subdomain of a wild-type ubiquitin;
X is an amino acid;
RM is an enzymatically active reporter moiety, and,
wherein the binding that occurs between P1 and P2 results in reassociation of Nux and Cub, thereby permitting ubiquitin-specific protease cleavage between Cub and X.

4. A pair of fusion proteins consisting of a first fusion protein comprising segments P1, Cub-X, and RM, in an order wherein Cub-X is closer to the N-terminus of the first fusion protein than RM, and a second fusion protein comprising segments Nux and P2, wherein:
P1 or P2 or both is transcription factor;
Nux is the amino-terminal subdomain of a wild-type ubiquitin or a reduced-associating mutant ubiquitin amino-terminal subdomain;
Cub is the carboxy-terminal subdomain of a wild-type ubiquitin;
X is an amino acid;
RM is an enzymatically active reporter moiety, and,
wherein the binding that occurs between P1 and P2 results in reassociation of Nux and Cub, thereby permitting ubiquitin-specific protease cleavage between Cub and X.

5. The pair of fusion proteins of claim 1, 2, 3, or 4, wherein X is Arginine.

6. The pair of fusion proteins of claim 1, 2, 3, or 4, wherein X is selected from the group consisting of Lysine, Histidine, Phenylalanine, Tryptophan, Tyrosine, Leucine, Aspartate, Glutamate, Cysteine, Asparagine, Glutamine and Isoleucine.

7. The pair of fusion proteins of claim 3 or 4, wherein X is Methionine, Glycine or Valine.

8. The pair of fusion proteins of claim 1, 2, 3, or 4, wherein the reporter moiety is a selectable marker.

9. The pair of fusion proteins of claim 8, wherein the selectable marker is selected from the group consisting of: URA3, HIS3, LYS2, HygTk, Tkneo, TkBSD, PACTk, HygCoda, Codaneo, CodaBSD, PACCoda, Tk, codA, GPT2, and HPRT.

10. The pair of fusion proteins of claim 8, wherein the selectable marker is selected from the group consisting of: TRP1, CYH2, CAN1.

11. The pair of fusion proteins of claim 1 or 2, wherein the reporter moiety is selected from the group consisting of: a transcription factor and a fluorescent marker.

12. The pair of fusion proteins of claim 1, 2, 3, or 4, wherein Nux contains at least one point mutation at amino acid 3 or amino acid 13 of a ubiquitin.

13. One or more nucleic acids that encodes or that together encode a first fusion protein comprising segments P1, Cub-X, and RM, in an order wherein Cub-X is closer to the N-terminus of the first fusion protein than RM, and a second fusion protein comprising segments Nux and P2, wherein:
P1 or P2 or both is a membrane-associated protein, and P2 may be the same or different from P1;
Nux is the amino-terminal subdomain of a wild-type ubiquitin or a reduced-associating mutant ubiquitin amino-terminal subdomain;
Cub is the carboxy-terminal subdomain of a wild-type ubiquitin;
X is an amino acid other than methionine;
RM is a reporter moiety, and,
wherein the binding that occurs between P1 and P2 results in reassociation of Nux and Cub, thereby permitting ubiquitin-specific protease cleavage between Cub and X.

14. One or more nucleic acids that encodes or that together encode a first fusion protein comprising segments P1, Cub-X, and RM, in an order wherein Cub-X is closer to the N-terminus of the first fusion protein than RM, and a second fusion protein comprising segments Nux and P2, wherein:
P1 or P2 or both is a membrane-associated protein, and P2 may be the same or different from P1;
Nux is the amino-terminal subdomain of a wild-type ubiquitin or a reduced-associating mutant ubiquitin amino-terminal subdomain;
Cub is the carboxy-terminal subdomain of a wild-type ubiquitin;
X is an amino acid;
RM is an enzymatically active reporter moiety, and,
wherein the binding that occurs between P1 and P2 results in reassociation of Nux and Cub, thereby permitting ubiquitin-specific protease cleavage between Cub and X.

15. One or more nucleic acids that encodes or that together encode a first fusion protein comprising segments P1, Cub-X, and RM, in an order wherein Cub-X is closer to the N-terminus of the first fusion protein than RM, and a second fusion protein comprising segments Nux and P2, wherein:
P1 or P2 or both is a transcription factor;
Nux is the amino-terminal subdomain of a wild-type ubiquitin or a reduced-associating mutant ubiquitin amino-terminal subdomain;
Cub is the carboxy-terminal subdomain of a wild-type ubiquitin;
X is an amino acid other than methionine;
RM is a reporter moiety, and,
wherein the binding that occurs between P1 and P2 results in reassociation of Nux and Cub, thereby permitting ubiquitin-specific protease cleavage between Cub and X.

16. One or more nucleic acids that encodes or that together encode a first fusion protein comprising segments P1, Cub-X, and RM, in an order wherein Cub-X is closer to the N-terminus of the first fusion protein than RM, and a second fusion protein comprising segments Nux and P2, wherein:
P1 or P2 or both is a transcription factor;
Nux is the amino-terminal subdomain of a wild-type ubiquitin or a reduced-associating mutant ubiquitin amino-terminal subdomain;
Cub is the carboxy-terminal subdomain of a wild-type ubiquitin;
X is an amino acid;
RM is an enzymatically active reporter moiety, and,
wherein the binding that occurs between P1 and P2 results in reassociation of Nux and Cub, thereby permitting ubiquitin-specific protease cleavage between Cub and X.

17. A method of determining whether two proteins, at least one of which is a membrane-associated protein, bind to each other comprising the steps of:
translationally providing a first fusion protein comprising segments P1, Cub-X, and RM, in an order wherein Cub-X is closer to the N-terminus of the first fusion protein than RM, and a second fusion protein comprising segments Nux and P2, wherein P1 and P2 are proteins, at least one of which is membrane-associated, which proteins may be the same or different, Nux is the amino-terminal subdomain of a wild-type ubiquitin or a reduced-associating mutant ubiquitin amino-terminal subdomain, Cub is the carboxy-terminal subdomain of a wild-type ubiquitin, X is an amino acid other than methionine and RM is an active reporter moiety; and
detecting the degree of cleavage by a ubiquitin-specific protease of the first fusion protein between Cub and X by detecting the degree of the activity of RM, wherein an increase of cleavage is indicative of P1/P2 binding.

18. A method of determining whether two proteins, at least one of which is a transcription factor, bind to each other comprising the steps of:
translationally providing a first fusion protein comprising segments P1, Cub-X, and RM, in an order wherein Cub-X is closer to the N-terminus of the first fusion protein than RM, and a second fusion protein comprising segments Nux and P2, wherein P1 and P2 are proteins, at least one of which is a transcription factor, Nux is the amino-terminal subdomain of a wild-type ubiquitin or a reduced-associating mutant ubiquitin amino-terminal subdomain, Cub is the carboxy-terminal subdomain of a wild-type ubiquitin, X is an amino acid other than methionine and RM is an active reporter moiety; and
detecting the degree of cleavage by a ubiquitin-specific protease of the first fusion protein between Cub and X by detecting the degree of the activity of RM, wherein an increase of cleavage is indicative of P1 /P2 binding.

19. A method of determining whether two proteins bind to each other, at least one of which is a membrane-associated protein, comprising the steps of:
translationally providing a first fusion protein comprising segments P1, Cub-X, and RM, in an order wherein Cub-X is closer to the N-terminus of the first fusion protein than RM, and a second fusion protein comprising segments Nux and P2, wherein P1 and P2 are proteins, at least one of which is membrane-associated, which proteins may be the same or different, Nux is the amino-terminal subdomain of a wild-type ubiquitin or a reduced-associating mutant ubiquitin amino-terminal subdomain, Cub is the carboxy-terminal subdomain of a wild-type ubiquitin, X is an amino acid and RM is an enzymatically active reporter moiety; and
detecting the degree of cleavage by a ubiquitin-specific protease of the first fusion protein between Cub and X by detecting the degree of the enzymatic activity of RM, wherein an increase of cleavage is indicative of P1/P2 binding.

20. A method of determining whether two proteins bind to each other, at least one of which is a transcription factor, comprising the steps of:
translationally providing a first fusion protein comprising segments P1, Cub-X, and RM, in an order wherein Cub-X is closer to the N-terminus of the first fusion protein than RM, and a second fusion protein comprising segments Nux and P2, wherein P1 and P2 are proteins, at least one of which is a transcription factor, Nux is the amino-terminal subdomain of a wild-type ubiquitin or a reduced-associating mutant ubiquitin amino-terminal subdomain, Cub is the carboxy-terminal subdomain of a wild-type ubiquitin, X is an amino acid and RM is an enzymatically active reporter moiety; and,
detecting the degree of cleavage by a ubiquitin-specific protease of the first fusion protein between Cub and X by detecting the degree of the enzymatic activity of RM, wherein an increase of cleavage is indicative of P1 /P2 binding.

21. The method of claim 17, 18, 19, or 20, wherein X is selected from the group consisting of Arginine, Lysine, Histidine, Phenylalanine, Tryptophan, Tyrosine, Leucine, Aspartate, Glutamate, Cysteine, Asparagine, Glutamine and Isoleucine.

22. The method of claim 19 or 20, wherein X is Methionine, Glycine or Valine.

23. The method of claim 17 or 18, wherein the reporter moiety is selected from the group consisting of: a transcription factor and a fluorescent marker.

24. The method of claim 17, 18, 19, or 20, wherein the translationally providing step is performed by a cell that expresses the ubiquitin-specific protease.

25. The method of claim 17, 18, 19, or 20, wherein the translationally providing step and the step wherein cleavage between Cub and X may occur is performed by a cell that expresses the ubiquitin-specific protease.

26. The method of claim 25, wherein the cell is a eukaryotic cell.

27. The method of claim 25, wherein the cell is a mammalian cell.

28. The method of claim 25, wherein the cell is a fungal cell.

29. The method of claim 25, wherein the cell is a plant cell.

30. The method of claim 25, wherein the cell is an insect cell.

31. The method of claim 25, wherein the cell is selected from the group consisting of a human cell, a mouse cell, a rat cell, a hamster cell, a zebrafish cell, a Drosophila cell, a nematode cell, an S. pombe cell and an S. cerevisiae cell.

32. The method of claim 25, wherein the cell is selected from the group consisting of: an A. thaliana cell and an N. tabacum cell.

33. The method of claim 25, wherein the reporter moiety is a negative selectable marker, and the degree of activity of the reporter moiety is determined by incubating the cell under conditions that select against the negative selectable marker so that continued viability of the cell under negative selection conditions indicates that P1 binds P2.

34. The method of claim 33, wherein the negative selectable marker is selected from the group consisting of: URA3, Tk, codA, HygTk, Tkneo, TkBSD, PACTk, HygCoda, Codaneo, CodaBSD, PACCoda, GPT2, and HPRT.

35. The method of claim 33, wherein the negative selectable marker is selected from the group consisting of: TRP1, CYH2, CAN1.

36. The method of claim 25, wherein the reporter moiety is a positive selectable marker, and the presence or absence of the reporter moiety is determined by comparing the viability of the cell under conditions that select for the positive selectable marker to the viability of the cell under nonselective conditions, so that decreased viability of the cell grown under the positive selection conditions as compared to the viability of the cell grown under the nonselective conditions indicates that P1 binds P2.

37. The method of claim 36, wherein the positive selectable marker is selected from the group consisting of: URA3, Tk, Hygromycinresistance (Hyg^{R}), neomycin resistance (neo^{R}), puromycin resistance (PAC^{R}) or Blasticidin S resistance (BlaS^{R}), codA, HygTk, Tkneo, TkBSD, PACTk, HygCoda, Codaneo, CodaBSD, PACCoda, and GPT2.

38. The method of claim 36, wherein the positive selectable marker is selected from the group consisting of: HIS3, LYS2, LEU2, TRP2, ADE2.

39. The method of claim 17, 18, 19, or 20, wherein Nux contains at least one point mutation at amino acid 3 or amino acid 13 of a ubiquitin.

40. A method of determining whether a test compound agonizes or antagonizes the binding of two proteins to each other comprising the steps of:
translationally providing a first fusion protein comprising segments P1, Cub-X, and RM, in an order wherein Cub-X is closer to the N-terminus of the first fusion protein than RM, and a second fusion protein comprising segments Nux and P2, wherein P1 and P2 are proteins, which proteins may be the same or different, Nux is the amino-terminal subdomain of a wild-type ubiquitin or a reduced-associating mutant ubiquitin amino-terminal subdomain, Cub is the carboxy-terminal subdomain of a wild-type ubiquitin, X is an amino acid other than methionine and RM is an active reporter moiety; and,
comparing the amount of cleavage by a ubiquitin-specific protease between Cub and X by detecting the degree of the activity of RM in the presence of the compound with the amount of such cleavage that is expected in the absence of the test compound or in the presence of a standard compound, wherein increased cleavage indicates the test compound is an agonist and decreased cleavage indicates the test compound is an antagonist of P1/P2 binding.

41. A method of determining whether a test compound agonizes or antagonizes the binding of two proteins to each other comprising the steps of:
translationally providing a first fusion protein comprising segments P1, Cub-X, and RM, in an order wherein Cub-X is closer to the N-terminus of the first fusion protein than RM, and a second fusion protein comprising segments Nux and P2, wherein P1 and P2 are proteins, which proteins may be the same or different, Nux is the amino-terminal subdomain of a wild-type ubiquitin or a reduced-associating mutant ubiquitin amino-terminal subdomain, Cub is the carboxy-terminal subdomain of a wild-type ubiquitin, X is an amino acid and RM is an enzymatically active reporter moiety; and,
comparing the amount of cleavage by a ubiquitin-specific protease between Cub and X by detecting the degree of the enzymatic activity of RM in the presence of the compound with the amount of such cleavage that is expected in the absence of the test compound or in the presence of a standard compound, wherein increased cleavage indicates the test compound is an agonist and decreased cleavage indicates the test compound is an antagonist of P1/P2 binding.

42. A method for selecting an agonist or antagonist of P1/P2 binding from a library of test compounds, a multiplicity of said library compounds having no known agonist or antagonist activity for P1/P2 binding, comprising:
1) determining the agonist or antagonist activity of each test compound of the library according to the method of claim 40 or 41; and,
2) selecting from the multiplicity at least one test compound that shows agonistic or antagonistic activity.

43. The method of claim 40 or 41, further comprising: selecting a candidate compound from a library of candidates which comprise 10 to 500 compounds, wherein multiple members of said library are not known to bind P1 or P2.

44. The method of claim 40 or 41, further comprising: selecting a candidate compound from a library of candidates which comprise 500 to 10,000 compounds, wherein multiple members of said library are not known to bind P1 or P2.

45. The method of claim 40 or 41, further comprising: selecting a candidate compound from a library of candidates which comprise greater than 10,000 compounds, wherein multiple members of said library are not known to bind P1 or P2.

46. The method of claim 42, wherein said library of candidate compounds is selected from the group: synthetic chemical library and natural chemical library.

47. The method of claim 40 or 41, wherein the candidate compound is a polypeptide.

48. The method of claim 47, wherein said polypeptide is supplied by a polypeptide library.

49. The method of claim 40 or 41, wherein the candidate compound is a small molecule compound.

50. The method of claim 40 or 41, wherein X is selected from the group consisting of:
Arginine, Lysine, Histidine, Phenylalanine, Tryptophan, Tyrosine, Leucine, Aspartate, Glutamate, Cysteine, Asparagine, Glutamine and Isoleucine.

51. The method of claim 41, wherein X is Methionine, Glycine or Valine.

52. The method of claim 40 or 41, wherein the reporter moiety is a selectable marker.

53. The method of claim 52, wherein the selectable marker is selected from the group consisting of: URA3, HIS3, LYS2, HygTk, Tkneo, TkBSD, PACTk, HygCoda, Codaneo, CodaBSD, PACCoda, Tk, coda, GPT2, and HPRT.

54. The method of claim 52, wherein the selectable marker is selected from the group consisting of: TRP1, CYH2, CAN1.

55. The method of claim 40 or 41, wherein the reporter moiety is selected from the group consisting of: a transcription factor and a fluorescent marker.

56. The method of claims 40 or 41, wherein the translationally providing step is performed by a cell that expresses the ubiquitin-specific protease.

57. The method of claim 56, wherein the translationally providing step and the step wherein cleavage between Cub and X may occur is performed by a cell that expresses the ubiquitin-specific protease.

58. The method of claim 57, wherein the cell is a eukaryotic cell.

59. The method of claim 57, wherein the cell is a mammalian cell.

60. The method of claim 57, wherein the cell is a fungal cell.

61. The method of claim 57, wherein the cell is a plant cell.

62. The method of claim 57, wherein the cell is an insect cell.

63. The method of claim 57, wherein the cell is selected from the group consisting of: a human cell, a mouse cell, a rat cell, a hamster cell, a zebrafish cell, a Drosophila cell, a nematode cell, an S. pombe cell and an S. cerevisiae cell.

64. The method of claim 57, wherein the cell is selected from the group consisting of: an A. thaliana cell and an N. tabacum cell.

65. The method of claim 40 or 41, wherein Nux contains at least one point mutation at amino acid 3 or amino acid 13 of a ubiquitin.

66. A method of characterizing the sequence of a protein that binds a target protein comprising the steps of:
expressing a first and a second nucleic acid in a ubiquitin-specific protease expressing cell, which first nucleic acid encodes a target fusion protein comprising segments P1, Cub-X, and RM, in an order wherein Cub-X is closer to the N-terminus of the target fusion protein than RM, wherein P1 is the target protein, Cub is the carboxy-terminal subdomain of a wild-type ubiquitin, X is an amino acid selected from the group consisting of arg, lys, phe, leu, trp, his, asp, asn, tyr, ile, glu, cys and gln, and RM is an enzymatically active reporter moiety,
which second nucleic acid encodes a candidate fusion protein comprising segments P2 and Nux, wherein the second nucleic acid is a member of a library containing multiple different nucleic acids differing in the P2 segments they encode, P2 is a candidate segment and Nux is the amino-terminal subdomain of a wild-type ubiquitin or a reduced-associating mutant ubiquitin amino-terminal subdomain;
recovering a clone of the cell expressing the first and second nucleic acid under conditions wherein a cell is selectable only in the absence of the enzymatic activity of RM; and,
characterizing the second nucleic acid encoding P2.

67. The method of claim 66, wherein the enzymatically active reporter moiety is a negative selectable marker selected from the group consisting of: URA3, Tk, codA, HygTk, Tkneo, TkBSD, PACTk, HygCoda, Codaneo, CodaBSD, PACCoda, GPT2, and HPRT.

68. The method of claim 66, wherein the enzymatically active reporter moiety is a negative selectable marker selected from the group consisting of: TRP1, CAN1 and CYH2.

69. A method of characterizing the sequence of a protein that binds a target protein comprising the steps of:
expressing a first and a second nucleic acid in a ubiquitin-specific protease expressing cell, which first nucleic acid encodes a target fusion protein comprising segments P1, Cub-X, and RM, in an order wherein Cub-X is closer to the N-terminus of the target fusion protein than RM, wherein P1 is the target protein, Cub is the carboxy-terminal subdomain of a wild-type ubiquitin, X is an amino acid selected from the group consisting of arg, lys, phe, leu, trp, his, asp, asn, tyr, ile, glu, cys and gln, and RM is an active reporter moiety, which second nucleic acid encodes a candidate fusion protein comprising segments P2 and Nux, wherein the second nucleic acid is a member of a library containing multiple different nucleic acids differing in the P2 segments they encode, P2 is a candidate segment and Nux is the amino-terminal subdomain of a wild-type ubiquitin or a reduced-associating mutant ubiquitin amino-terminal subdomain;
recovering a clone of the cell expressing the first and second nucleic acid under conditions wherein a cell is selectable only in the absence of an activity of RM; and,
characterizing the second nucleic acid encoding P2.

70. The method of claim 69, wherein the active reporter moiety is selected from the group consisting of: a transcription factor and a fluorescent marker.

71. The method of claim 66 or 69, wherein the cell is a eukaryotic cell.

72. The method of claim 66 or 69, wherein the cell is a mammalian cell.

73. The method of claim 66 or 69, wherein the cell is a fungal cell.

74. The method of claim 66 or 69, wherein the cell is a plant cell.

75. The method of claim 66 or 69, wherein the cell is an insect cell.

76. The method of claim 66 or 69, wherein the cell is selected from the group consisting of: a human cell, a mouse cell, a rat cell, a hamster cell, a zebrafish cell, a Drosophila cell, a nematode cell, an S. pombe cell and an S. cerevisiae cell.

77. The method of claim 66 or 69, wherein the cell is selected from the group consisting of: an A. thaliana cell and an N. tabacum cell.

78. The method of claim 66 or 69, wherein the library of nucleic acids comprises 10 to 500 members, wherein fusions proteins encoded by multiple members of said library are not known to bind P1.

79. The method of claim 66 or 69, wherein the library of nucleic acids comprises 500 to 10,000 members, wherein fusions proteins encoded by multiple members of said library are not known to bind P1.

80. The method of claim 66 or 69, wherein the library of nucleic acids comprises greater than 10,000 members, wherein fusions proteins encoded by multiple members of said library are not known to bind P1.

81. The method of claim 66 or 69, wherein Nux contains at least one point mutation at amino acid 3 or amino acid 13 of a ubiquitin.

82. A kit for characterizing the sequence of a polypeptide that binds a target protein, which comprises:
a first nucleic acid encoding a target fusion protein comprising a cloning site suitable for the insertion of a nucleic acid encoding a target protein sequence, segments Cub-X, and RM, in an order wherein Cub-X is closer to the N-terminus of the target fusion protein than RM, wherein Cub is the carboxy-terminal subdomain of a wild-type ubiquitin, X is an amino acid selected from the group consisting of arg, lys, phe, leu, trp, his, asp, asn, tyr, ile, glu, cys and gln, and RM is an active reporter moiety, which activity allows for selection, whereby a fusion protein comprising the target protein sequence, Cub-X and RM can be expressed;
a second nucleic acid comprising an Nux segment encoding the amino-terminal subdomain of a wild-type ubiquitin or a reduced-associating mutant ubiquitin amino-terminal subdomain and a cloning site suitable for the insertion of a nucleic acid encoding a polypeptide sequence whereby a fusion protein comprising Nux and the polypeptide sequence can be expressed; and,
instructions indicating that a nucleic acid encoding a defined target protein sequence is to be inserted into the first nucleic acid and members of a library of nucleic acids encoding candidate polypeptides are to be inserted into the second nucleic acid, in order to characterize a polypeptide that binds to the target protein.

83. The kit of claim 82, wherein the active reporter moiety is a negative selectable marker selected from the group consisting of: URA3, Tk, codA, HygTk, Tkneo, TkBSD, PACTk, HygCoda, Codaneo, CodaBSD, PACCoda, GPT2, and HPRT.

84. The kit of claim 82, wherein the active reporter moiety is a negative selectable marker selected from the group consisting of: TRP1, CAN 1, CYH2.

85. The kit of claim 82 wherein the active reporter moiety is selected from the group consisting of: a transcription factor and a fluorescent marker.

86. The kit of claim 82 wherein Nux contains at least one point mutation at amino acid 3 or amino acid 13 of a ubiquitin.

87. The kit of claim 82, wherein the expression of first and second nucleic acids are carried out in a cell.

88. The kit of claim 87, wherein the cell is a eukaryotic cell.

89. The kit of claim 87, wherein the cell is a mammalian cell.

90. The kit of claim 87, wherein the cell is a fungal cell.

91. The kit of claim 87, wherein the cell is a plant cell.

92. The kit of claim 87, wherein the cell is an insect cell.

93. The kit of claim 87, wherein the cell is selected from the group consisting of: a human cell, a mouse cell, a rat cell, a hamster cell, a zebrafish cell, a Drosophila cell, a nematode cell, an S. pombe cell and an S. cerevisiae cell.

94. The kit of claim 87, wherein the cell is selected from the group consisting of: an A. thaliana cell and an N. tabacum cell.

95. The kit of claim 82 wherein said instructions indicate that the library may comprise 10 to 500 members, wherein candidate polypeptides encoded by multiple members of said library are not known to bind said defined target protein.

96. The kit of claim 82 wherein said instructions indicate that the library may comprise 500 to 10,000 members, wherein candidate polypeptides encoded by multiple members of said library are not known to bind said defined target protein.

97. The kit of claim 82 wherein said instructions indicate that the library may comprise greater than 10,000 members, wherein candidate polypeptides encoded by multiple members of said library are not known to bind said defined target protein.

98. A kit for characterizing the sequence of a polypeptide that binds a target protein, which comprises:
a first nucleic acid encoding a target fusion protein comprising a cloning site suitable for the insertion of a nucleic acid encoding a target protein sequence, segments Cub-X, and RM, in an order wherein Cub-X is closer to the N-terminus of the target fusion protein than RM, wherein Cub is the carboxy-terminal subdomain of a wild-type ubiquitin, X is an amino acid selected from the group consisting of arg, lys, phe, leu, trp, his, asp, asn, tyr, ile, glu, cys and gln, and RM is an active reporter moiety, which activity allows for selection, whereby a fusion protein comprising the target protein sequence, Cub-X and RM can be expressed;
a library of second nucleic acids each comprising an Nux segment encoding the amino-terminal subdomain of a wild-type ubiquitin or a reduced-associating mutant ubiquitin amino-terminal subdomain and a nucleic acid encoding a polypeptide sequence, whereby a library of fusion proteins comprising Nux and the polypeptide sequences can be expressed.

99. The kit of claim 98, further comprising instructions indicating that a nucleic acid encoding a defined target protein sequence is to be inserted into the first nucleic acid, in order to characterize a polypeptide that binds to the target protein.

100. The kit of claim 98, wherein the active reporter moiety is a negative selectable marker selected from the group consisting of: URA3, Tk, codA, HygTk, Tkneo, TkBSD, PACTk, HygCoda, Codaneo, CodaBSD, PACCoda, GPT2, and HPRT.

101. The kit of claim 98, wherein the active reporter moiety is a negative selectable marker selected from the group consisting of: TRP1, CAN1, CYH2.

102. The kit of claim 98, wherein the active reporter moiety is selected from the group consisting of: a transcription factor and a fluorescent marker.

103. The kit of claim 98, wherein Nux contains at least one point mutation at amino acid 3 or amino acid 13 of a ubiquitin.

104. The kit of claim 98, wherein the expression of first and second nucleic acids are carried out in a cell.

105. The kit of claim 104, wherein the cell is a eukaryotic cell.

106. The kit of claim 104, wherein the cell is a mammalian cell.

107. The kit of claim 104, wherein the cell is a fungal cell.

108. The kit of claim 104, wherein the cell is a plant cell.

109. The kit of claim 104, wherein the cell is an insect cell.

110. The kit of claim 104, wherein the cell is selected from the group consisting of: a human cell, a mouse cell, a rat cell, a hamster cell, a zebrafish cell, a Drosophila cell, a nematode cell, an S. pombe cell and an S. cerevisiae cell.

111. The kit of claim 104, wherein the cell is selected from the group consisting of: an A. thaliana cell and an N. tabacum cell.

112. The kit of claim 98 wherein said library comprises 10 to 500 members, wherein candidate polypeptides encoded by multiple members of said library are not known to bind said defined target protein.

113. The kit of claim 98 wherein said library comprises 500 to 10,000 members, wherein candidate polypeptides encoded by multiple members of said library are not known to bind said defined target protein.

114. The kit of claim 98 wherein said library comprises greater than 10,000 members, wherein candidate polypeptides encoded by multiple members of said library are not known to bind said defined target protein.
